(19) United States
(12) Patent Application Publication
(10) Pub. No.: US 2025/0248300 A1
(43) Pub. Date: Jul. 31, 2025

(54) ORGANIC LIGHT-EMITTING DEVICE

(71) Applicant: CANON KABUSHIKI KAISHA, Tokyo (JP)

(72) Inventors: YOSUKE NISHIDE, Kanagawa (JP); MASAHITO MIYABE, Kanagawa (JP); HIROKAZU MIYASHITA, Miyagi (JP); NAOKI YAMADA, Tokyo (JP)

(21) Appl. No.: 19/085,930

(22) Filed: Mar. 20, 2025

Related U.S. Application Data
(63) Continuation of application No. PCT/JP2023/034198, filed on Sep. 21, 2023.

(30) Foreign Application Priority Data
Sep. 22, 2022 (JP) 2022-150926
Sep. 15, 2023 (JP) 2023-150097

(51) Int. Cl. H10K 85/60; B41J 2/45; C07C 13/62; C07C 13/66; C07C 211/57; C07D 209/86; C07D 307/91; C07D 319/24; C07D 327/08; C07D 333/76; C07D 345/00; C07D 487/06; C07D 493/04; C07D 495/04; C07D 498/06; C07D 498/22; G03G 15/04; H10K 50/12; H10K 101/40

(52) U.S. Cl. CPC H10K 85/626; B41J 2/45; C07C 13/62; C07C 13/66; C07C 211/57; C07D 209/86; C07D 307/91; C07D 319/24; C07D 327/08; C07D 333/76; C07D 345/00; C07D 487/06; C07D 493/04; C07D 495/04; C07D 498/06; C07D 498/22; G03G 15/04054; H10K 85/622; H10K 85/624; H10K 85/657; H10K 85/6572; C07B 2200/05; H10K 50/12; H10K 2101/40

(57) ABSTRACT

An organic light-emitting device that is highly efficient and has excellent driving durability characteristics. The present disclosure provides an organic light-emitting device including a substrate, a first electrode, a light-emitting layer, and a second electrode. The light-emitting layer contains a first organic compound, a second organic compound, and a third organic compound. A freely rotatable single bond in each of the first to third organic compounds is a carbon-carbon bond. At least one carbon of the freely rotatable carbon-carbon bond in the second organic compound is an sp² carbon atom. Letting the HOMO energy level of the first organic compound be HOMO1, letting the LUMO energy level of the second organic compound be LUMO2, and letting the HOMO and LUMO energy levels of the third organic compound be HOMO3 and LUMO3, [I] and [II] are satisfied:

|LUMO2|>|LUMO3| [I]

|HOMO3|>|HOMO1| [II].

### CROSS-REFERENCE TO RELATED APPLICATIONS

This application is a Continuation of International Patent Application No. PCT/JP2023/034198, filed Sep. 21, 2023, which claims the benefit of Japanese Patent Application No. 2022-150926, filed Sep. 22, 2022, and Japanese Patent Application No. 2023-150097, filed Sep. 15, 2023, all of which are hereby incorporated by reference herein in their entirety.

### TECHNICAL FIELD

The present invention relates to organic light-emitting devices, various devices and apparatuses including the organic light-emitting devices, and organic compounds for use in the organic light-emitting devices.

### BACKGROUND ART

Organic light-emitting devices (hereinafter, also referred to as “organic electroluminescent devices” or “organic EL devices”) are devices each including a pair of electrodes and an organic compound layer disposed between these electrodes. The injection of electrons and holes from the pair of electrodes generates excitons of the light-emitting organic compound in the organic compound layer, and when the excitons return to the ground state, the organic light-emitting device emits light.

Recent advances in organic light-emitting devices have been remarkable, enabling features, such as low driving voltage, various emission wavelengths, fast response time, and achieving thinner and lighter devices.

Organic light-emitting devices can emit light of various colors. In particular, improving the durability performance of blue-light-emitting devices, which have the highest energy in the visible region, is a challenge.

One example of a method for improving the durability performance of a device is to improve the configuration of a light-emitting layer. Patent Literature 1 reports an example of an organic green-light-emitting device having a ternary light-emitting layer containing a pyrene-based host, a fluoranthene dopant having a tert-butyl group, and a third organic compound. Patent Literature 2 reports an example of an organic blue-light-emitting device having a ternary light-emitting layer containing a carbazole-based host, a dopant having a diarylamino group, and a third organic compound.

To provide a display with even higher reliability, an organic light-emitting device having a longer life is required. Improving durability characteristics, especially in a high-energy organic blue-light-emitting device, is a challenge.

Patent Literature 1 discloses an example of a light-emitting device in which the HOMO and LUMO levels in a light-emitting layer are adjusted to improve durability characteristics through the use of three types of organic compounds. However, a dopant containing an sp³ carbon-sp³ carbon bond having low bond energy is used, and thus a further improvement in durability performance is desired.

Patent Literature 2 discloses an example of a blue-light-emitting device in which the HOMO and LUMO levels in a light-emitting layer are adjusted to improve durability characteristics through the use of three types of organic compounds. However, a dopant containing a carbon-nitrogen single bond having low bond energy is used, and thus a further improvement in durability performance is desired.

### CITATION LIST

### Patent Literature

PTL 1 Japanese Patent Laid-Open No. 2011-71194
PTL 2 Japanese Patent Laid-Open No. 2010-245061

### SUMMARY OF INVENTION

The present invention has been accomplished in light of the above problems, and it is an object of the present invention to provide an organic light-emitting device having excellent durability characteristics.

The present invention provides an organic light-emitting device including a first electrode, a light-emitting layer, and a second electrode,
in which the light-emitting layer contains a first organic compound, a second organic compound, and a third organic compound,
a freely rotatable single bond in each of the first organic compound, the second organic compound, and the third organic compound is a carbon-carbon bond,
at least one carbon atom of a freely rotatable carbon-carbon bond contained in the second organic compound is an sp² carbon atom, and
letting the HOMO energy level of the first organic compound be HOMO1, letting the LUMO energy level of the second organic compound be LUMO2, and letting the HOMO and LUMO energy levels of the third organic compound be HOMO3 and LUMO3, [I] and [II] are satisfied:

|LUMO2|>|LUMO31 [I]

|HOMO3|>|HOMO1| [II].

Further features of the present invention will become apparent from the following description of exemplary embodiments with reference to the attached drawings.

### BRIEF DESCRIPTION OF DRAWINGS

FIG. 1A is a schematic cross-sectional view illustrating an example of a pixel of a display apparatus according to an embodiment of the present invention.

FIG. 1B is a schematic cross-sectional view of an example of a display apparatus including organic light-emitting devices according to an embodiment of the present invention.

FIG. 2 is a schematic view illustrating an example of a display apparatus according to an embodiment of the present invention.

FIG. 3A is a schematic view illustrating an example of an image pickup apparatus according to an embodiment of the present invention.

FIG. 3B is a schematic view illustrating an example of an electronic apparatus according to an embodiment of the present invention.

FIG. 4A is a schematic view illustrating an example of a display apparatus according to an embodiment of the present invention.

FIG. 4B is a schematic view illustrating an example of a foldable display apparatus.

FIG. 5A is a schematic view illustrating an example of a lighting apparatus according to an embodiment of the present invention.

FIG. 5B is a schematic view illustrating an example of an automobile including an automotive lighting unit according to an embodiment of the present invention.

FIG. 6A is a schematic view illustrating an example of a wearable device according to an embodiment of the present invention.

FIG. 6B is a schematic view illustrating an example of a wearable device according to an embodiment of the present invention, the wearable device including an image pickup apparatus.

FIG. 7A is a schematic view of an example of an image-forming apparatus according to an embodiment of the present invention.

FIG. 7B is a schematic view illustrating a configuration of an exposure light source including multiple light-emitting portions arranged on a long substrate.

FIG. 7C is a schematic view illustrating a configuration of an exposure light source including multiple light-emitting portions arranged on a long substrate.

### DESCRIPTION OF EMBODIMENTS

The present invention is an invention for providing a long-life organic light-emitting device. To achieve a longer life for an organic light-emitting device, it is necessary to inhibit the deterioration of an organic material in the device, particularly an organic material used in a light-emitting layer. Typically, in order to extend the life of an organic light-emitting device, it is effective to expand the recombination zone to inhibit deterioration due to the concentration of exciton generation. When exciton generation is concentrated, an energy transfer from a molecule in an excited state to another molecule in an excited state causes a transition to a higher-energy state. In a high-energy state, when a situation occurs in which the bond energy of a single bond site in the molecular structure is exceeded, the bond is cleaved to generate decomposition products, causing a decrease in luminance. To avoid this, it is effective to disperse exciton generation.

There are several methods to disperse the exciton generation zone. In the present invention, we considered that the addition of two types of organic compounds, i.e., a hole-trapping first organic compound and an electron-trapping second organic compound, to the host compound would be an effective method to ensure good charge injection into a light-emitting layer and to disperse the exciton generation zone. In other words, the following relationships [I] and [II] are satisfied. A third organic compound is a host.

|LUMO2|>|LUMO3| [I]

|HOMO3|>|HOMO1| [II]
HOMO1: The HOMO energy level of the first organic compound.
HOMO3: The HOMO energy level of the third organic compound.
LUMO2: The LUMO energy level of the second organic compound.
LUMO3: The LUMO energy level of the third organic compound.

HOMO refers to the highest occupied molecular orbital. LUMO refers to the lowest unoccupied molecular orbital. A HOMO energy level is also referred to as “HOMO” or a “HOMO level”. A LUMO energy level is also referred to as “LUMO” or a “LUMO level”.

The features of the configuration of the light-emitting layer in the present invention will be described below. In this specification, specific examples of substituents are described below, unless otherwise specified.

An alkyl group may be an alkyl group having 1 or more and 20 or less carbon atoms. Examples thereof include, but are not limited to, a methyl group, an ethyl group, a n-propyl group, an isopropyl group, a n-butyl group, a tert-butyl group, a sec-butyl group, an octyl group, a cyclohexyl group, a 1-adamantyl group, and a 2-adamantyl group.

An alkoxy group may be an alkoxy group having 1 or more and 10 or less carbon atoms. Examples thereof include, but are not limited to, a methoxy group, an ethoxy group, a propoxy group, a 2-ethyloctyloxy group, and a benzyloxy group.

Examples of a silyl group include, but are not limited to, a trimethylsilyl group and a triphenylsilyl group.

An aryl group may be an aryl group having 6 or more and 20 or less carbon atoms. Examples thereof include, but are not limited to, a phenyl group, a naphthyl group, an indenyl group, a biphenyl group, a terphenyl group, a fluorenyl group, a phenanthryl group, a fluoranthenyl group, and a triphenylenyl group.

A heteroaryl group may be a heteroaryl group having 3 or more and 20 or less carbon atoms. Examples thereof include, but are not limited to, a pyridyl group, a pyrimidinyl group, a pyrazinyl group, a triazolyl group, an oxazolyl group, an oxadiazolyl group, a thiazolyl group, a thiadiazolyl group, a carbazolyl group, an acridinyl group, a phenanthrolinyl group, a dibenzofuranyl group, and a dibenzothiophenyl group.

Examples of an amino group include, but are not limited to, an N-methylamino group, an N-ethylamino group, an N,N-dimethylamino group, an N,N-diethylamino group, an N-methyl-N-ethylamino group, an N-benzylamino group, an N-methyl-N-benzylamino group, an N,N-dibenzylamino group, an anilino group, an N,N-diphenylamino group, an N,N-dinaphthylamino group, an N,N-difluorenylamino group, an N-phenyl-N-tolylamino group, an N,N-ditolylamino group, an N-methyl-N-phenylamino group, an N,N-dianisolylamino group, an N-mesityl-N-phenylamino group, an N,N-dimesitylamino group, an N-phenyl-N-(4-tert-butylphenyl)amino group, an N-phenyl-N-(4-trifluoromethylphenyl)amino group, an N-piperidyl group, and a carbazolyl group.

Examples of the aryloxy group and the heteroaryloxy group include, but are not limited to, a phenoxy group and a thienyloxy group.

Examples of substituents that may be further contained in the alkyl group, the alkoxy group, the silyl group, the aryl group, the heteroaryl group, the amino group, the aryloxy group, and the heteroaryloxy group include, but are not limited to, a deuterium atom; halogen atoms, such as fluorine, chlorine, bromine, and iodine; alkyl groups, such as a methyl group, an ethyl group, a n-propyl group, an isopropyl group, a n-butyl group, and a tert-butyl group; alkoxy groups, such as a methoxy group, an ethoxy group, and a propoxy group; amino groups, such as a dimethylamino group, a diethylamino group, a dibenzylamino group, a diphenyl amino group, and a ditolylamino group; aryloxy groups, such as a phenoxy group; aromatic hydrocarbon groups, such as a phenyl group and a biphenyl group; heteroaryl groups, such as a pyridyl group and a pyrrolyl group; and a cyano group, a hydroxy group, and a thiol group.

[1] A freely rotatable single bond in a compound contained in the light-emitting layer is a carbon-carbon bond.

One of the features of the organic light-emitting device of the present invention is that holes in the light-emitting layer are mainly trapped on the first organic compound, and electrons in the light-emitting layer are mainly trapped on the second organic compound. This enables charges to be confined in the light-emitting layer, and recombination zones are dispersed, thereby improving durability.

In other words, this indicates that the first organic compound and the second organic compound are subjected to a load in a radical state or an excited state. Therefore, each of the first organic compound and the second organic compound is required to have a skeleton that is resistant to decomposition even in a high-energy excited state. Furthermore, the third organic compound, which is a host, also desirably has a skeleton that is similarly resistant to decomposition because charge recombination and excitation occur. These are features of the present invention. Specifically, it is important that all of the freely rotatable single bonds in each skeleton that is resistant to decomposition be carbon-carbon bonds. Furthermore, at least one carbon atom in each freely rotatable single bond is preferably an sp² carbon atom. Moreover, all of the freely rotatable single bonds are preferably sp² carbon-sp² carbon bonds. Note that sp² and sp³ indicate an sp² hybrid orbital and an sp³ hybrid orbital, respectively.

Table 1 presents examples of bond energies of freely rotatable single bonds. As presented in Table 1, when a carbon-nitrogen bond or the like is provided as a bond between fused polycyclic skeletons, the bond breaks easily in a higher-energy excited state that occurs due to a certain concentration of exciton generation. In the present invention, therefore, the freely rotatable single bond in each of the first organic compound, the second organic compound, and the third organic compound is a carbon-carbon bond.

TABLE 1

| Freely rotatable single | | Bond energy |
|---|---|---|
| bond | Representative molecular structure | [eV] |
| sp² Carbon-nitrogen | 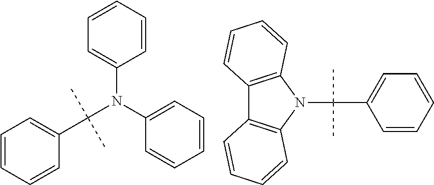 | 3.8 |
| sp³ Carbon-sp³ carbon | 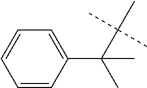 | 4.3 |
| sp² Carbon-sp³ carbon | 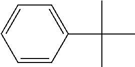 | 4.9 |
| sp² Carbon-sp² carbon | 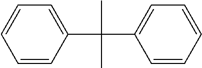 | 5.1 |

Table 2 presents comparisons in terms of durability characteristics between organic light-emitting devices each having light-emitting layer configuration and containing materials described in Patent Literature 2 and an organic light-emitting device having a light-emitting layer configuration and containing materials of the present invention. In Table 2, “Present Invention 1” and “Comparative Example 1” to “Comparative Example 3” correspond to Example 1 and Comparative Examples 1 to 3 in Examples described below. The LUMO level and the HOMO level are expressed in units of [eV]. A method for producing an organic light-emitting device, layer configurations, evaluations, and methods for measuring the HOMO level and the LUMO level are described in the Examples below.

TABLE 2

| | | | | | | | | Device |
|---|---|---|---|---|---|---|---|---|
| | | Third organic | | First organic | | Second organic | | durability |
| | | compound | | compound | | compound | | ratio |
| Present Invention 1 | LUMO HOMO | −3.0 −6.0 | 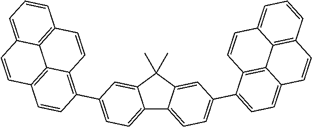 [SMILES: CC1(C)c2cc(-c3ccc4ccc5cccc6ccc3c4c56)ccc2-c2ccc(-c3ccc4ccc5cccc6ccc3c4c56)cc21] | −2.8 −5.8 | 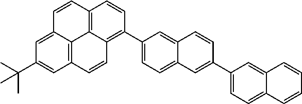 [SMILES: CC(C)(C)c1cc2ccc3ccc(-c4ccc5cc(-c6ccc7ccccc7c6)ccc5c4)c4ccc(c1)c2c34] | −3.2 −6.0 | 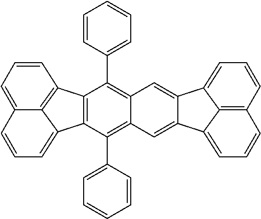 [SMILES: c1ccc(-c2c3cc4c(cc3c(-c3ccccc3)c3c5cccc6cccc(c23)c65)c2cccc3cccc4c32)cc1] | 1.3 |
| Comparative Example 1 | LUMO HOMO | −3.0 −6.0 | 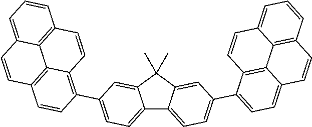 [SMILES: CC1(C)c2cc(-c3ccc4ccc5cccc6ccc3c4c56)ccc2-c2ccc(-c3ccc4ccc5cccc6ccc3c4c56)cc21] | −2.6 −5.6 | 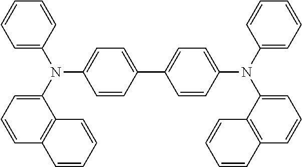 [SMILES: c1ccc(N(c2ccc(-c3ccc(N(c4ccccc4)c4cccc5ccccc45)cc3)cc2)c2cccc3ccccc23)cc1] | −3.2 −6.0 | 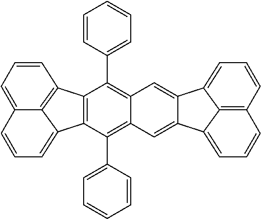 [SMILES: c1ccc(-c2c3cc4c(cc3c(-c3ccccc3)c3c5cccc6cccc(c23)c65)c2cccc3cccc4c32)cc1] | 1.0 |
| Comparative Example 2 | LUMO HOMO | −3.0 −6.0 | 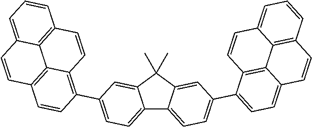 [SMILES: CC1(C)c2cc(-c3ccc4ccc5cccc6ccc3c4c56)ccc2-c2ccc(-c3ccc4ccc5cccc6ccc3c4c56)cc21] | −2.6 −5.5 | 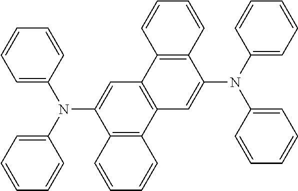 [SMILES: c1ccc(N(c2ccccc2)c2cc3c4ccccc4c(N(c4ccccc4)c4ccccc4)cc3c3ccccc23)cc1] | −3.1 −6.0 | 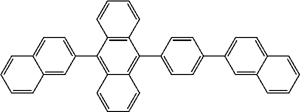 [SMILES: c1ccc2cc(-c3ccc(-c4c5ccccc5c(-c5ccc6ccccc6c5)c5ccccc45)cc3)ccc2c1] | 0.7 |
| Comparative Example 3 | LUMO HOMO | −2.7 −6.2 | 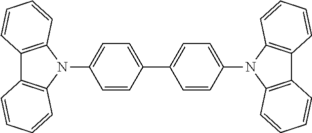 [SMILES: c1ccc2c(c1)c1ccccc1n2-c1ccc(-c2ccc(-n3c4ccccc4c4ccccc43)cc2)cc1] | −2.9 −5.5 | 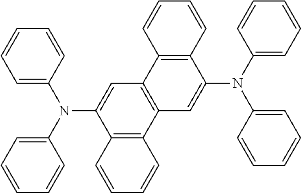 [SMILES: c1ccc(N(c2ccccc2)c2cc3c4ccccc4c(N(c4ccccc4)c4ccccc4)cc3c3ccccc23)cc1] | −3.1 −6.0 | 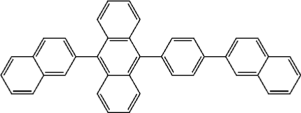 [SMILES: c1ccc2cc(-c3ccc(-c4c5ccccc5c(-c5ccc6ccccc6c5)c5ccccc45)cc3)ccc2c1] | 0.5 |

As presented in Table 2, in each of Comparative Examples 1 to 3, in which the freely rotatable single bonds in the materials of the light-emitting layer included C—N bonds, the durability ratio was 1.0 or less. In contrast, in Present Invention 1, in which all freely rotatable single bonds in the materials of the light-emitting layer were carbon-carbon bonds, the durability ratio was 1.3. Thus, all of the freely rotatable single bonds in the materials contained in the light-emitting layer are preferably carbon-carbon bonds having high bond energies.

[2] At least one carbon atom of each freely rotatable carbon-carbon bond of the second organic compound is an sp² carbon atom.

In the present invention, the second organic compound is an electron-trapping compound with a deep LUMO level (far from the vacuum level). The first organic compound is a hole-trapping compound with a shallow HOMO level. In all of the first to third organic compounds, all of the freely rotatable single bonds are composed of carbon-carbon bonds. Thus, the first organic compound and the second organic compound both tend to have deep HOMO-LUMO. In other words, the difference between the LUMO levels of the third organic compound and the second organic compound (electron-trapping performance) is larger than the difference between the HOMO levels of the third organic compound and the first organic compound (hole-trapping performance). Among the organic compounds contained in the light-emitting layer, the second organic compound has the highest probability of trapping charges and being excited, and is easily decomposed by charges and excitons. Therefore, among the first to third organic compounds, in particular, the second organic compound preferably has high bond stability.

As presented in Table 1, the stability of bond energy increases in the order of the sp² carbon-nitrogen bond, the sp³ carbon-sp³ carbon bond, the sp³ carbon-sp² carbon bond, and the sp² carbon-sp² carbon bond. These results indicate that, among the carbon-carbon bonds, the sp³ carbon-sp³ carbon bond has low bond energy and is prone to decomposition or reaction in an excited state, easily causing significant luminance degradation during operation of the organic light-emitting device. That is, the durability characteristics of the organic light-emitting device deteriorate easily. The sp² carbon-sp³ carbon bond or the sp² carbon-sp² carbon bond has a higher bond energy than the sp³ carbon-sp³ carbon bond, and decomposition or reaction in an excited state is inhibited, so that the durability characteristics of the organic light-emitting device are improved.

Table 3 presents comparisons between an organic light-emitting device including a light-emitting layer composed of materials described in Patent Literature 1 and organic light-emitting devices including light-emitting layers composed of materials of the present invention. In Table 3, “Present Invention 2” to “Present Invention 4” and “Comparative Example 4” correspond to Examples 2 to 4 and Comparative Example 4 in Examples described below. The LUMO level and the HOMO level are expressed in units of [eV]. A method for producing an organic light-emitting device, layer configurations, evaluations, and methods for measuring the HOMO level and the LUMO level are described in the Examples below.

TABLE 3

| | | | | | | | | Device |
|---|---|---|---|---|---|---|---|---|
| | | Third organic | | First organic | | Second organic | | durability |
| | | compound | | compound | | compound | | ratio |
| Present Invention 2 | LUMO HOMO | −3.1 −6.1 | 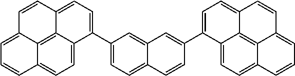 [SMILES: c1cc2ccc3ccc(-c4ccc5ccc(-c6ccc7ccc8cccc9ccc6c7c89)cc5c4)c4ccc(c1)c2c34] | −3.0 −5.9 | 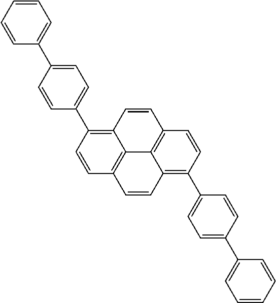 [SMILES: c1ccc(-c2ccc(-c3ccc4ccc5c(-c6ccc(-c7ccccc7)cc6)ccc6ccc3c4c65)cc2)cc1] | −3.5 −6.1 | 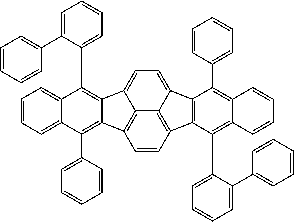 [SMILES: c1ccc(-c2ccccc2-c2c3c(c(-c4ccccc4)c4ccccc24)-c2ccc4c5c(ccc-3c25)-c2c-4c(-c3ccccc3-c3ccccc3)c3ccccc3c2-c2ccccc2)cc1] | 2.7 |
| Present Invention 3 | LUMO HOMO | −3.0 −6.0 | 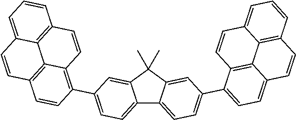 [SMILES: CC1(C)c2cc(-c3ccc4ccc5cccc6ccc3c4c56)ccc2-c2ccc(-c3ccc4ccc5cccc6ccc3c4c56)cc21] | −3.1 −5.9 | 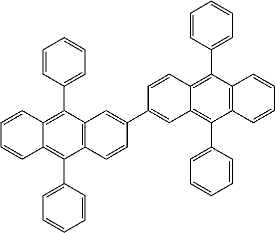 [SMILES: c1ccc(-c2c3ccccc3c(-c3ccccc3)c3cc(-c4ccc5c(-c6ccccc6)c6ccccc6c(-c6ccccc6)c5c4)ccc23)cc1] | −3.5 −6.1 | 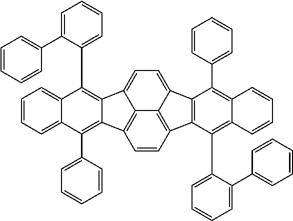 [SMILES: c1ccc(-c2ccccc2-c2c3c(c(-c4ccccc4)c4ccccc24)-c2ccc4c5c(ccc-3c25)-c2c-4c(-c3ccccc3-c3ccccc3)c3ccccc3c2-c2ccccc2)cc1] | 1.7 |
| Present Invention 4 | LUMO HOMO | −3.0 −6.0 | 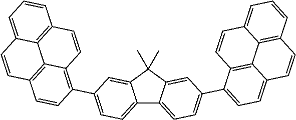 [SMILES: CC1(C)c2cc(-c3ccc4ccc5cccc6ccc3c4c56)ccc2-c2ccc(-c3ccc4ccc5cccc6ccc3c4c56)cc21] | −3.1 −5.9 | 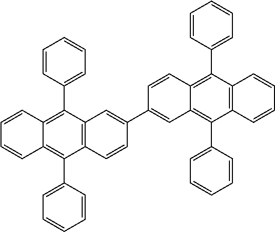 [SMILES: c1ccc(-c2c3ccccc3c(-c3ccccc3)c3cc(-c4ccc5c(-c6ccccc6)c6ccccc6c(-c6ccccc6)c5c4)ccc23)cc1] | −3.6 −6.0 | 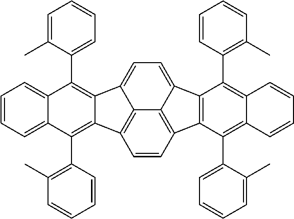 [SMILES: Cc1ccccc1-c1c2c(c(-c3ccccc3C)c3ccccc13)-c1ccc3c4c(ccc-2c14)-c1c-3c(-c2ccccc2C)c2ccccc2c1-c1ccccc1C] | 1.5 |
| Comparative Example 4 | LUMO HOMO | −3.0 −6.0 | 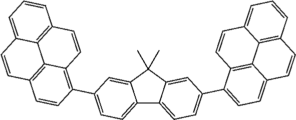 [SMILES: CC1(C)c2cc(-c3ccc4ccc5cccc6ccc3c4c56)ccc2-c2ccc(-c3ccc4ccc5cccc6ccc3c4c56)cc21] | −3.1 −5.9 | 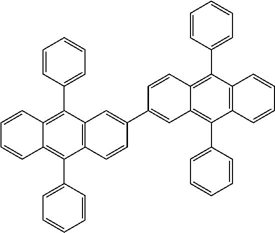 [SMILES: c1ccc(-c2c3ccccc3c(-c3ccccc3)c3cc(-c4ccc5c(-c6ccccc6)c6ccccc6c(-c6ccccc6)c5c4)ccc23)cc1] | −3.6 −6.0 | 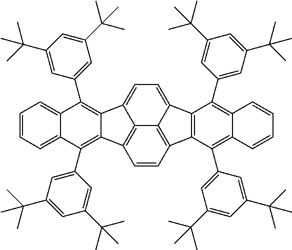 [SMILES: CC(C)(C)c1cc(-c2c3c(c(-c4cc(C(C)(C)C)cc(C(C)(C)C)c4)c4ccccc24)-c2ccc4c5c(ccc-3c25)-c2c-4c(-c3cc(C(C)(C)C)cc(C(C)(C)C)c3)c3ccccc3c2-c2cc(C(C)(C)C)cc(C(C)(C)C)c2)cc(C(C)(C)C)c1] | 1.0 |

Table 3 indicates that better durability characteristics are exhibited in Present Invention 4, in which the second organic compound has no sp³ carbon-sp³ carbon bond, than in Comparative Example 4, in which the second organic compound has sp³ carbon-sp³ carbon bonds as freely rotatable single bonds. In addition, even better durability characteristics are exhibited in Present Invention 3, in which the second organic compound has the sp² carbon-sp² carbon bonds only, than in Present Invention 4, in which the second organic compound has the sp² carbon-sp³ carbon bonds. Furthermore, still even better durability characteristics are exhibited in Present Invention 2, in which the freely rotatable single bonds are sp² carbon-sp² carbon bonds in all of the first to third organic compounds. Therefore, the second organic compound according to the present invention is an organic compound composed only of an sp³ carbon-sp² carbon bond and an sp² carbon-sp² carbon bond, i.e., an organic compound having a carbon-carbon bond in which at least one carbon is sp² carbon, thus resulting in improved durability characteristics of the organic light-emitting device.

More preferably, the present invention further has the following characteristics.

[3] The second organic compound has a fluoranthene skeleton.

As described above, the second organic compound is characterized by having a deep LUMO level (far from the vacuum level) relative to the host (the third organic compound) and having an electron-trapping property. The second organic compound preferably has a fluoranthene skeleton as the skeleton having a deep LUMO level. This is because the fluoranthene skeleton has an electron-deficient five-membered ring and is composed only of stable hydrocarbons. Furthermore, the fluoranthene skeleton has two or more five-membered rings and thus has a deeper LUMO level and improved electron-trapping property, which is preferred.

Typically, the material of an electron-blocking layer has a freely rotatable sp² carbon-nitrogen bond and is electron-rich in a neutral state; hence, the material is easily decomposed by receiving electrons (reduction) or in an excited state. Thus, in a device configuration including an electron-blocking layer or a hole-blocking layer, the durability characteristics of the device are improved by localizing a recombination zone closer to the hole blocking layer/light-emitting layer interface than to the electron-blocking layer/light-emitting layer interface. For this reason, preferably, the second organic compound has a deep LUMO relative to the host compound and has an electron-trapping property. It is more preferable to use a first organic compound in which a freely rotatable single bond consists of a carbon-carbon bond in the electron-blocking layer, because decomposition is inhibited from the viewpoint of the stability of the molecular structure.

[4] The first organic compound is stable against oxidation.

As mentioned above, the first organic compound has a high probability of being present in a radical cation state. Thus, preferably, the first organic compound is particularly stable in a radical cation state. Table 4 presents the results of evaluating the oxidation stability of two organic compounds by performing 10 consecutive sweep evaluations of one-electron oxidation using cyclic voltammetry (CV) measurement. The CV measurement was performed in a 0.1 M solution of tetrabutylammonium perchlorate in dichloromethane using an Ag/Ag⁺ reference electrode, a Pt counter electrode, and a glassy carbon working electrode. The sweep rate was 0.1 V/s. As a measurement device, a Model 660C electrochemical analyzer manufactured by ALS was used. The device durability ratio was evaluated using a device configuration similar to that of the Present Invention 2 (Example 2) in Table 3.

TABLE 4

| | | CV first oxidation | |
|---|---|---|---|
| | | wave | Device |
| | | (10 repeated | durability |
| | Molecular structure | sweeps) | ratio |
| Organic compound 1 | 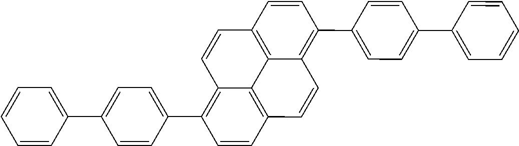 [SMILES: c1ccc(-c2ccc(-c3ccc4ccc5c(-c6ccc(-c7ccccc7)cc6)ccc6ccc3c4c65)cc2)cc1] | reversible | 1.4 |
| Organic compound 2 | 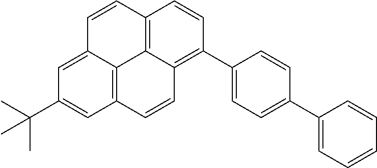 [SMILES: CC(C)(C)c1cc2ccc3ccc(-c4ccc(-c5ccccc5)cc4)c4ccc(c1)c2c34] | irreversible | 1.0 |

As presented in Table 4, it was found that organic compound 1 from which a reversible oxidation wave was obtained had a device durability ratio 1.4 times higher than that of organic compound 2 from which an irreversible oxidation wave was obtained. This indicates that organic compound 1, which has a molecular structure with high oxidation stability, can be stably present without decomposition or reaction even when subjected to one-electron oxidation.

[5] The freely rotatable single bonds in the first to third organic compounds are all sp² carbon-sp² carbon bonds.

As described in [2], the freely rotatable single bond in the second organic compound preferably has a higher bond energy, and is preferably an sp² carbon-sp² carbon bond having the highest bond energy. However, charge recombination and excitation also occur in the first organic compound and the third organic compound, which is a host, to a greater or lesser extent. Thus, more preferably, the freely rotatable single bonds in the first organic compound and the third organic compound contained in the light-emitting layer are also composed of only sp² carbon-sp² carbon bonds.

[6] When the freely rotatable single bond of the first organic compound is an sp² carbon-sp² carbon bond, the first organic compound is preferably an organic compound represented by general formula [1]. More preferably, the first organic compound is an organic compound represented by the following general formula [1] and composed only of a hydrocarbon.

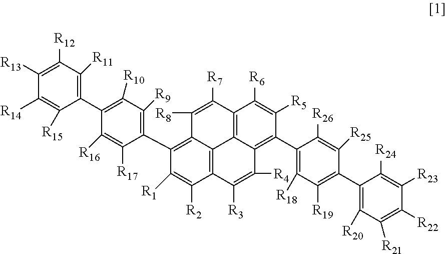

In general formula [1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group. R₁₀ and R₁₁, R₁₅ and R₁₆, R₁₉ and R₂₀, and R₂₄ and R₂₅ may form respective rings, each ring containing a carbon atom, an oxygen atom, a sulfur atom, a selenium atom, or a tellurium atom serving as a spacer. R₁₁ to R₁₅ and R₂₀ to R₂₄ may each form a ring with an adjacent substituent.

The organic compound represented by general formula [1] has a hole-trapping property and thus has a high excitation probability and high bond energy. In addition, the organic compound has high oxidation stability and thus is preferable as the first organic compound.

In the present invention, the first organic compound is characterized in that it has a shallow HOMO level (close to the vacuum level) relative to the third organic compound serving as a host, and has a hole-trapping property. However, in general, it is very difficult to create a compound in which the freely rotatable single bonds are composed only of sp² carbon-sp² carbon bonds and the HOMO level is shallow. The inventors have intensive studies and have found that the HOMO level is shallower when the molecular structure of general formula [1] is contained. Specifically, when a biphenyl unit is added to a pyrene skeleton, which is also useful as the basic skeleton of the third organic compound or the second organic compound, the compound is stacked more effectively in a thin film, increasing the electron density of the molecule and resulting in a shallower HOMO level. Furthermore, when two or more biphenyl units are added, the HOMO level is even shallower.

Table 5 presents the calculated values and measured values of the HOMO levels and their differences (ΔHOMO) for the presence or absence of biphenyl units and their substitution numbers in the organic compounds related to the first organic compound. The measured values of the HOMO levels are values of the ionization potential measured by preparing a 50-nm films and using “AC-3” manufactured by Riken Keiki Co., Ltd. It was found that the calculated HOMO values of organic compounds 1 to 5 in Table 5 were all equivalent, but the measured HOMO values were shallower as the number of biphenyl units increased. In other words, the effect of making the HOMO shallower by the addition of the biphenyl units was not predicted by the calculations and was found by the inventors.

TABLE 5

| | | | | ΔHOMO |
|---|---|---|---|---|
| | | | | Calculated |
| | | Calculated | Measured | value- |
| Organic | | HOMO | HOMO | measured |
| compound | Molecular structure | value [eV] | value [eV] | value [eV] |
| 1 | 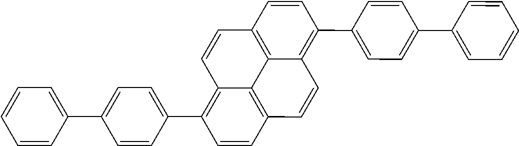 [SMILES: c1ccc(-c2ccc(-c3ccc4ccc5c(-c6ccc(-c7ccccc7)cc6)ccc6ccc3c4c65)cc2)cc1] | −5.13 | −5.89 | 0.76 |
| 2 | 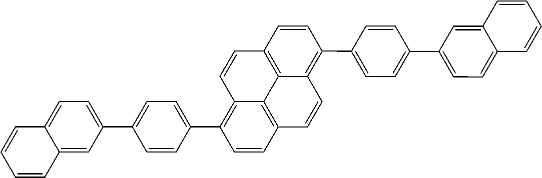 [SMILES: c1ccc2cc(-c3ccc(-c4ccc5ccc6c(-c7ccc(-c8ccc9ccccc9c8)cc7)ccc7ccc4c5c76)cc3)ccc2c1] | −5.14 | −5.92 | 0.78 |
| 3 | 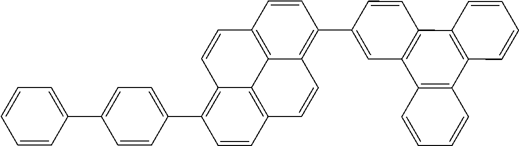 [SMILES: c1ccc(-c2ccc(-c3ccc4ccc5c(-c6ccc7c8ccccc8c8ccccc8c7c6)ccc6ccc3c4c65)cc2)cc1] | −5.14 | −5.97 | 0.83 |
| 4 | 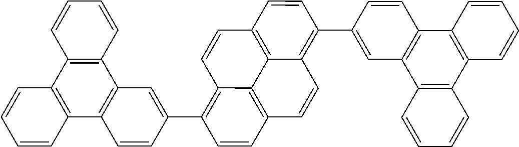 [SMILES: c1ccc2c(c1)c1ccccc1c1cc(-c3ccc4ccc5c(-c6ccc7c8ccccc8c8ccccc8c7c6)ccc6ccc3c4c65)ccc21] | −5.14 | −6.00 | 0.86 |
| 5 | 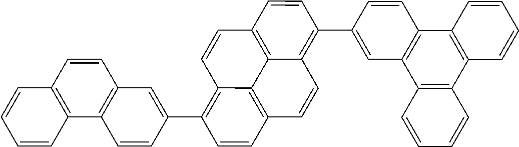 [SMILES: c1ccc2c(c1)ccc1cc(-c3ccc4ccc5c(-c6ccc7c8ccccc8c8ccccc8c7c6)ccc6ccc3c4c65)ccc12] | −5.14 | −6.00 | 0.86 |

When the freely rotatable single bonds in the first organic compound are composed only of sp² carbon-sp² carbon bonds, the use of an organic compound having a shallower HOMO, as represented by general formula [1], improves the hole-trapping performance, further improving the durability characteristics of the organic light-emitting device.

The first organic compound is preferably an organic compound represented by any one of the following general formulae [1-1] to [1-3]. The general formula [1-1] represents a compound in which at least one pair of R₁₀ and R₁₁, R₁₅ and R₁₆, R₁₉ and R₂₀, and R₂₄ and R₂₅ in general formula [1] forms a ring.

The first organic compound is more preferably an organic compound represented by the following general formula [1-3].

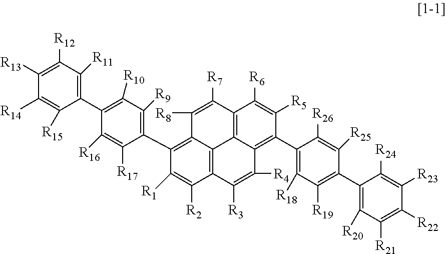

In general formula [1-1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group. At least one pair of R₁₀ and R₁₁, R₁₅ and R₁₆, R₁₉ and R₂₀, and R₂₄ and R₂₅ forms a ring with a carbon atom, an oxygen atom, a sulfur atom, a selenium atom, or a tellurium atom serving as a spacer. R₁₁ to R₁₅ and R₂₀ to R₂₄ may each form a ring with an adjacent substituent.

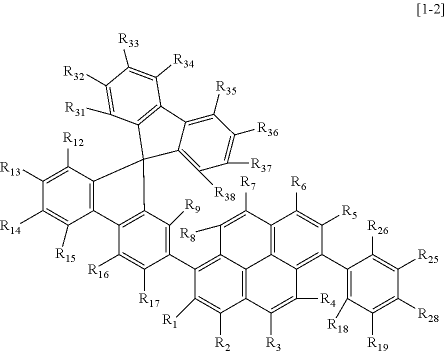

In general formula [1-2], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group. R₁₈ and R₁₉, R₂₅ and R₂₆, and R₂₈ may each form a ring with an adjacent substituent.

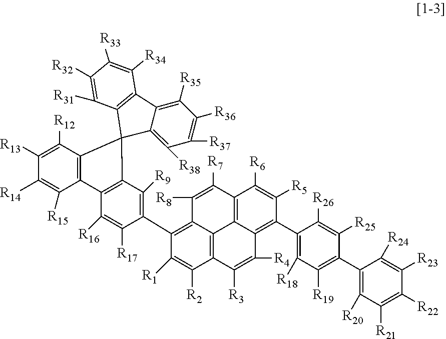

In general formula [1-3], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group. At least one pair of R₁₉ and R₂₀, and R₂₄ and R₂₅ forms a ring with a carbon atom, an oxygen atom, a sulfur atom, a selenium atom, or a tellurium atom serving as a spacer. R₂₀ to R₂₄ may each form a ring with an adjacent substituent.

The organic compound represented by any one of general formulae [1-1] to [1-3] is preferred for the following three reasons.
(i) The high glass transition temperature (Tg) results in high thermal stability and improved driving durability.
(ii) The shallow HOMO results in a strong hole-trapping property and improved driving durability.
(iii) The narrow band gap results in a low injection barrier and a low driving voltage.

Each item will be described below.
(i) The high glass transition temperature (Tg) results in high thermal stability and improved driving durability.

High thermal stability, for example, a high glass transition temperature (Tg), is preferred for use in an organic light-emitting device. This is because the high Tg makes it difficult for grain boundaries, trap levels, and quenchers to be generated due to minute crystallization even during driving of the device, so that a good carrier transport property and high-efficiency light emission characteristics can be maintained. Therefore, an organic light-emitting device having excellent durability and efficiency can be provided.

Table 6 presents the evaluation results of Tg of organic compounds Z-3, Z-4, and Z-18 to 20 described below by differential scanning calorimetry (DSC). It can be said that a higher glass transition temperature indicates higher amorphous nature and better thermal stability. Tg is preferably 120° C. or higher, more preferably 130° C. or higher, and even more preferably 140° C. or higher.

In the DSC measurement, about 2 mg of a sample was sealed in an aluminum pan and then quenched from a high temperature higher than the melting point. This caused the sample to be in an amorphous state. The sample was then heated at a rate of temperature increase of 20° C./min to measure the glass transition temperature. DSC 204 F1 manufactured by NETZSCH was used as a measurement device.

TABLE 6

| Organic | | S1 | HOMO | Tg |
|---|---|---|---|---|
| compound | Molecular structure | [eV] | [eV] | [° C.] |
| Z-4 | 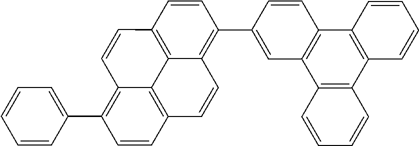 [SMILES: c1ccc(-c2ccc3ccc4c(-c5ccc6c7ccccc7c7ccccc7c6c5)ccc5ccc2c3c54)cc1] | 3.0 | −6.0 | 124 |
| Z-3 | 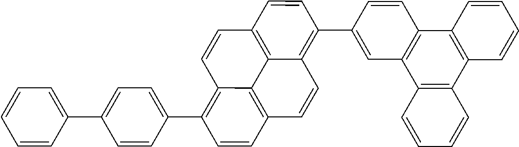 [SMILES: c1ccc(-c2ccc(-c3ccc4ccc5c(-c6ccc7c8ccccc8c8ccccc8c7c6)ccc6ccc3c4c65)cc2)cc1] | 2.8 | −6.0 | 148 |
| Z-18 | 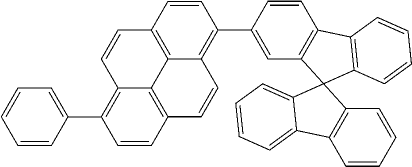 [SMILES: c1ccc(-c2ccc3ccc4c(-c5ccc6c(c5)C5(c7ccccc7-c7ccccc75)c5ccccc5-6)ccc5ccc2c3c54)cc1] | 3.0 | −5.9 | 151 |
| Z-19 | 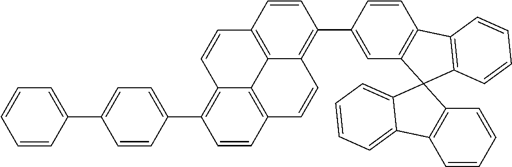 [SMILES: c1ccc(-c2ccc(-c3ccc4ccc5c(-c6ccc7c(c6)C6(c8ccccc8-c8ccccc86)c6ccccc6-7)ccc6ccc3c4c65)cc2)cc1] | 2.9 | −5.9 | 171 |
| Z-20 | 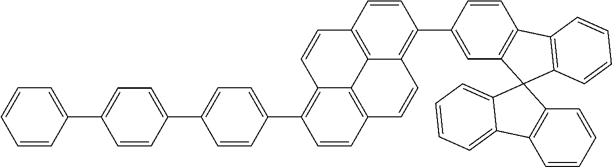 [SMILES: c1ccc(-c2ccc(-c3ccc(-c4ccc5ccc6c(-c7ccc8c(c7)C7(c9ccccc9-c9ccccc97)c7ccccc7-8)ccc7ccc4c5c76)cc3)cc2)cc1] | 2.8 | −5.9 | 173 |

It was found that Tg of Z-3 or Z-19, in which biphenyl derivatives (biphenyl and triphenylene or spirofluorene) were substituted on both sides of pyrene, was improved by about 20° C., compared with Z-4 or Z-18, in which a biphenyl derivative (triphenylene or spirofluorene) was substituted only on one side of pyrene. Therefore, the organic compound represented by general formula [1-1] is preferred as the first organic compound.

Z-18 or Z-19, which was substituted with spirofluorene as a substituent for pyrene, was found to have a Tg improved by about 20° C. or higher as compared with Z-3 or Z-4 substituted with triphenylene. Therefore, the organic compound represented by general formula [1-2] is preferred as the first organic compound. The organic compound represented by general formula [1-3], in which spirofluorene and a biphenyl derivative are substituted, is more preferred.
(ii) The shallow HOMO results in a strong hole-trapping property and improved driving durability.

As described above, shallower HOMO (closer to the vacuum level) results in better hole-trapping property and more improved driving durability characteristics.

As presented in Table 6, it was found that Z-18 or Z-19 substituted with spirofluorene as a substituent for pyrene was found to have a shallower HOMO than Z-3 or Z-4 substituted with triphenylene. Therefore, the organic compound represented by general formula [1-2] is preferred as the first organic compound in terms of a strong hole-trapping property.
(iii) The narrow band gap results in a low injection barrier and a low driving voltage.

In an organic light-emitting device, when charges are injected from a charge injection layer or a charge-blocking layer into a light-emitting layer, a smaller band gap (S1 energy) results in a smaller injection barrier and a lower driving voltage. Furthermore, the smaller injection barrier improves charge accumulation at the interface between the light-emitting layer and an adjacent layer, and also improves the driving durability characteristics.

As presented in Table 6, Z-3 or Z-19, in which the biphenyl derivatives are substituted on both sides of pyrene, was found to have smaller S1 than Z-4 or Z-18, in which the biphenyl derivative is substituted on only one side of pyrene. Therefore, the organic compound represented by general formula [1-1] is preferred as the first organic compound in terms of reducing the voltage. By changing the biphenyl group substituted on one side of pyrene in Z-19 to a terphenyl group as in Z-20, S1 is even smaller, which is more preferred in terms of reducing the voltage.

[7] The material of the hole-blocking layer has a fused-ring skeleton composed of a hydrocarbon.

As described in [3] above, typically, an electron-blocking material has a freely rotatable sp² carbon-nitrogen bond and is electron-rich in a neutral state; hence, the material is easily decomposed by receiving electrons (reduction) or in an excited state. For this reason, when an electron-blocking layer or a hole-blocking layer is disposed in contact with the light-emitting layer, a recombination zone is preferably localized close to the interface between the hole-blocking layer and the light-emitting layer. In addition, the material of the hole-blocking layer preferably has a stable molecular structure. For example, a skeleton, such as a nitrogen-containing heterocycle, is electron-deficient in a neutral state and thus is easily decomposed when an electron is extracted. In other words, the material is easily decomposed by holes leaked from the light-emitting layer. Therefore, a fused-ring skeleton composed of a hydrocarbon, which is stable even if an electron is extracted (oxidized), is preferred because good durability characteristics are exhibited.

The hole-blocking layer also functions as an electron transport layer and thus preferably has high electron mobility. Thus, among fused-ring skeletons composed of hydrocarbons, those having a fused-ring structure with four or more rings are preferred. This is because the electron mobility improves as the planarity increases.

Specific examples of the first to third organic compounds will be described below.

### Third Organic Compound

In the present invention, the third organic compound used as the host of the light-emitting layer is not particularly limited, except that all of the freely rotatable single bonds are carbon-carbon bonds, preferably sp² carbon-sp² carbon bonds. Preferably, the compound has any one of an anthracene skeleton, a phenanthroline skeleton, a pyrene skeleton, a chrysene skeleton, a benzophenanthrene skeleton, a triphenylene skeleton, a fluoranthene skeleton, a benzochrysene skeleton, and a benzofluoran skeleton. Preferably, the third compound is an organic compound represented by the following general formula [2].

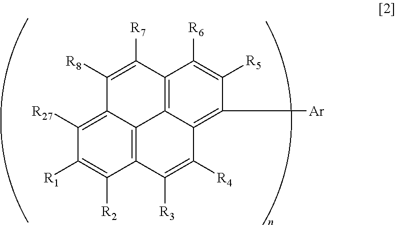

In general formula [2], R₁ to R₈ and R₂₇ are each independently selected from a hydrogen atom, a deuterium atom, a substituted or unsubstituted alkyl group, a substituted or unsubstituted aryl group, and a substituted or unsubstituted heterocyclic group. n is a natural number selected from 1 to 3.

Specific examples of the third organic compound are illustrated below, but the third organic compound is not

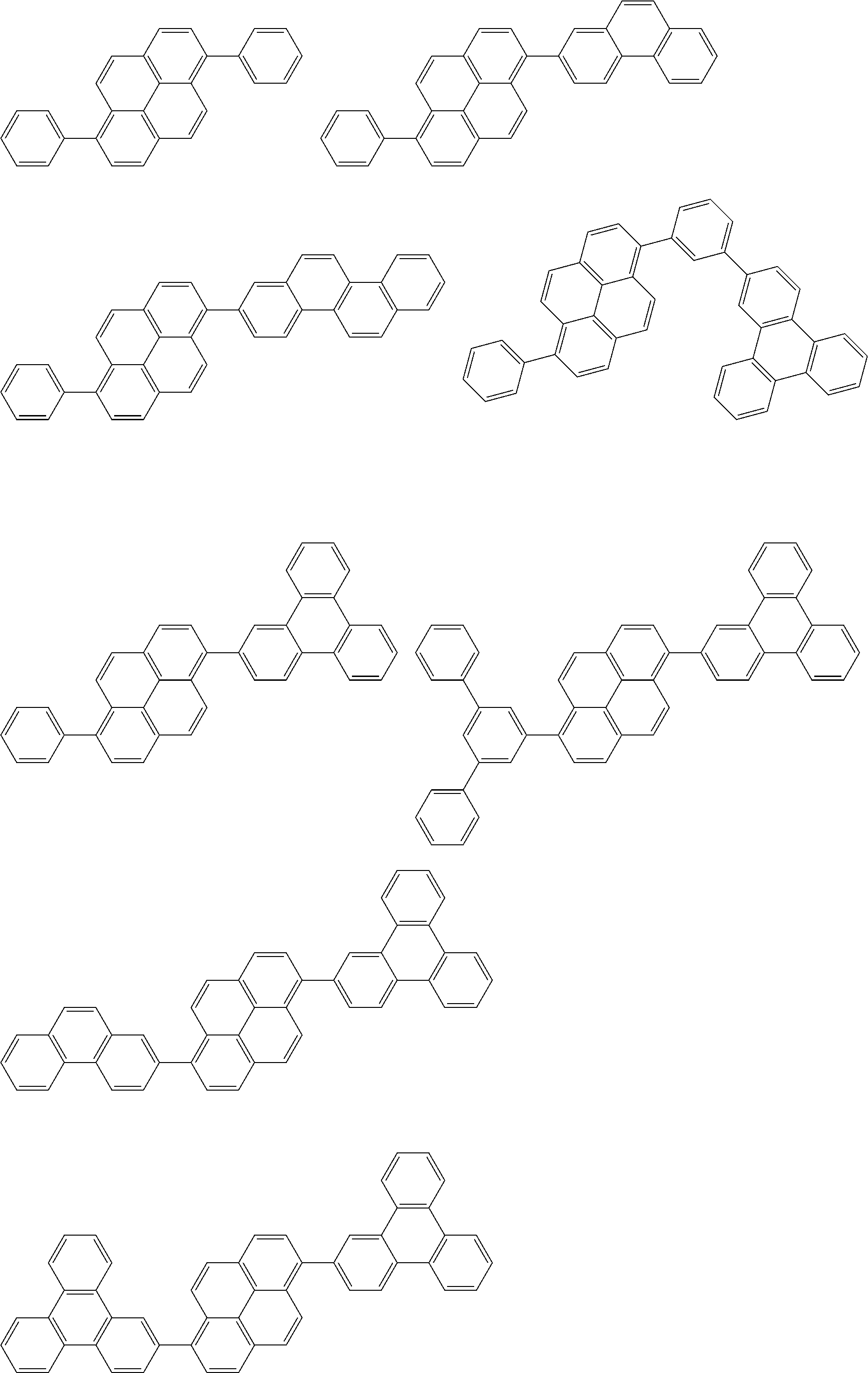

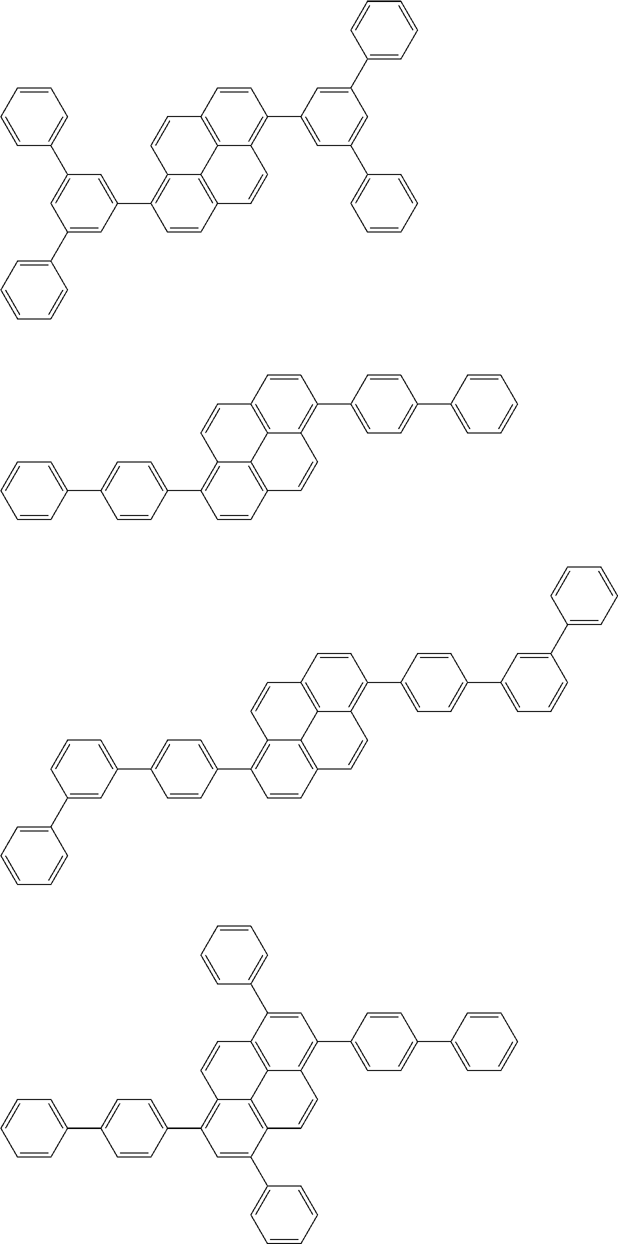

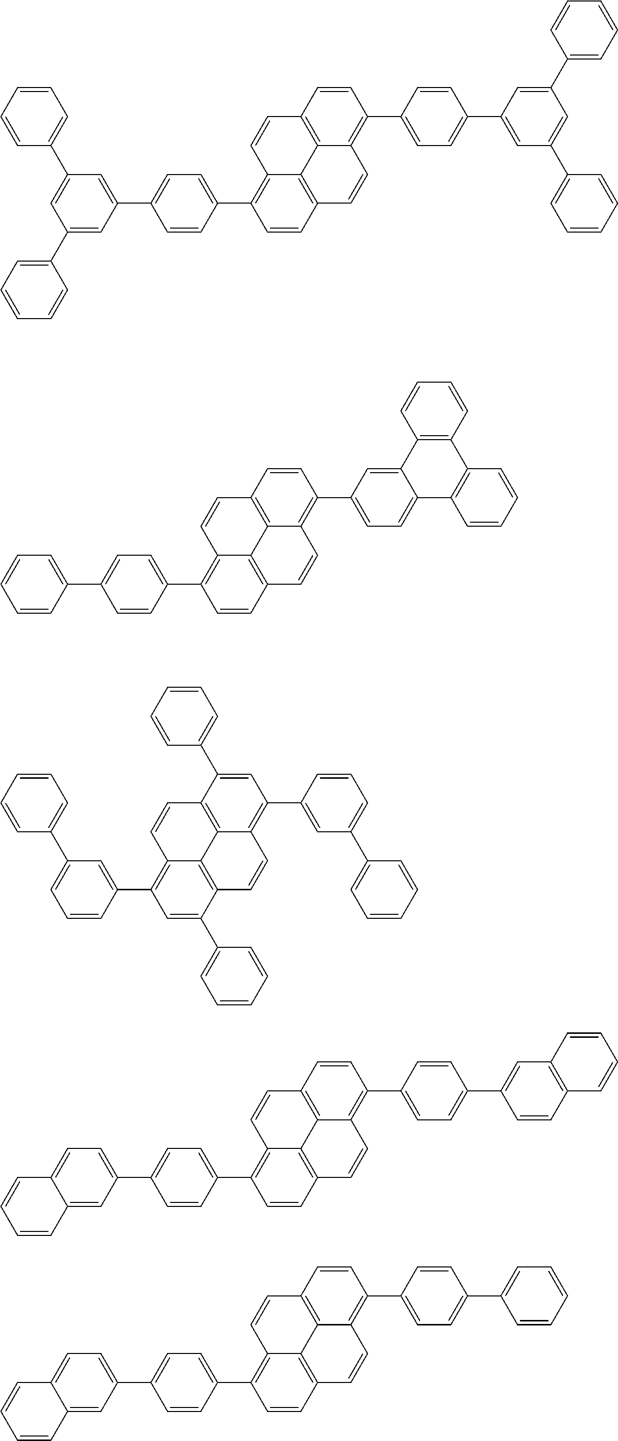

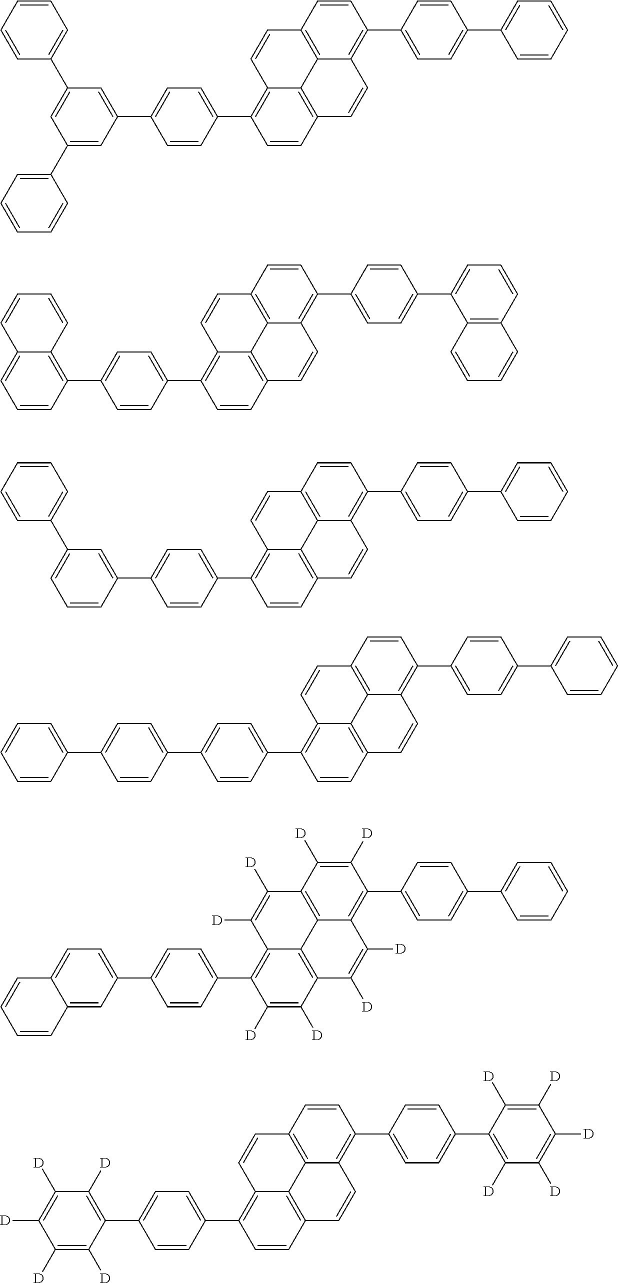

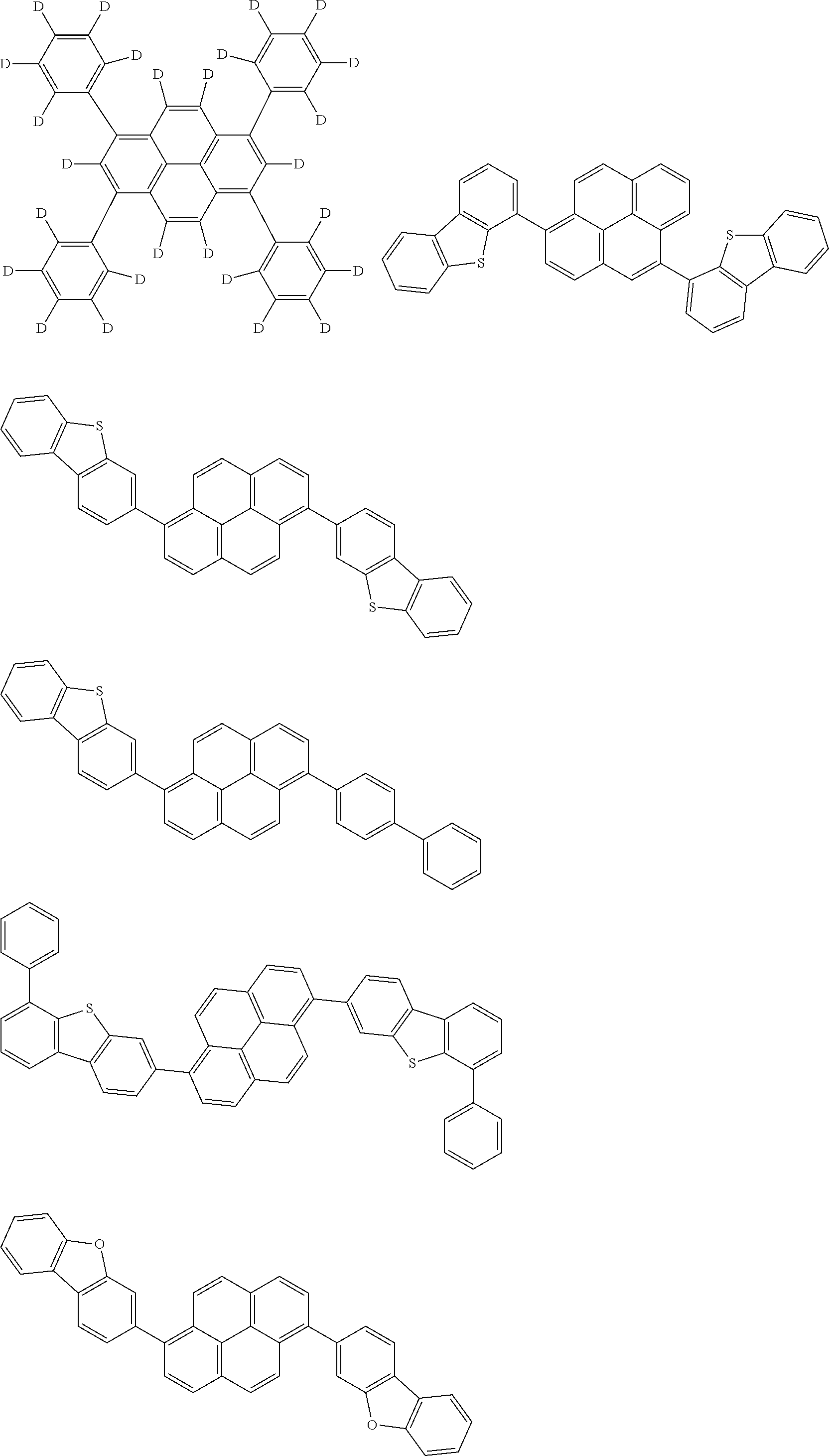

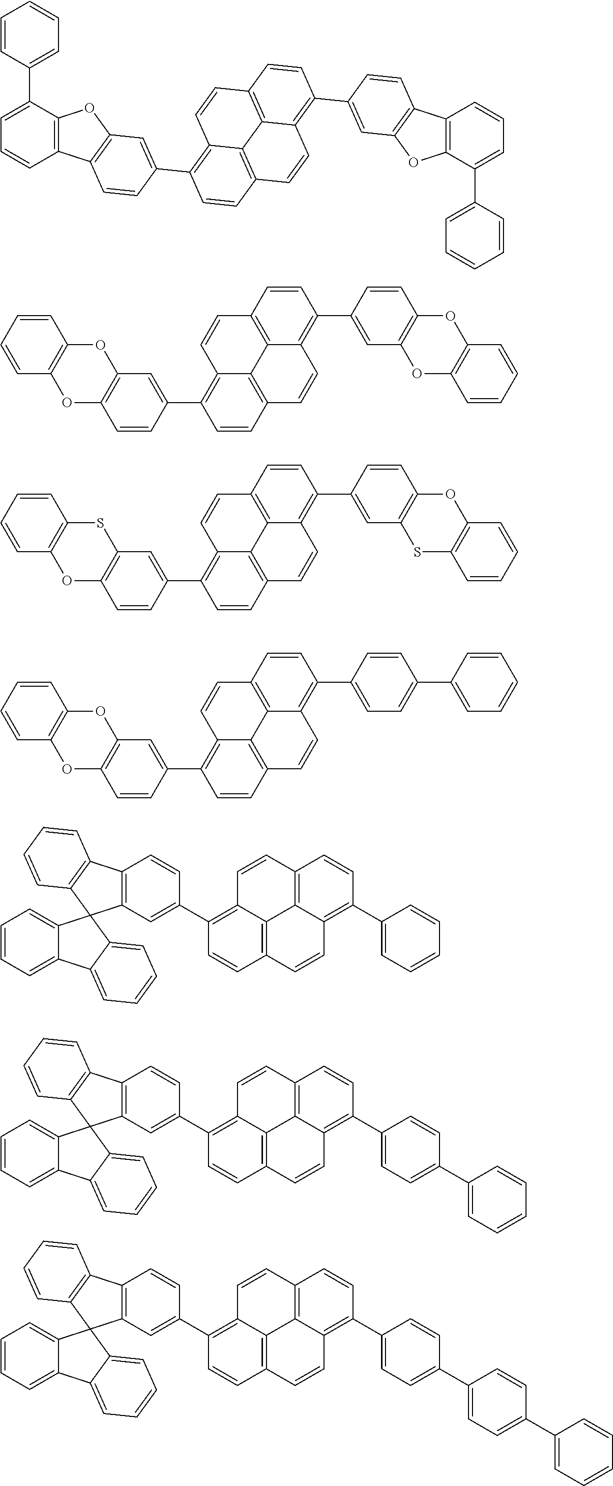

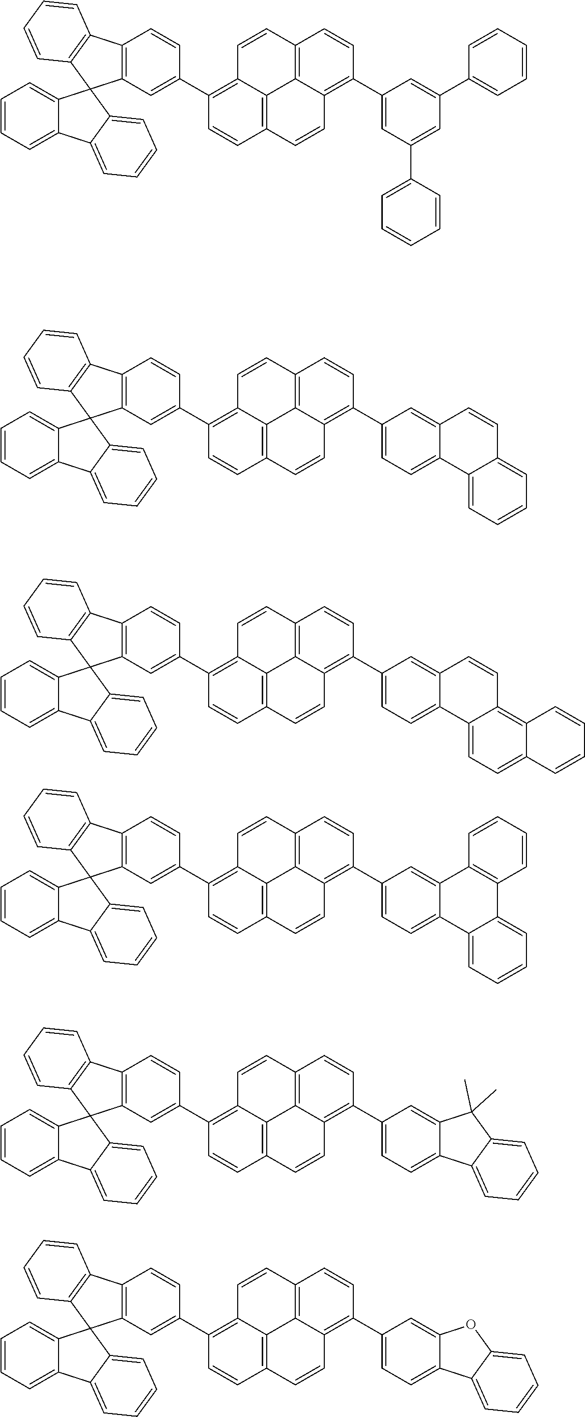

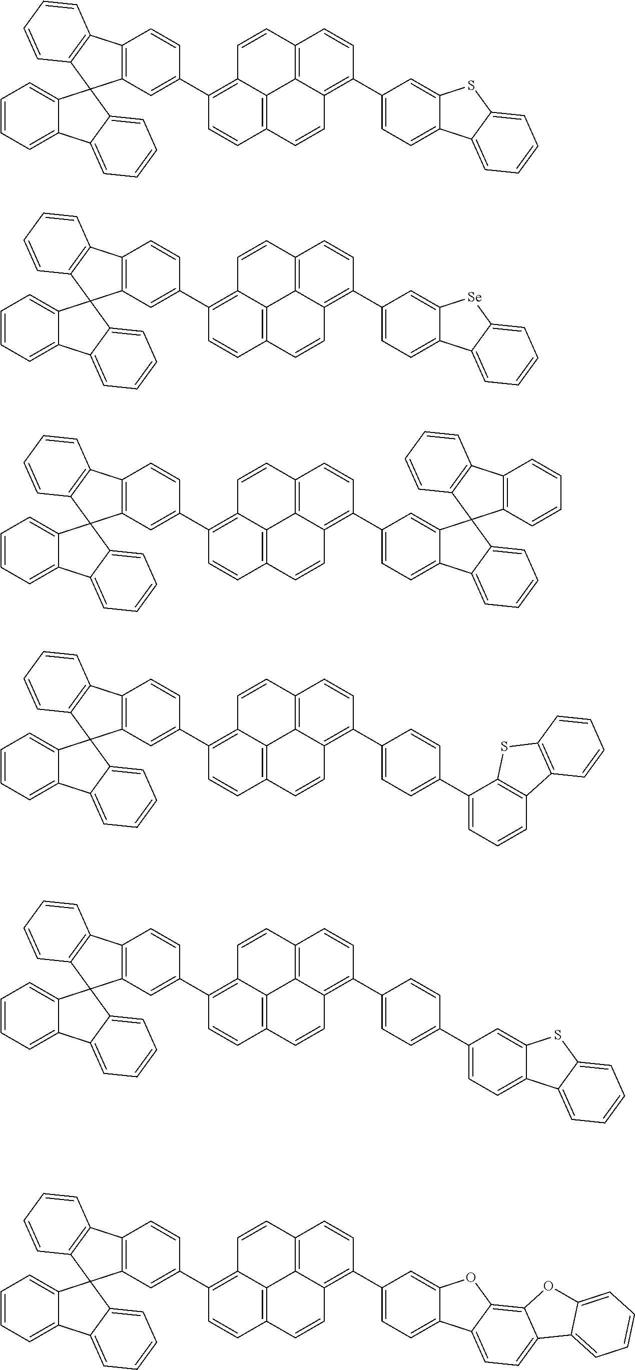

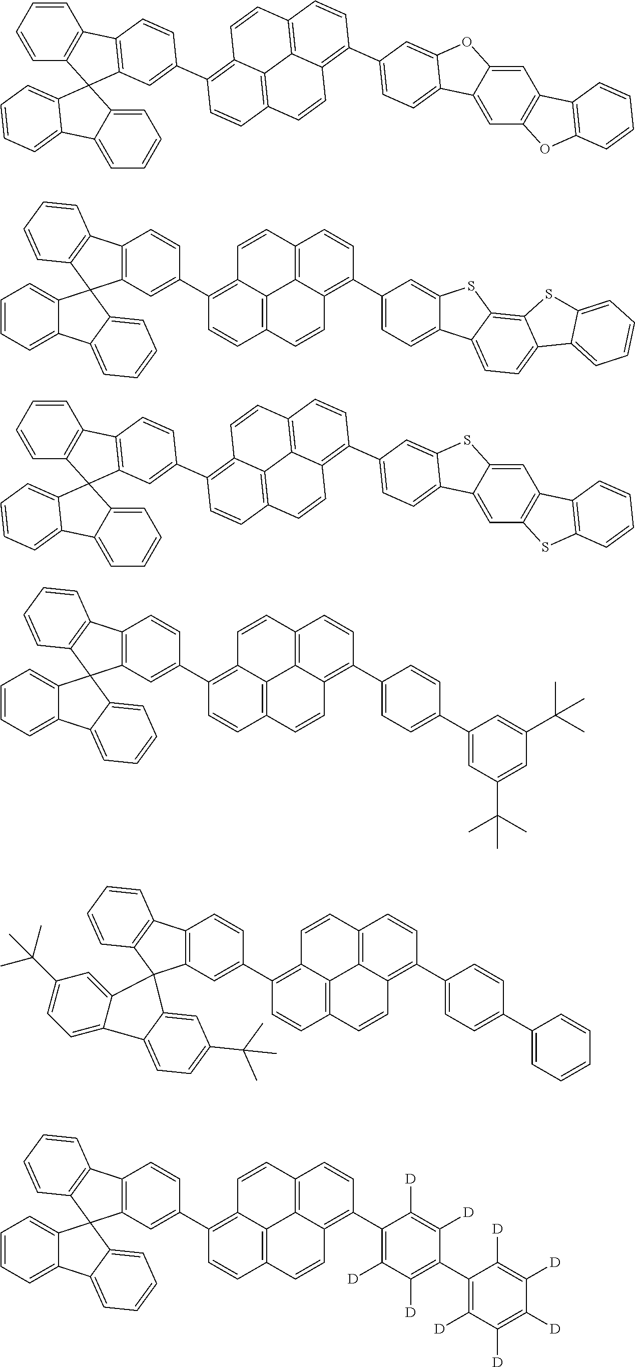

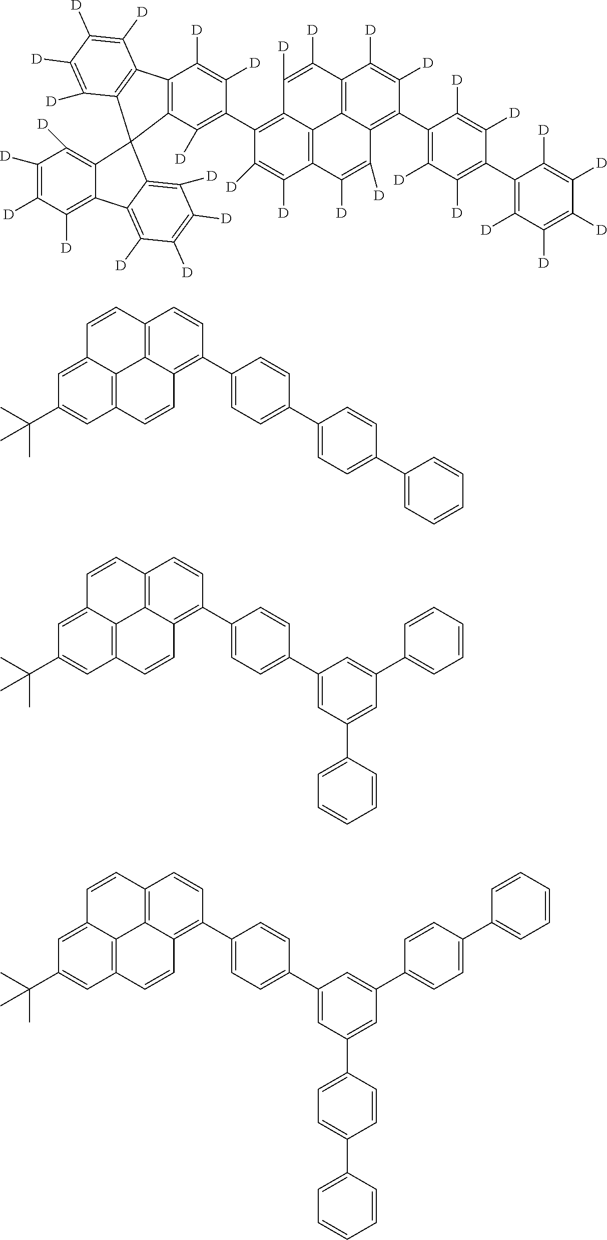

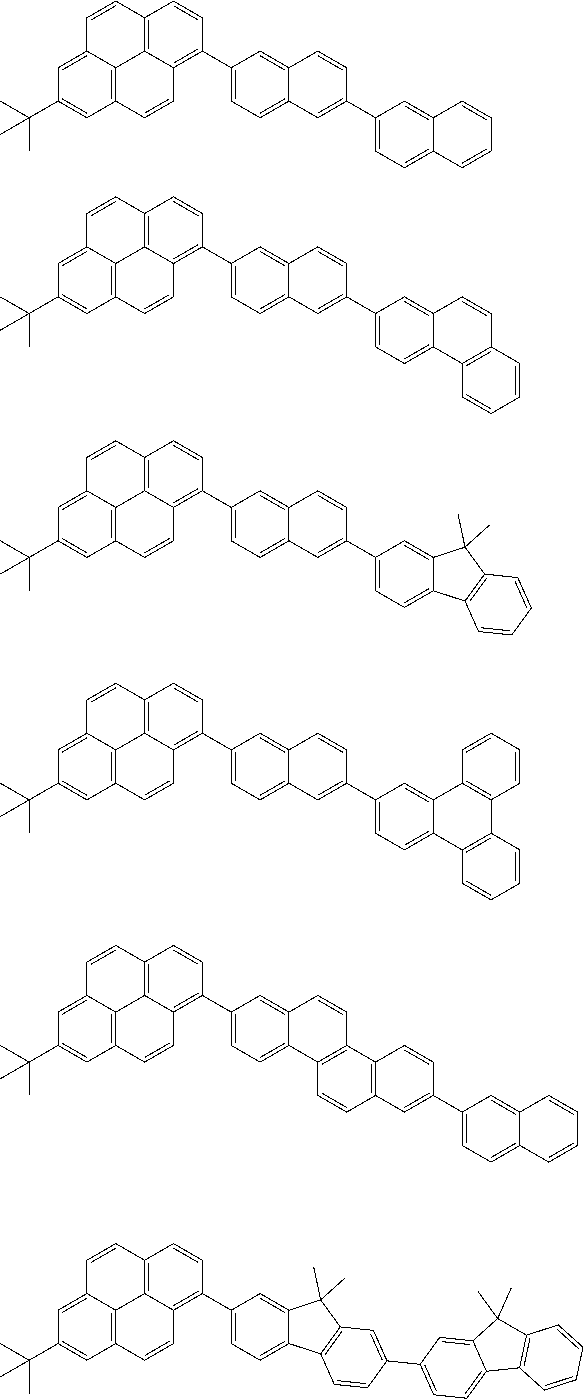

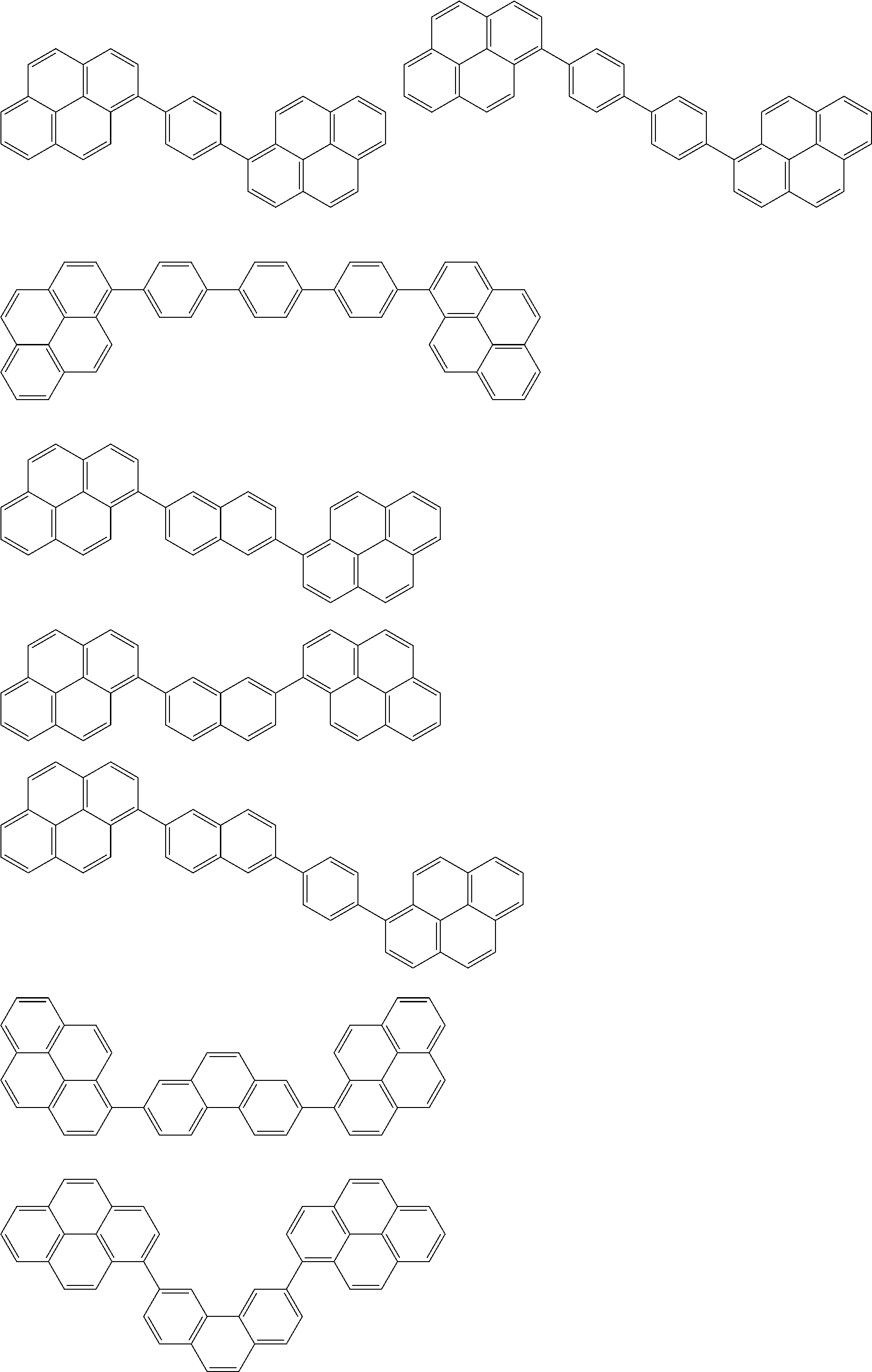

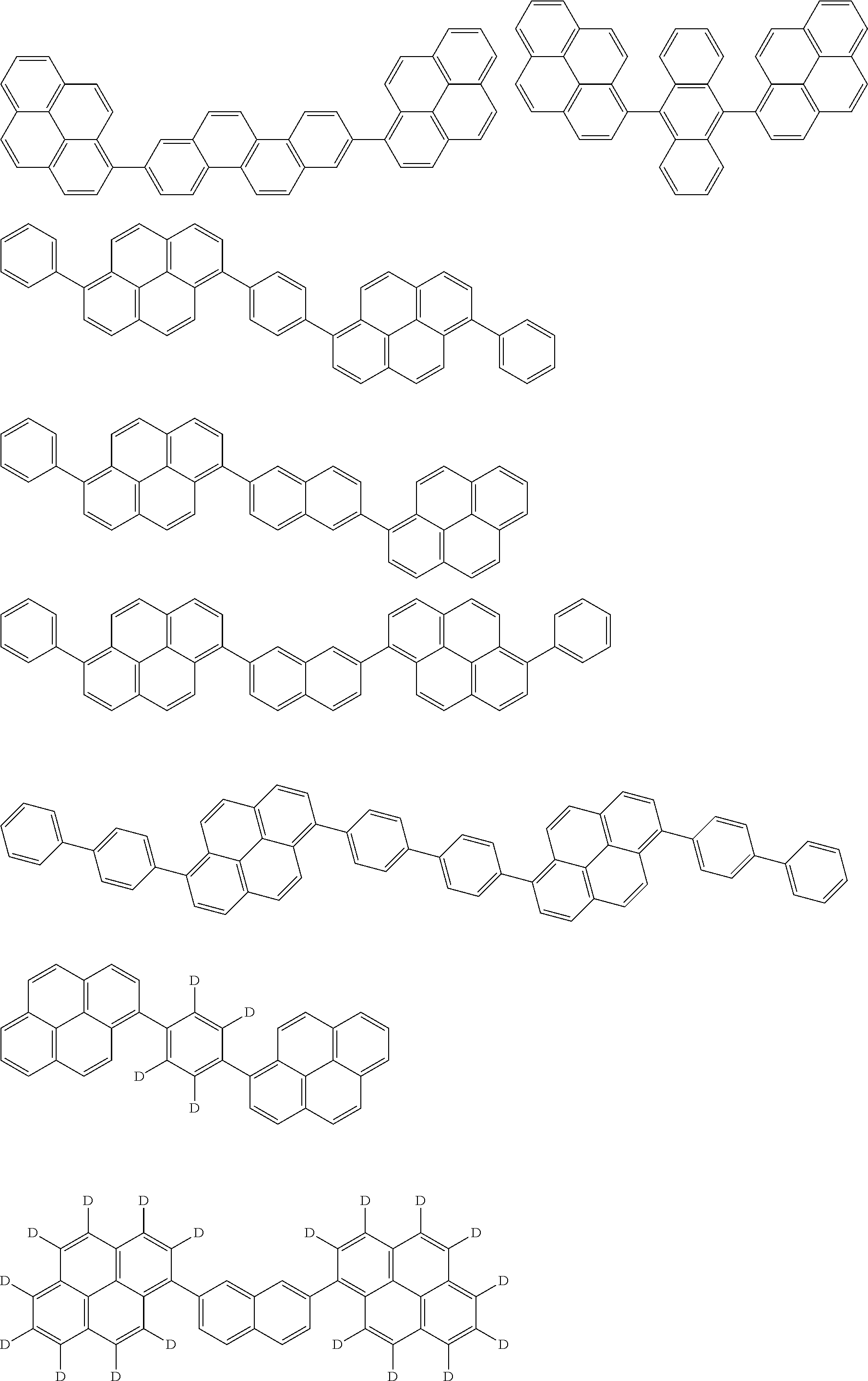

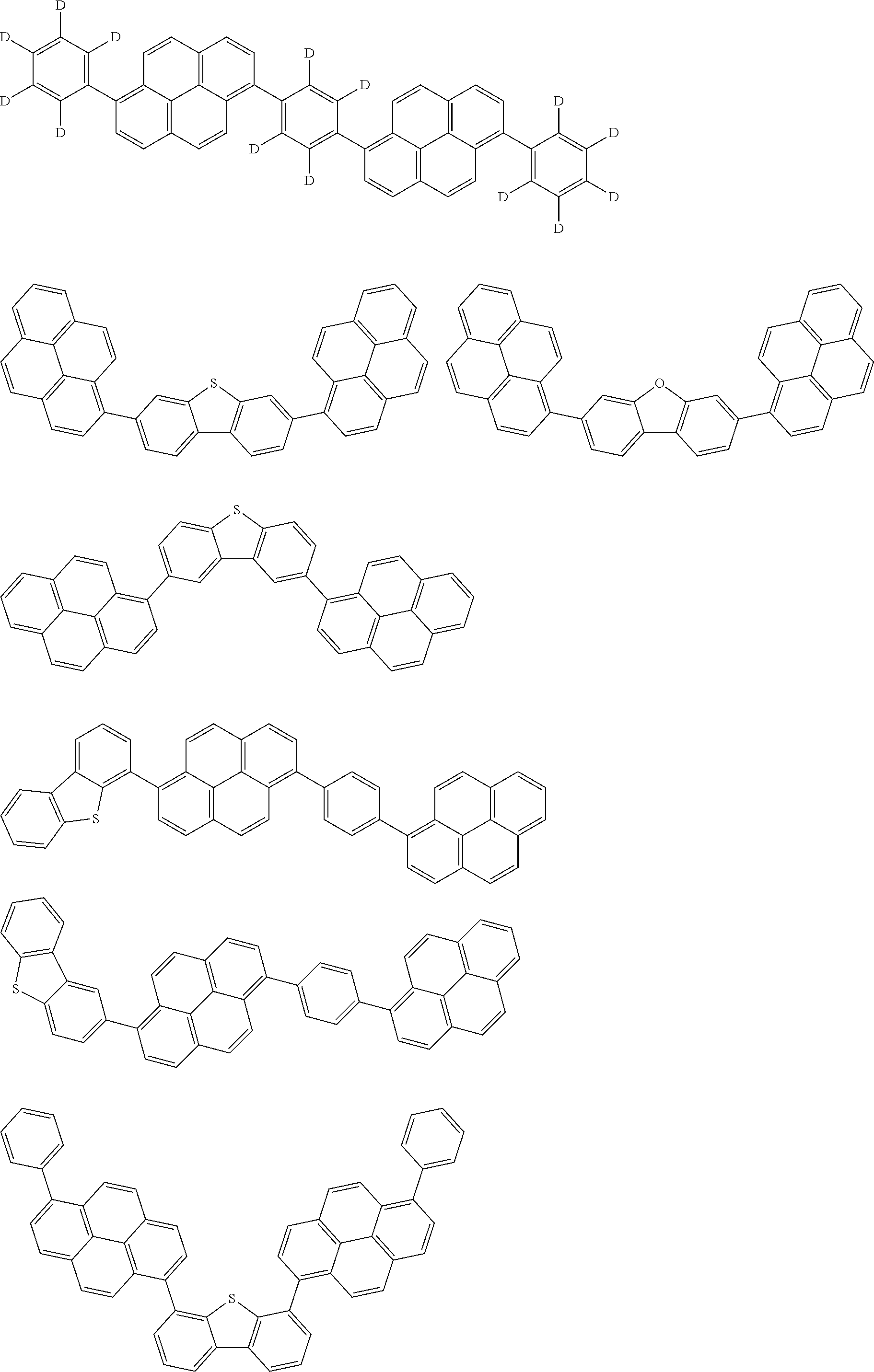

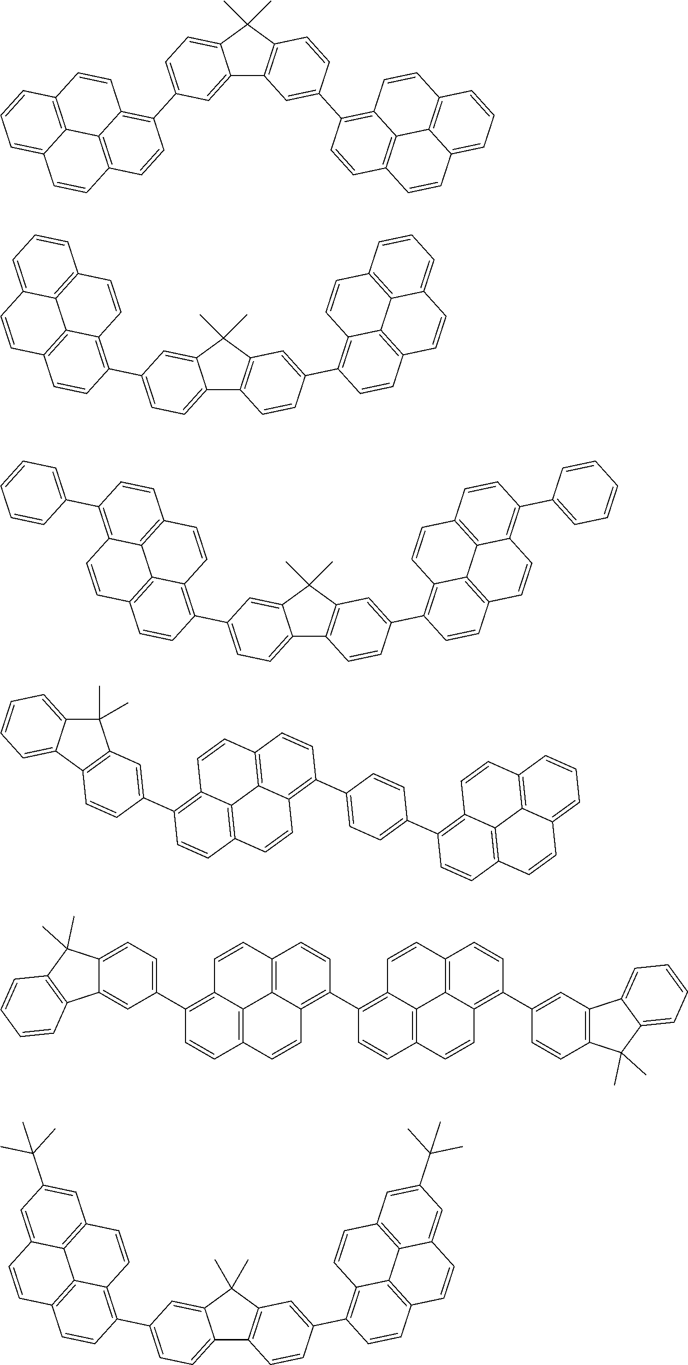

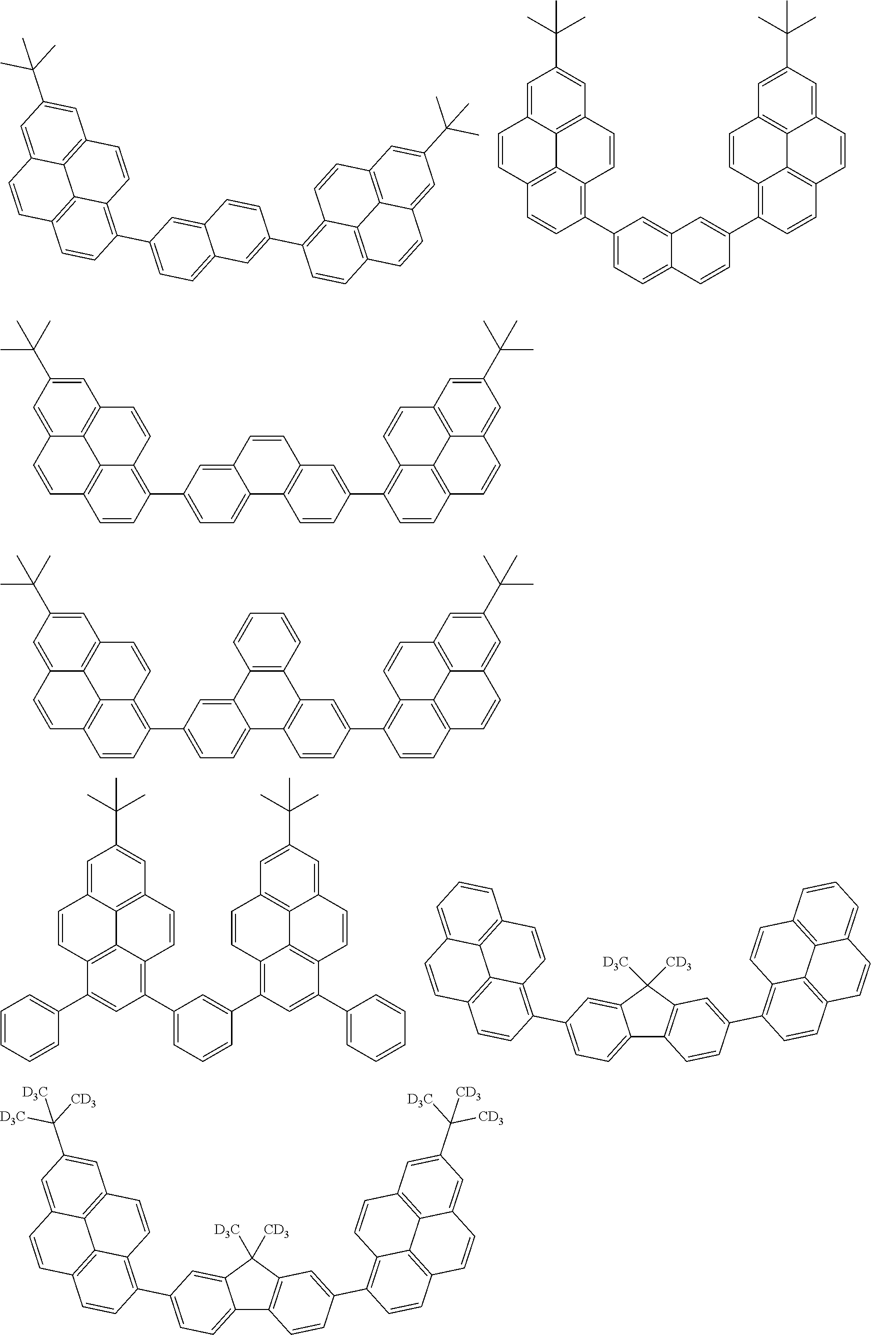

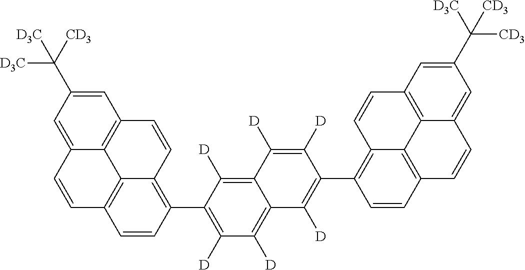 [SMILES: [2H]c1c(-c2ccc3ccc4cc(C(C([2H])([2H])[2H])(C([2H])([2H])[2H])C([2H])([2H])[2H])cc5ccc2c3c45)c([2H])c2c([2H])c([2H])c(-c3ccc4ccc5cc(C(C([2H])([2H])[2H])(C([2H])([2H])[2H])C([2H])([2H])[2H])cc6ccc3c4c56)c([2H])c2c1[2H]]

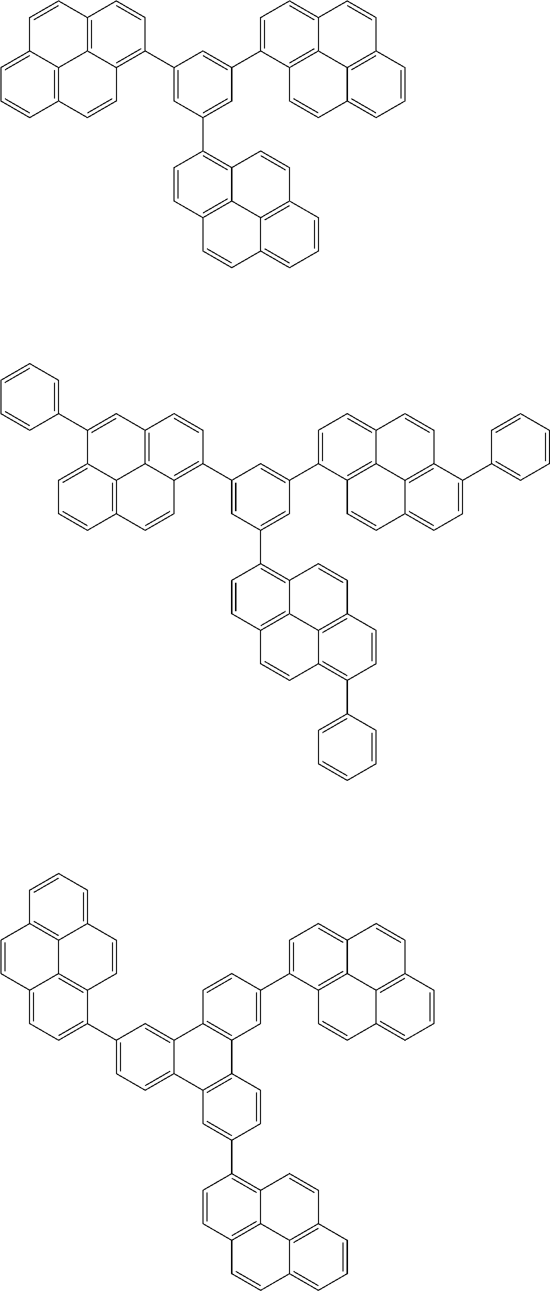

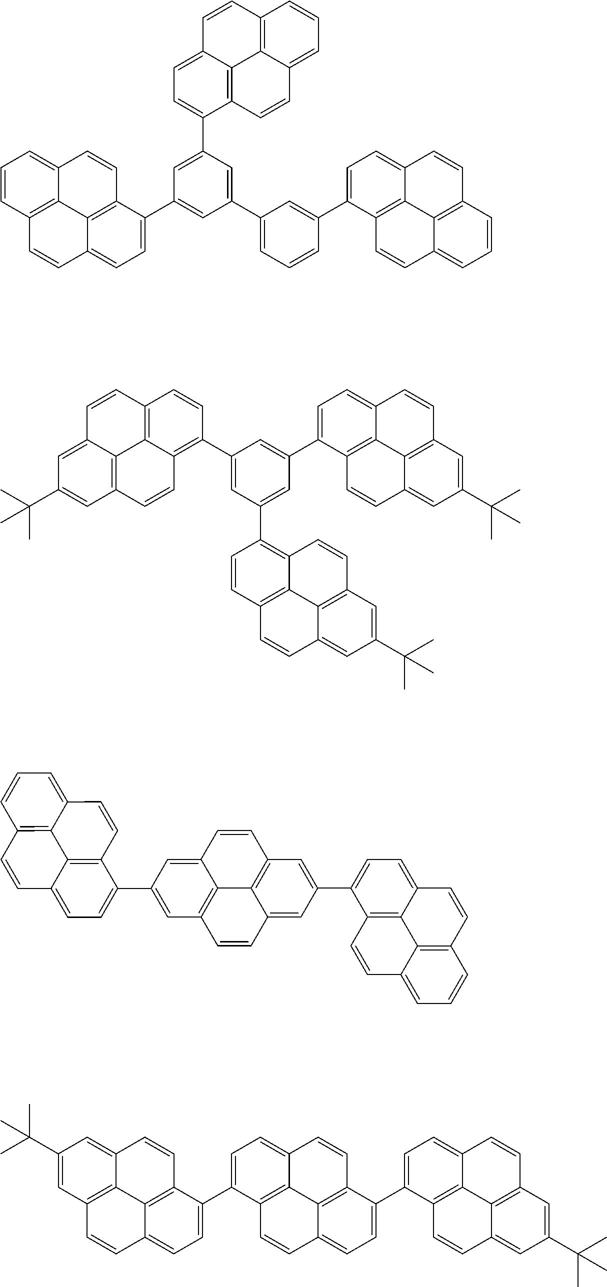

### First Organic Compound

The first organic compound is not particularly limited, except that all of the freely rotatable single bonds are carbon-carbon bonds, preferably sp² carbon-sp² carbon bonds. Preferred examples thereof include the organic compound represented by general formula [1] above, the organic compound represented by general formula [2] given as a preferred example of the third organic compound, and organic compounds having partial skeletons represented by the following general formulae [3-1] to [3-4].

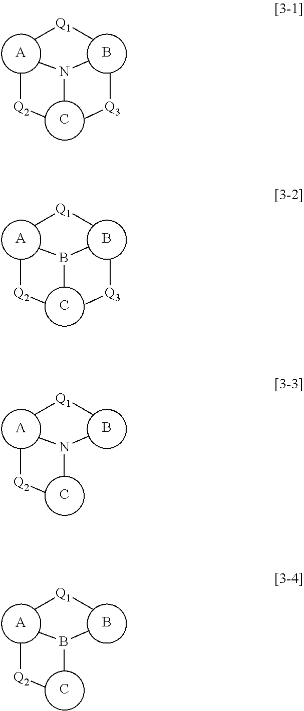

In general formulae [3-1] to [3-4], cyclic units A to C are each an aromatic hydrocarbon group or a heterocyclic group and may each be a fused ring. Q₁ to Q₃ are each independently selected from a direct bond, CR₁R₂, NR₃, an oxygen atom, a sulfur atom, a selenium atom, and a tellurium atom. Substituents R₁ to R₃ are each independently selected from a substituted or unsubstituted alkyl group, a substituted or unsubstituted alkoxy group, a substituted or unsubstituted aryl group, and a substituted or unsubstituted heteroaryl group. R₃ may form a ring together with any one of adjacent cyclic units A to C.

Preferred specific examples of the first organic compound represented by general formula [1] are illustrated below, but the first organic compound is not limited thereto.

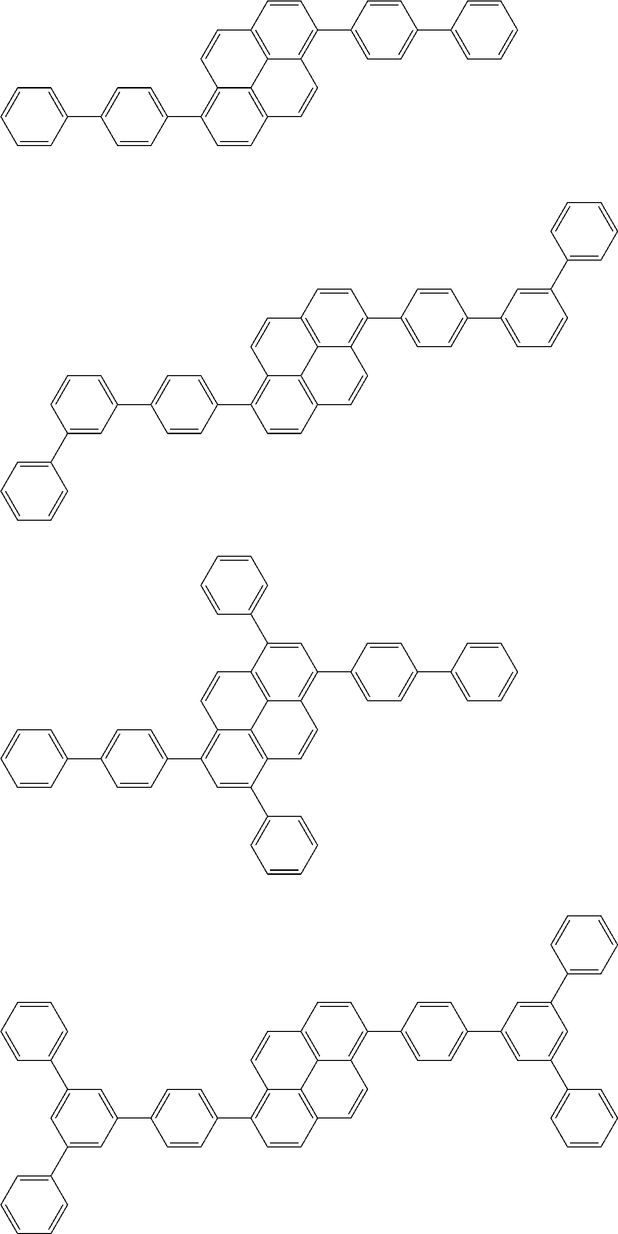

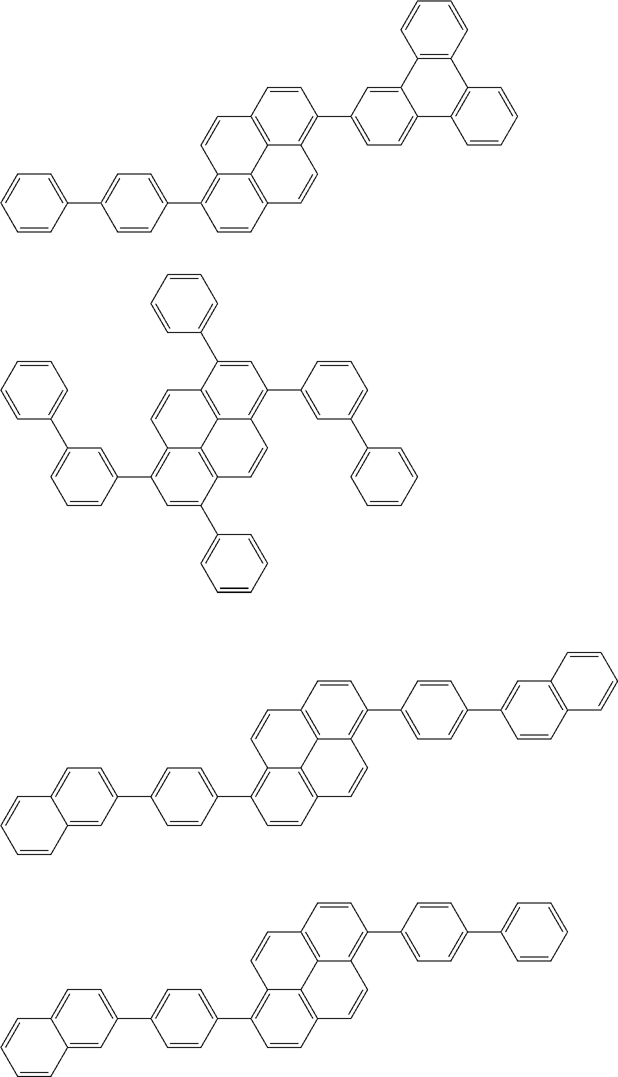

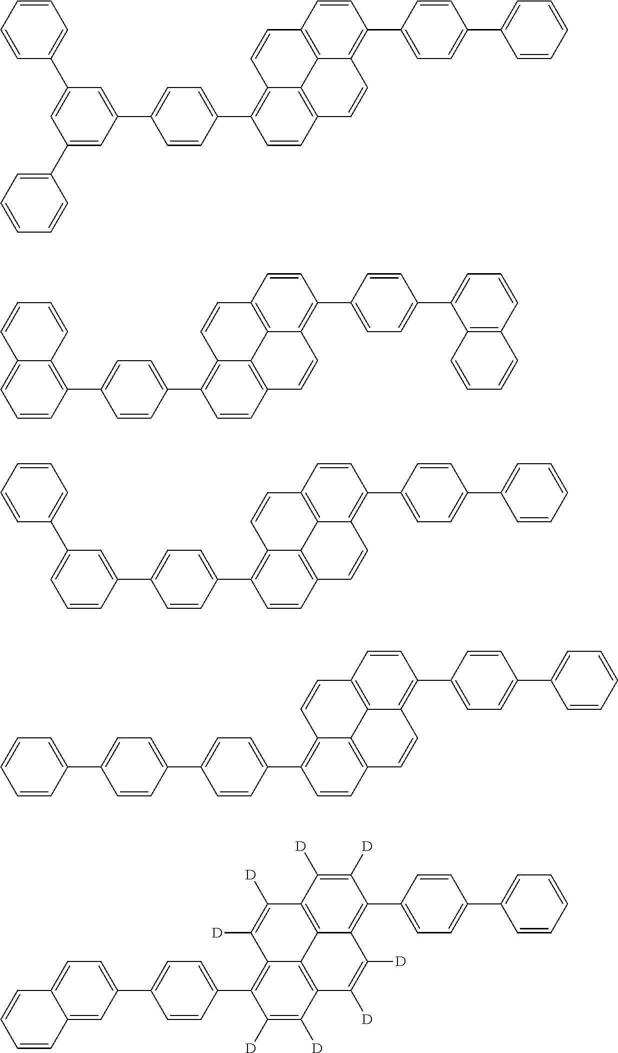

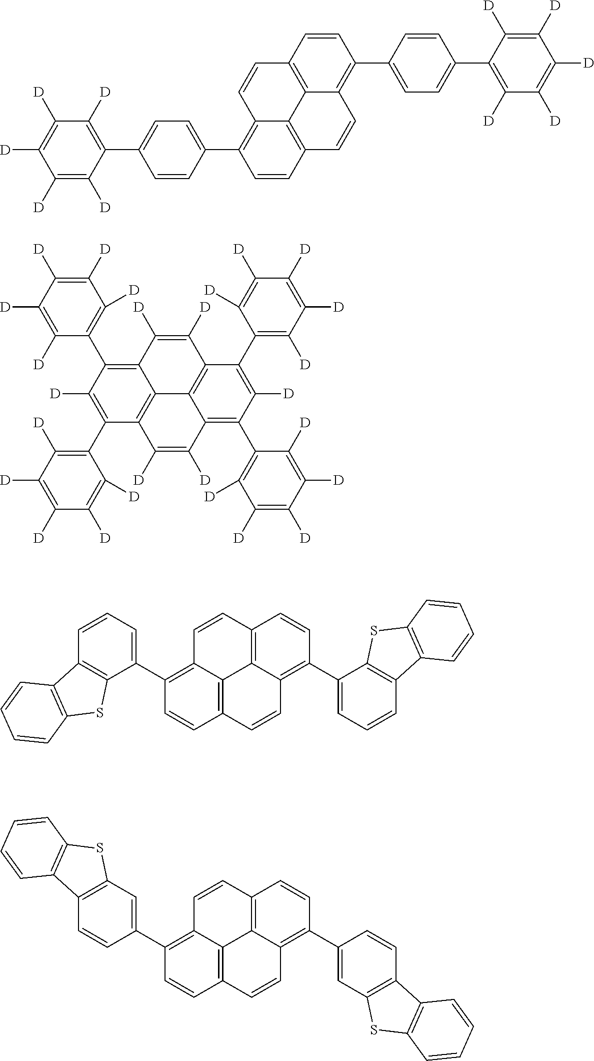

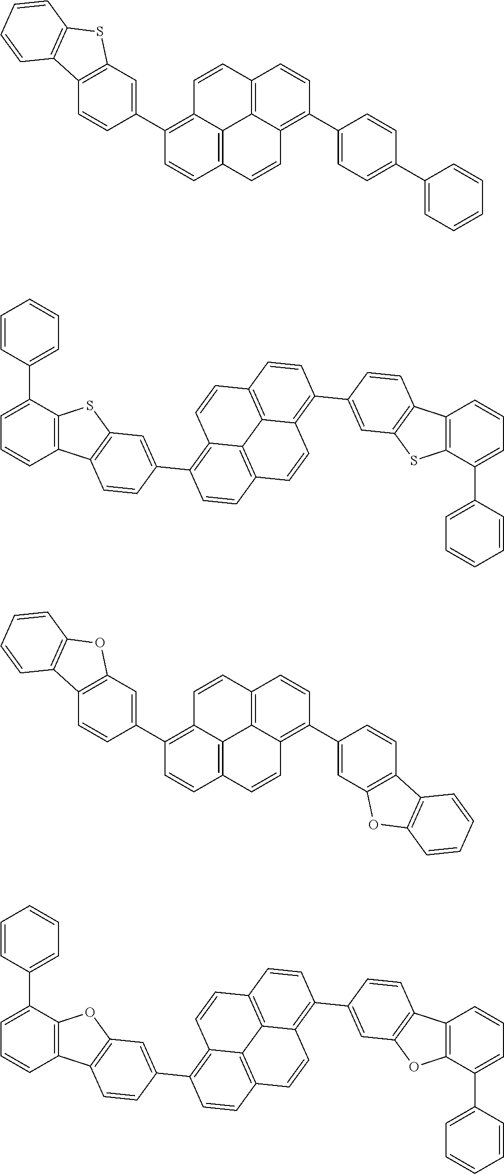

Preferred specific examples of the first organic compound represented by general formula [2] are illustrated below, but the first organic compound is not limited thereto.

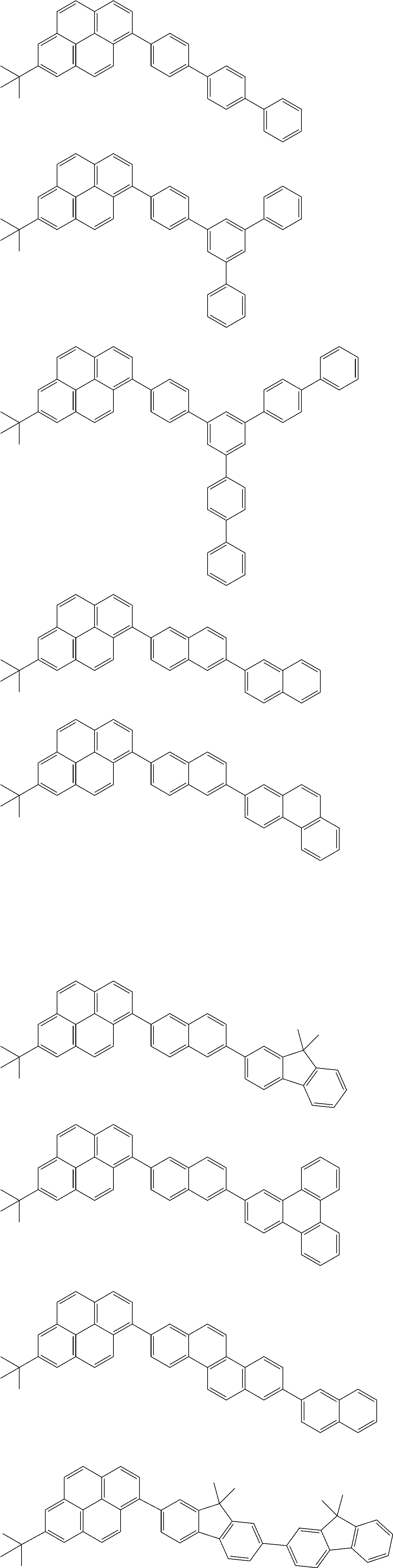

Specific examples of the partial skeletons represented by general formulae [3-1] to [3-4] are illustrated below, but the partial skeletons are not limited thereto.

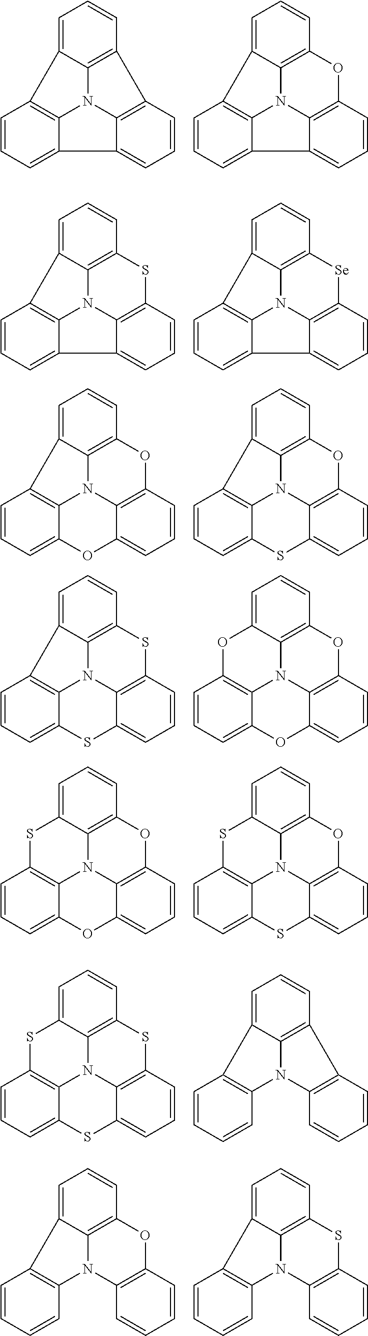

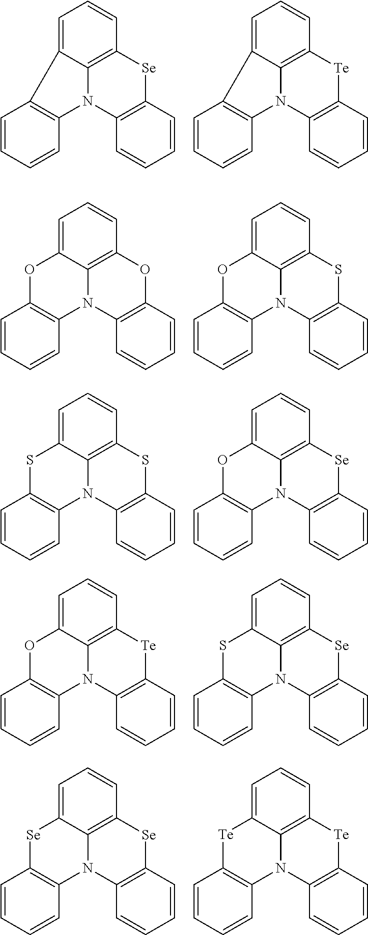

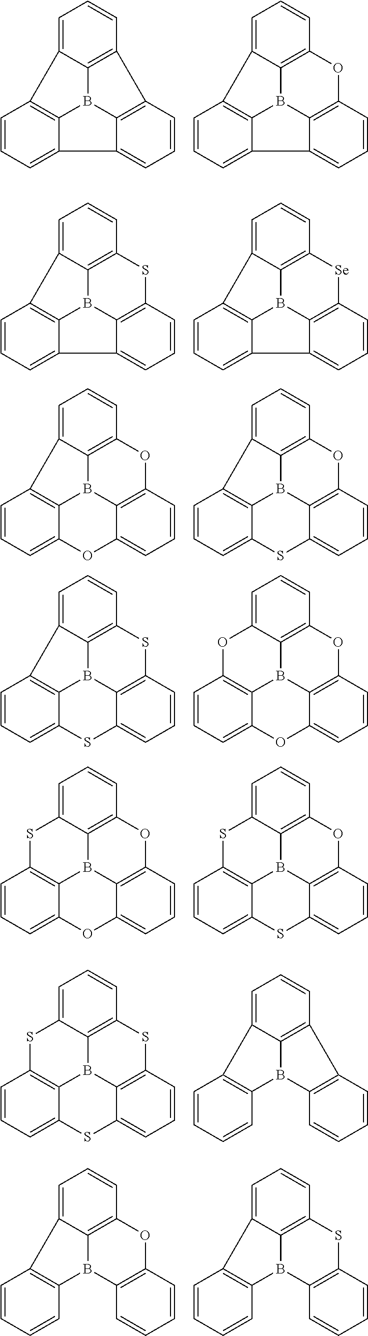

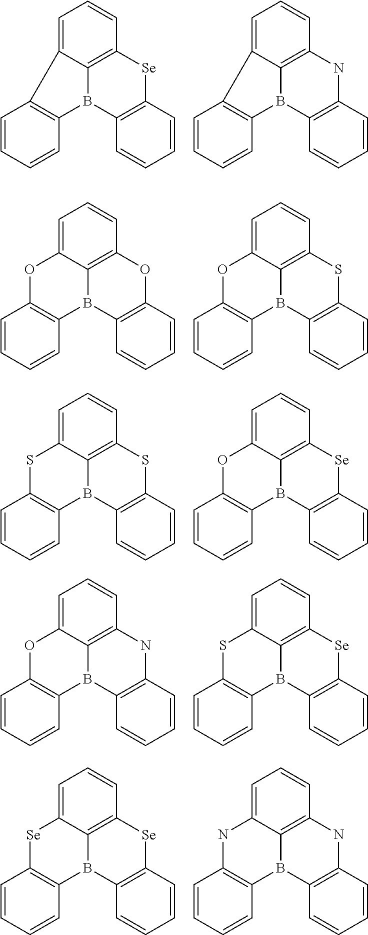

Specific examples of the first organic compound having a partial skeleton represented by any one of general formulae [3-1] to [3-4] are illustrated below, but the first organic compound is not limited thereto.

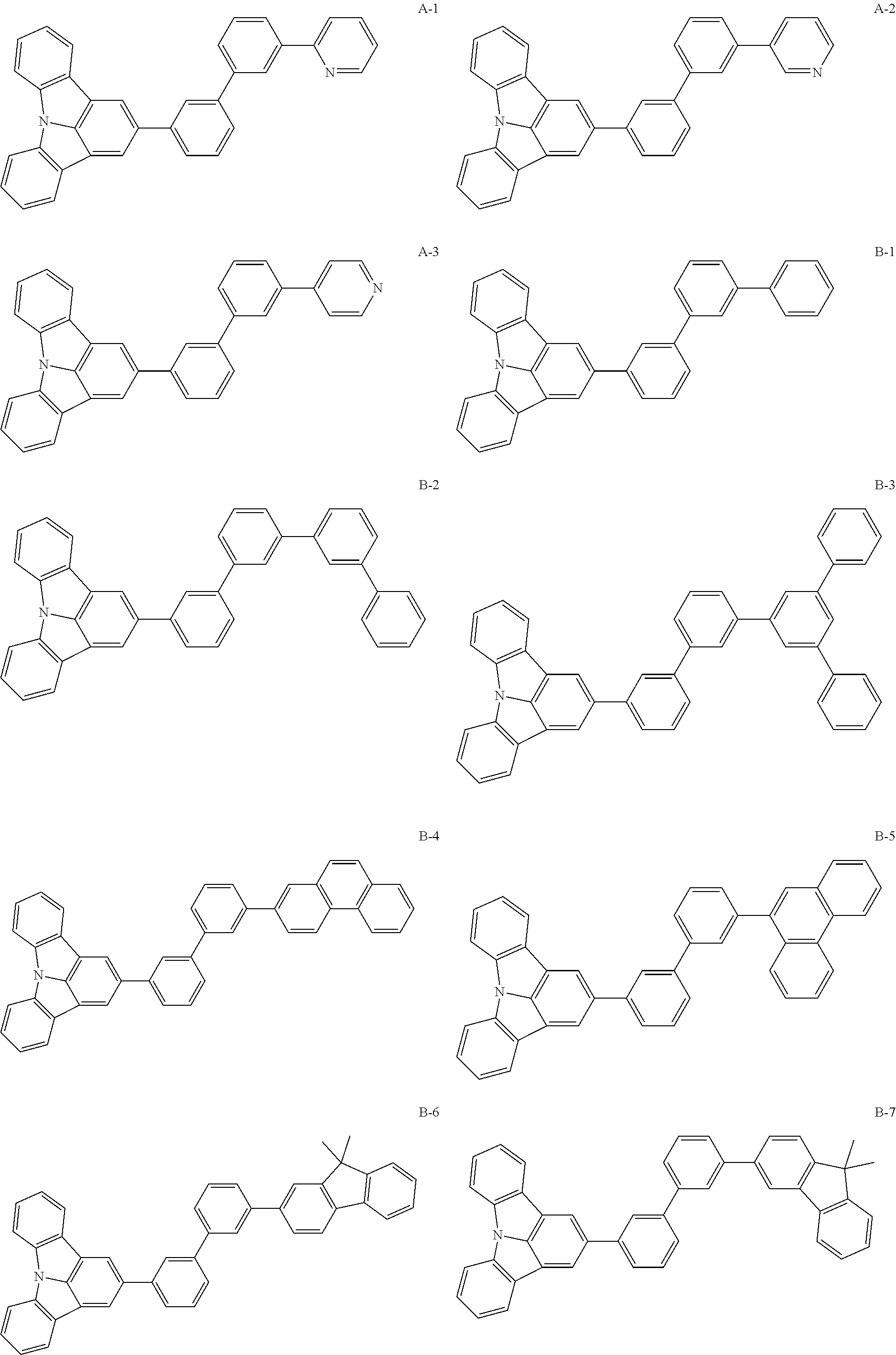

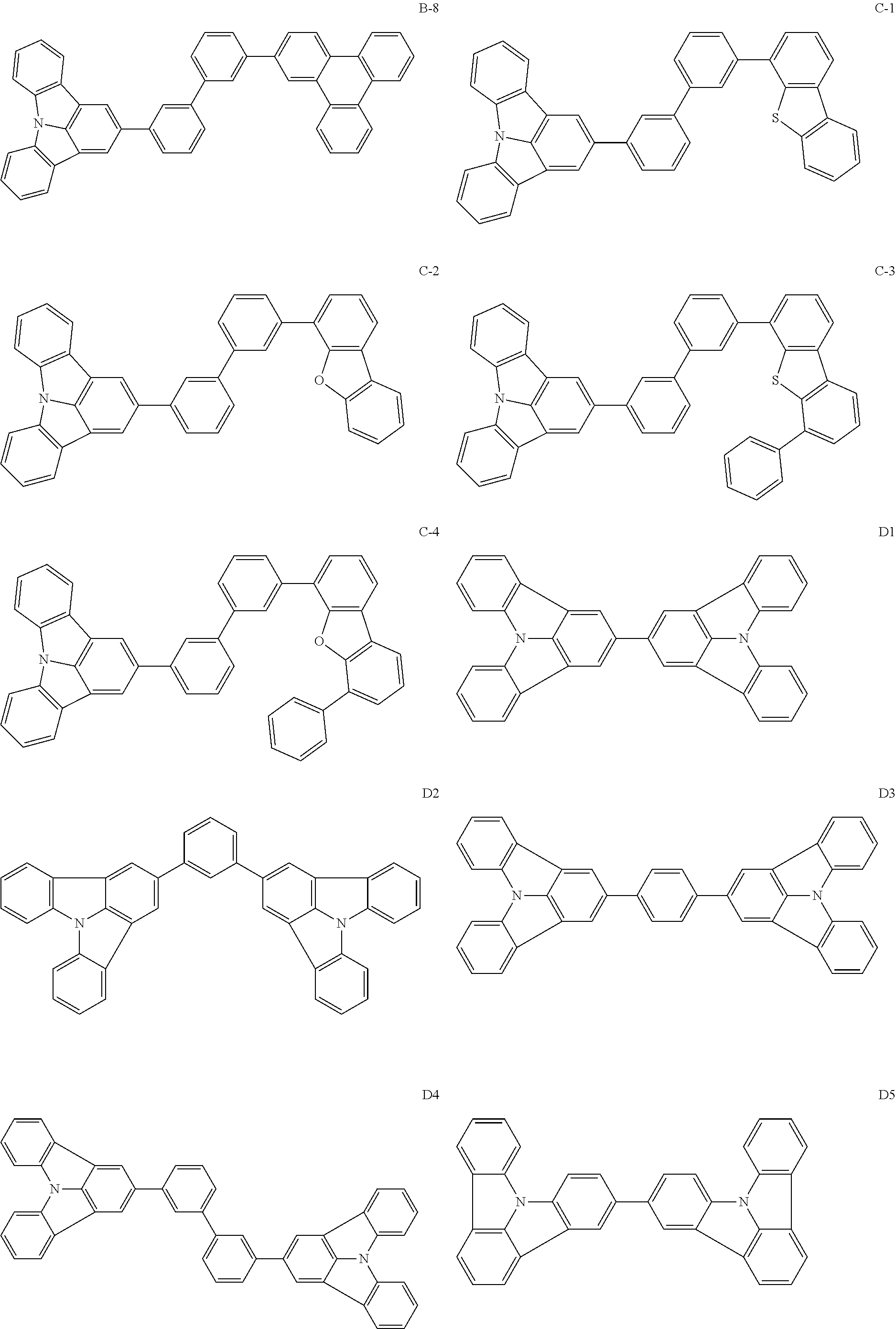

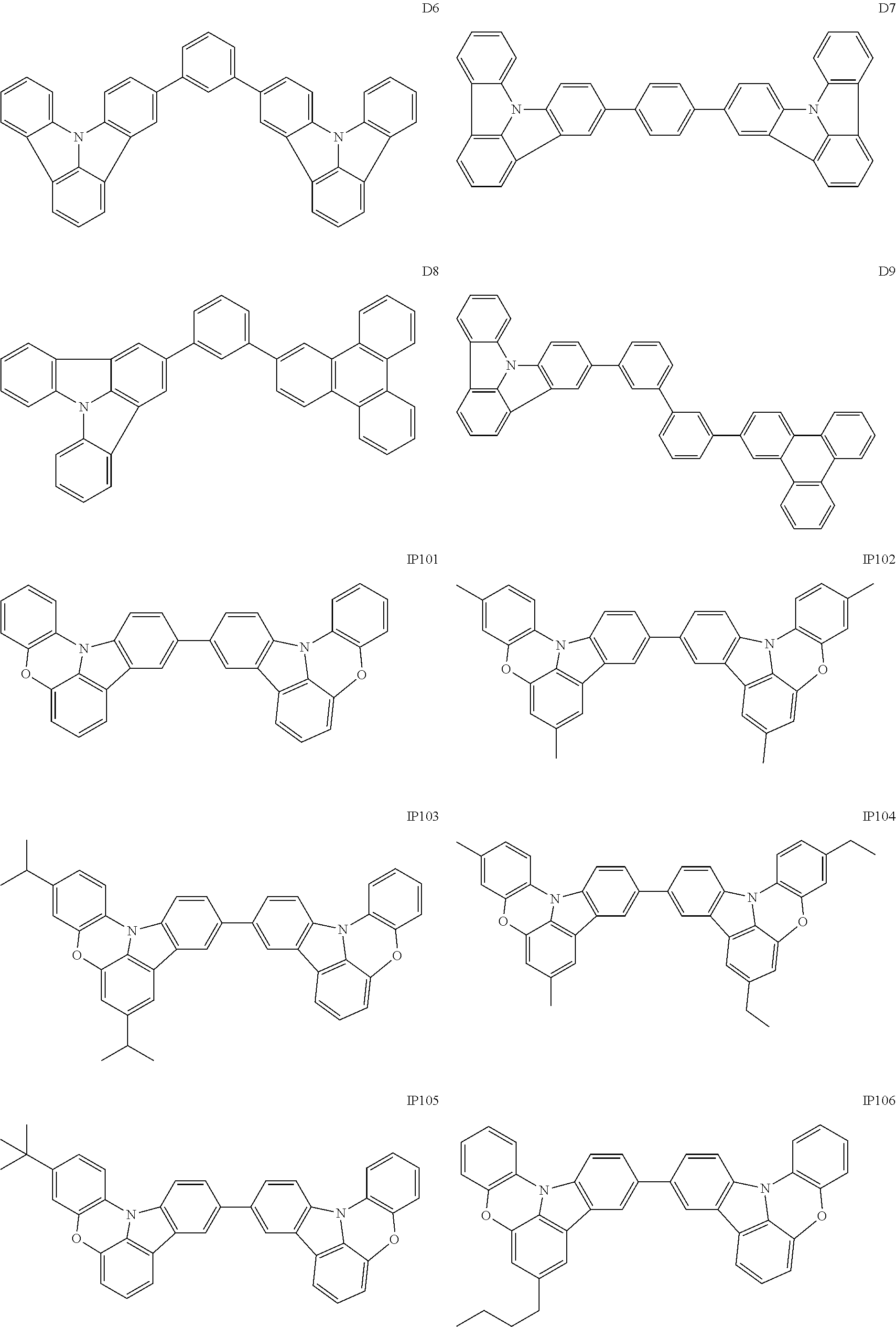

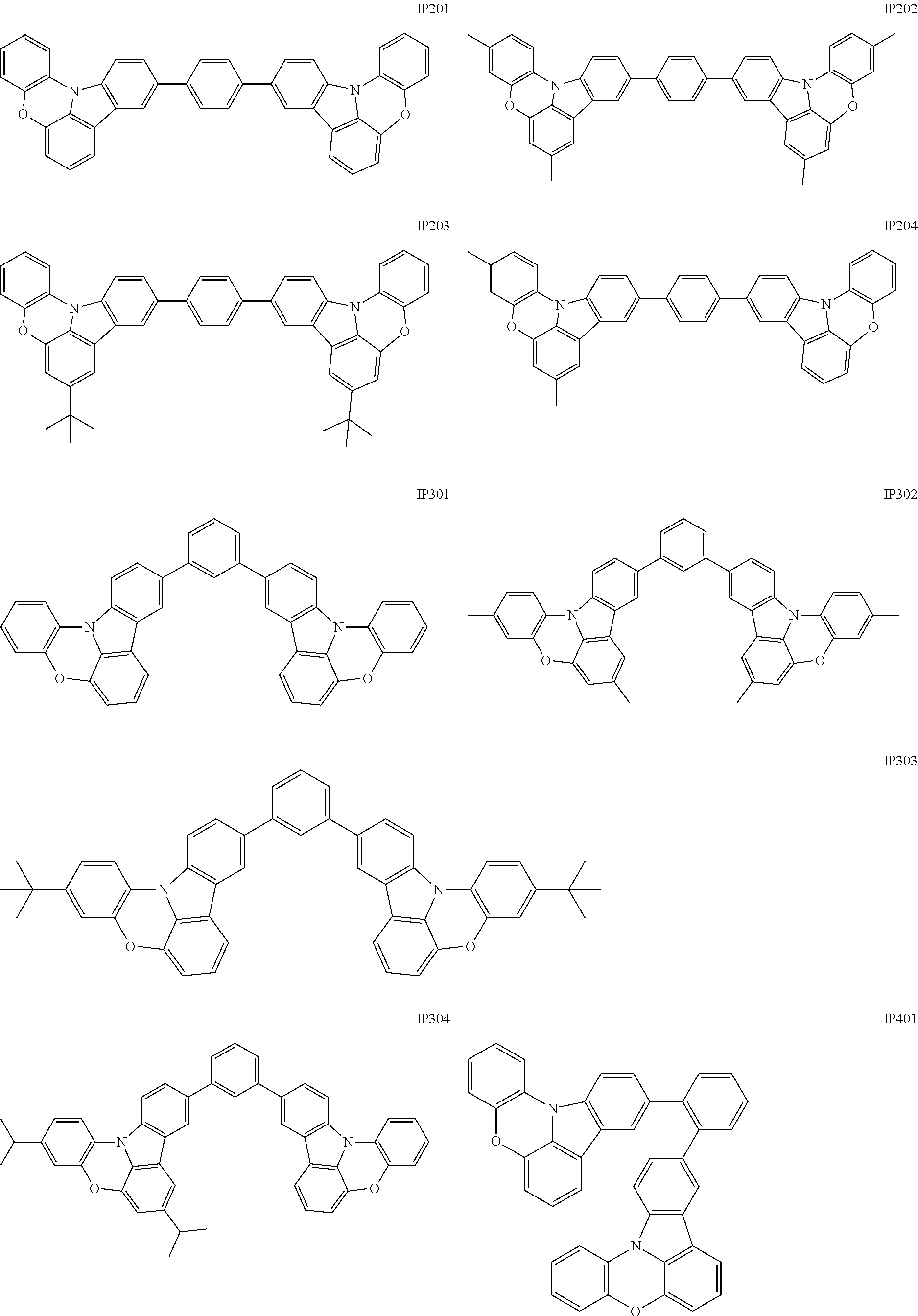

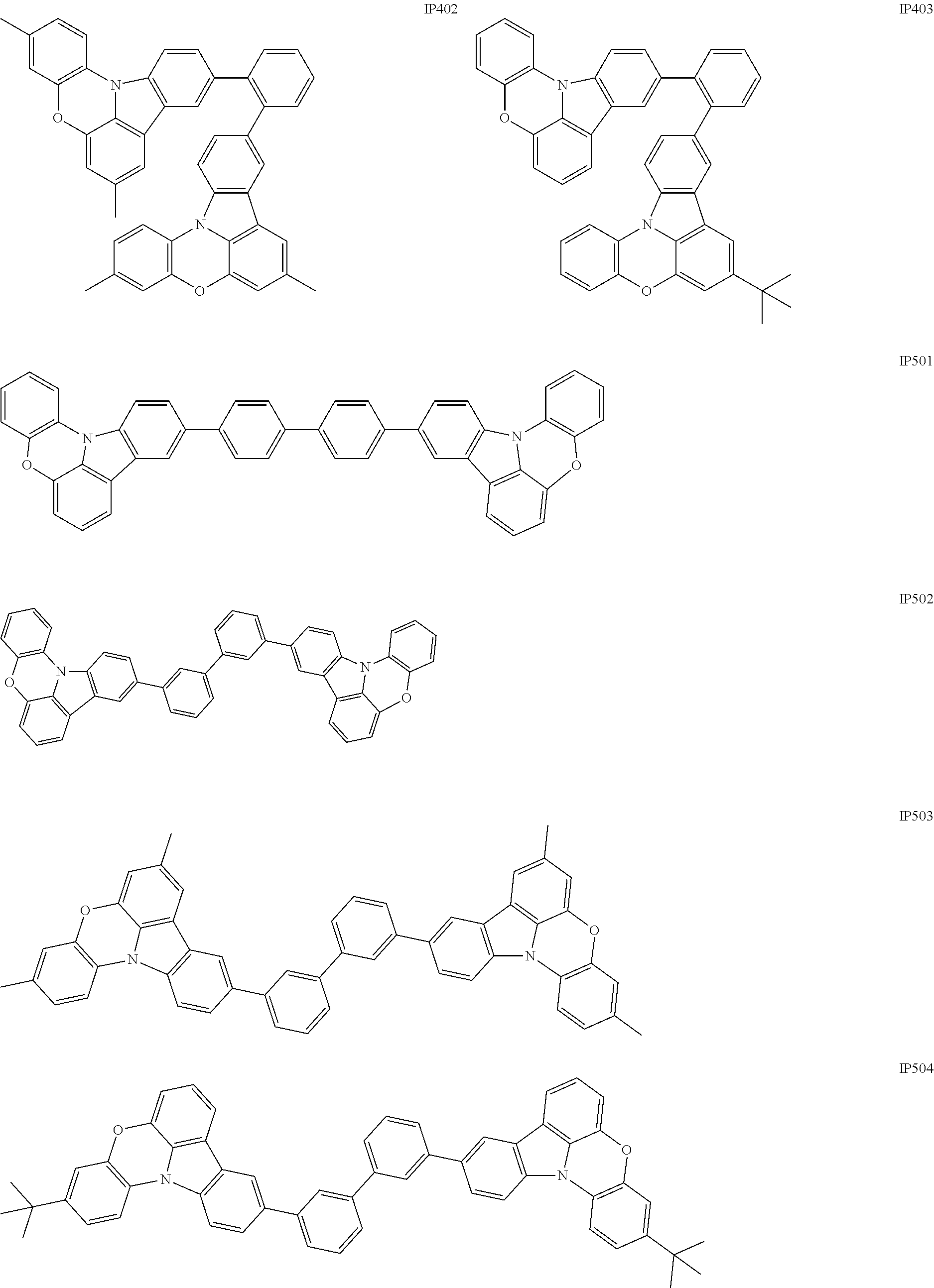

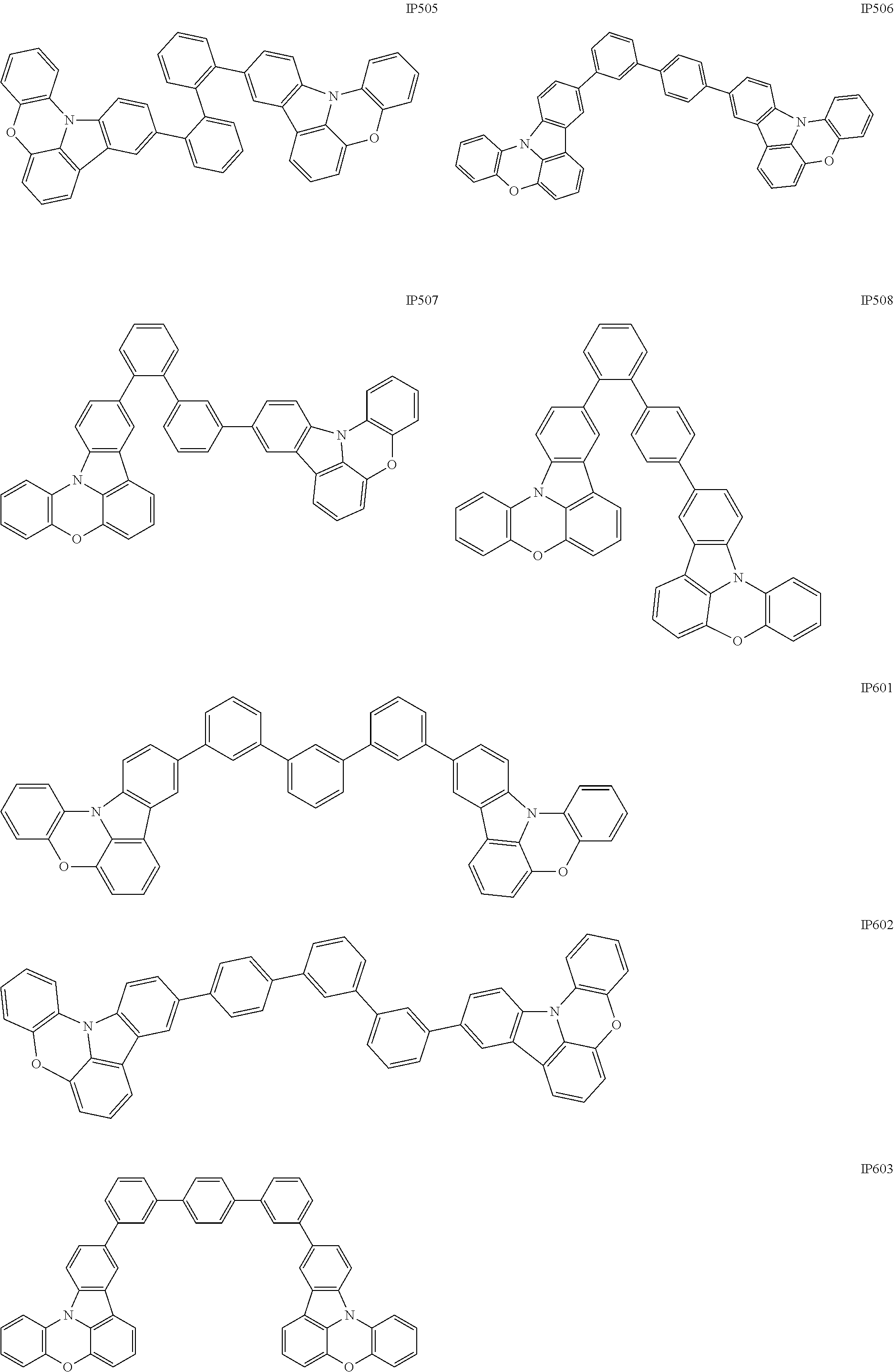

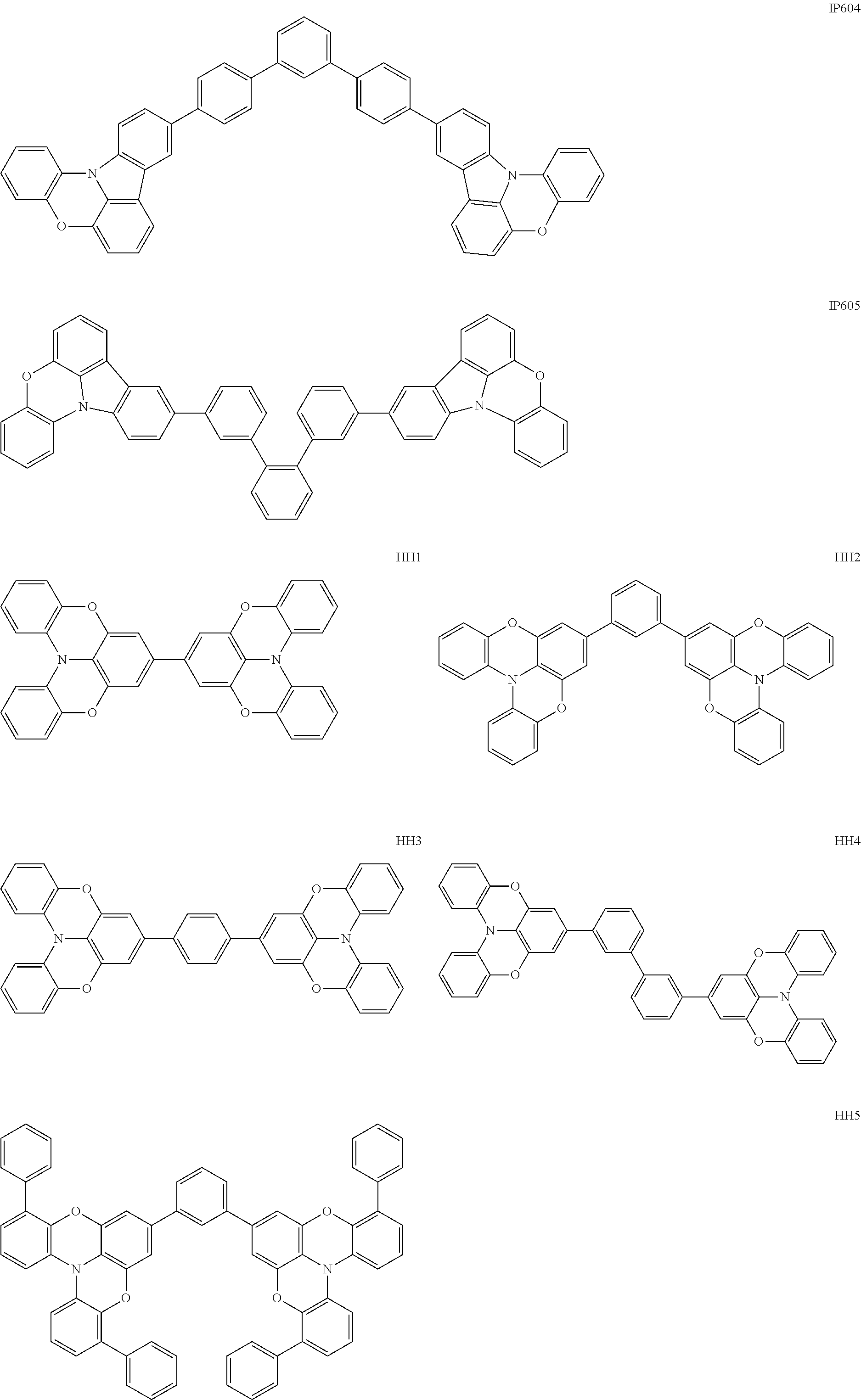

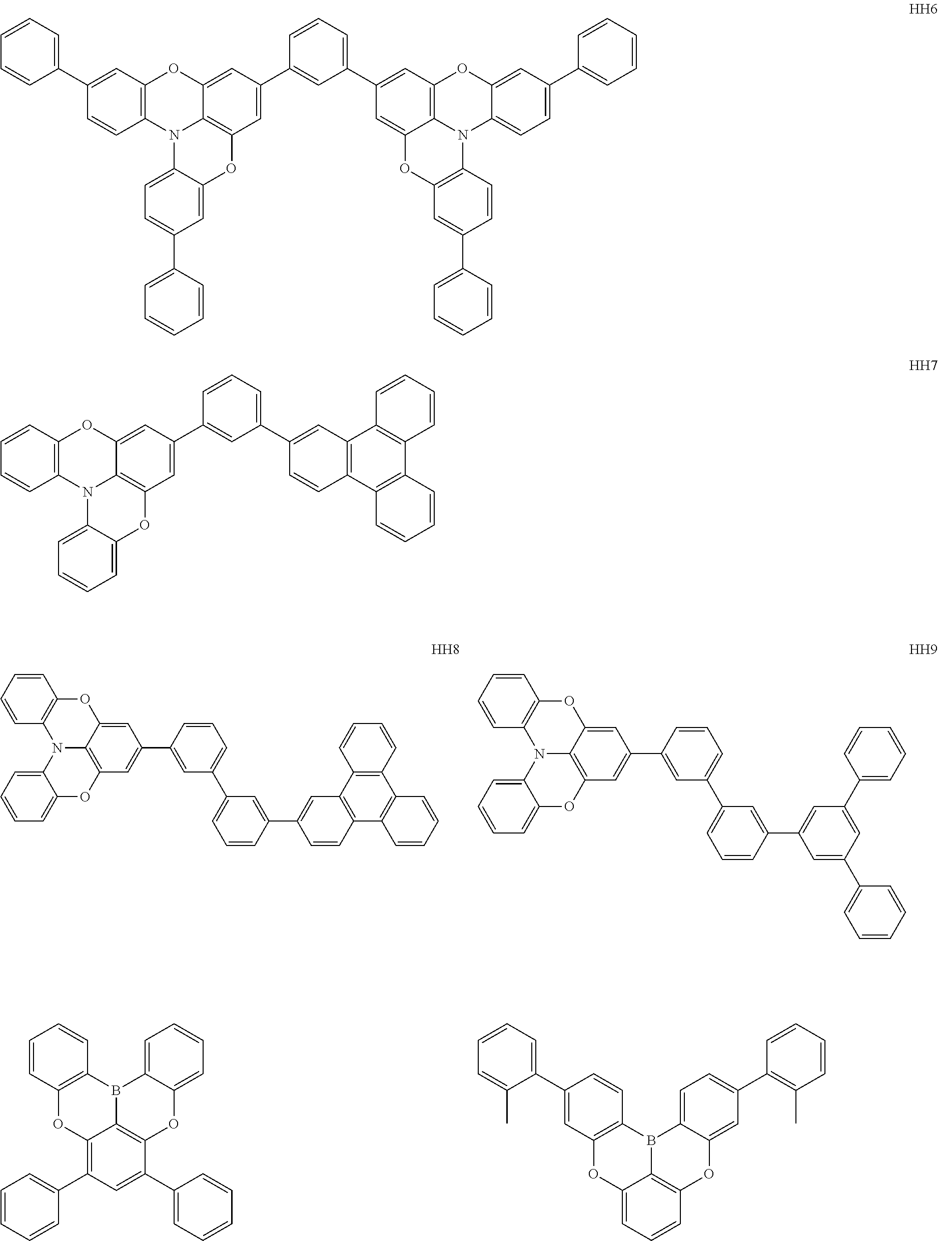

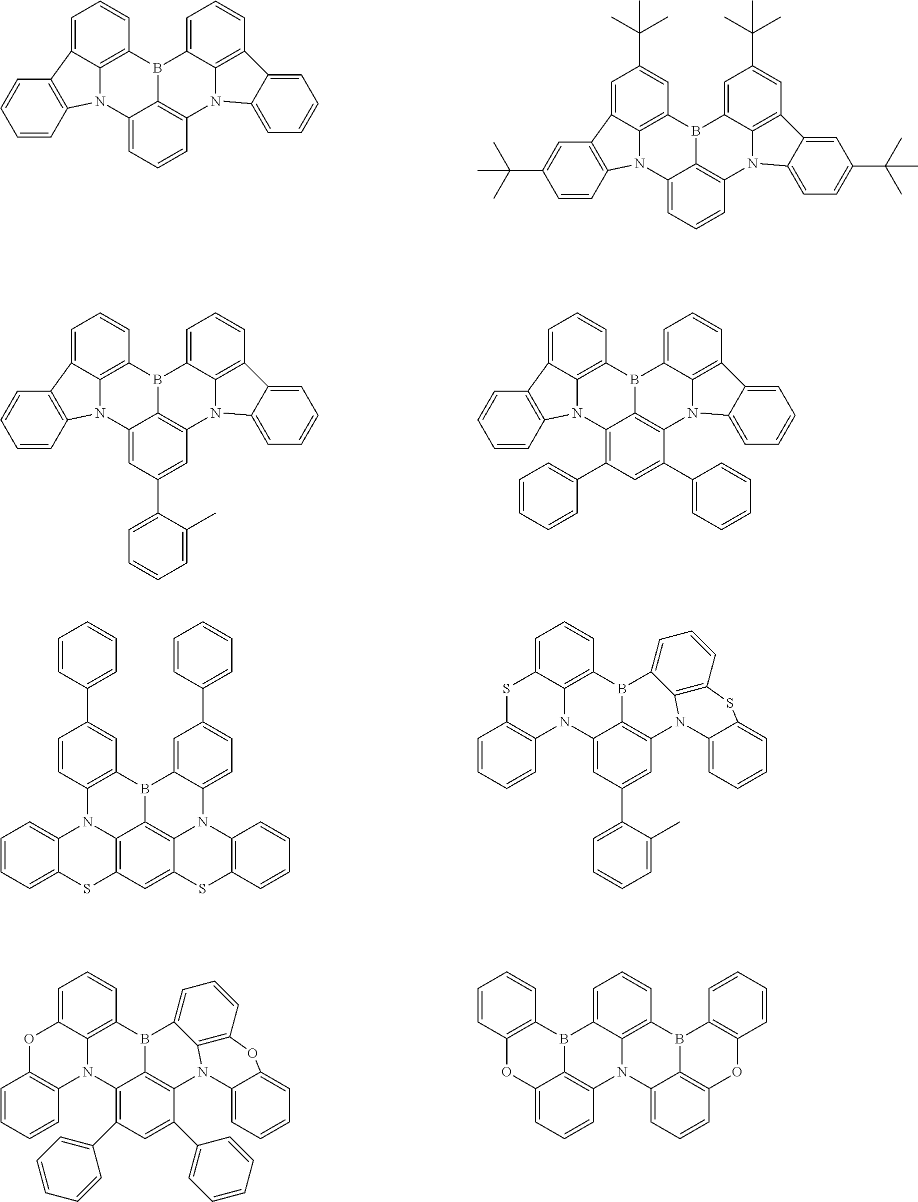

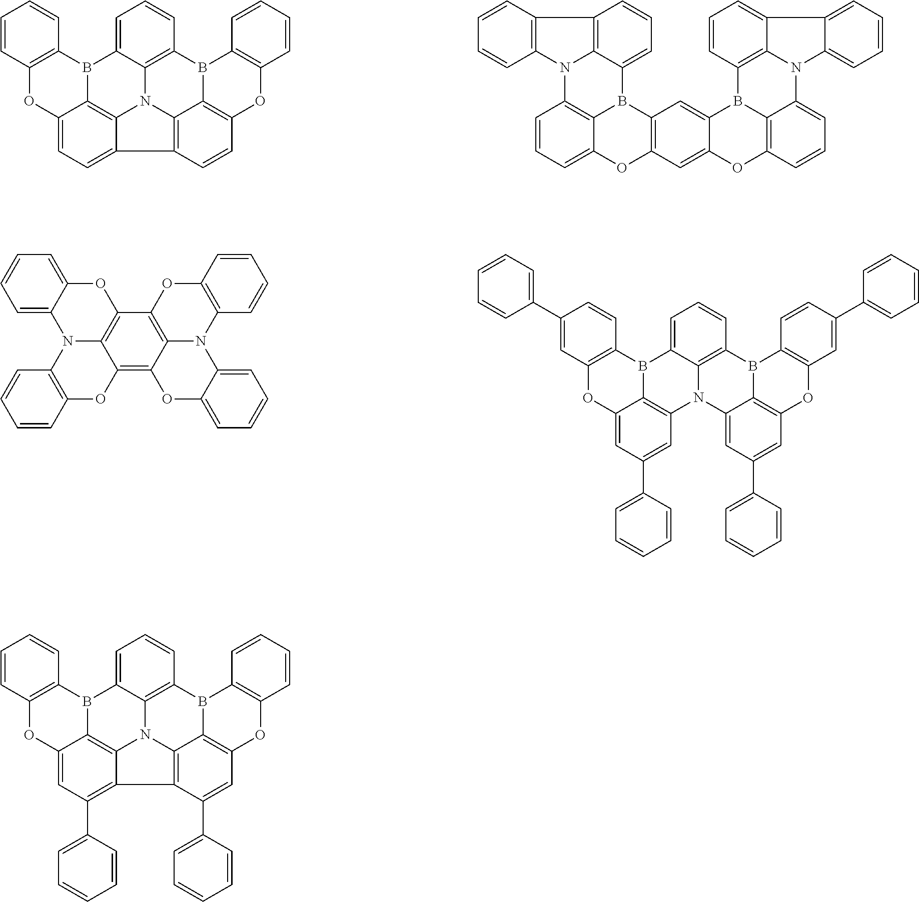

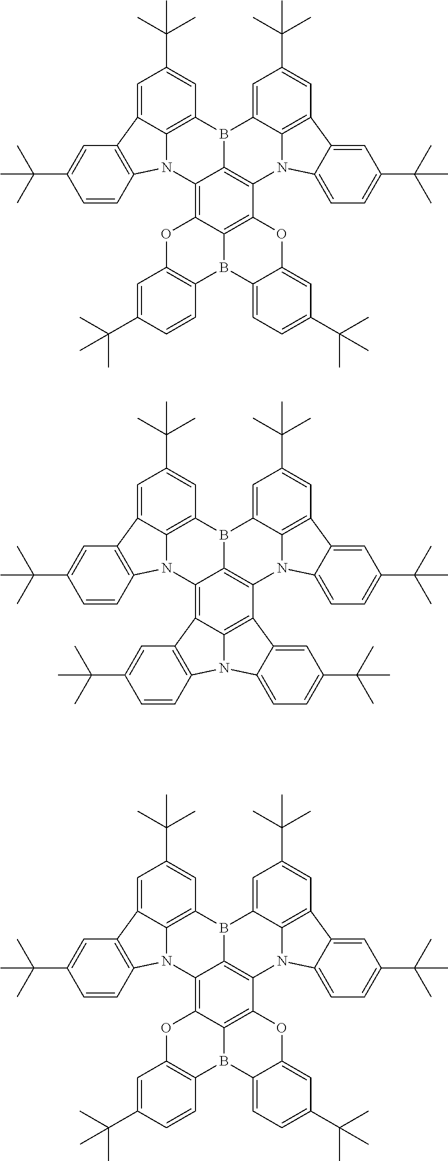

The first organic compound is also used as the third organic compound. In the present invention, when the first organic compound and the third organic compound are selected, it is sufficient that the relationships between LUMO and HOMO satisfy the above-described relationships [I] and [II].

### Second Organic Compound

The second organic compound is not particularly limited, except that freely rotatable single bonds are carbon-carbon bonds and at least one carbon atom of each carbon-carbon bond is an sp² carbon atom. Preferably, the second organic compound has a fluoranthene skeleton. Furthermore, the number of five-membered rings contained is preferably two or more. Specific examples of a fused-ring skeleton preferably contained in the second organic compound are illustrated below, but the fused-ring skeleton is not

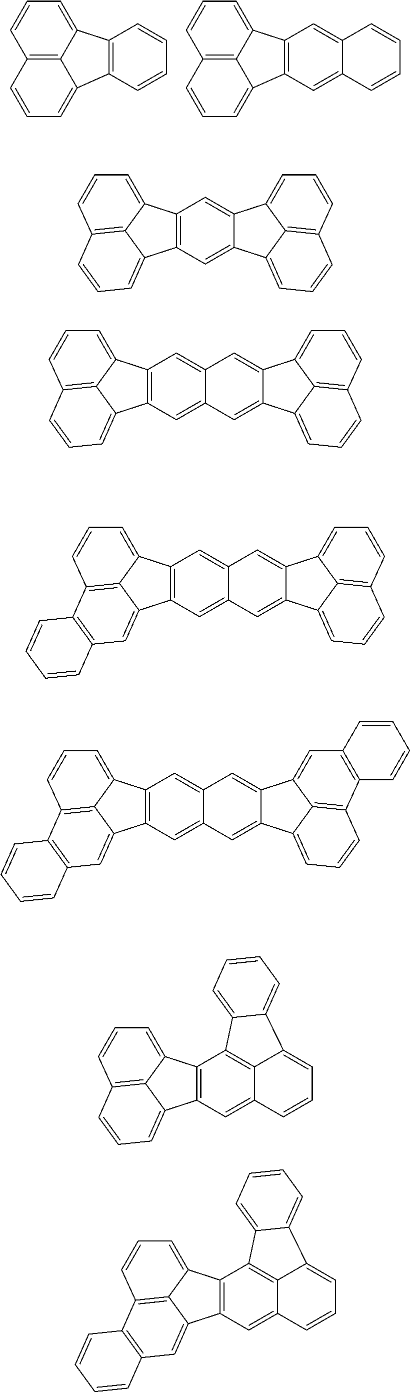

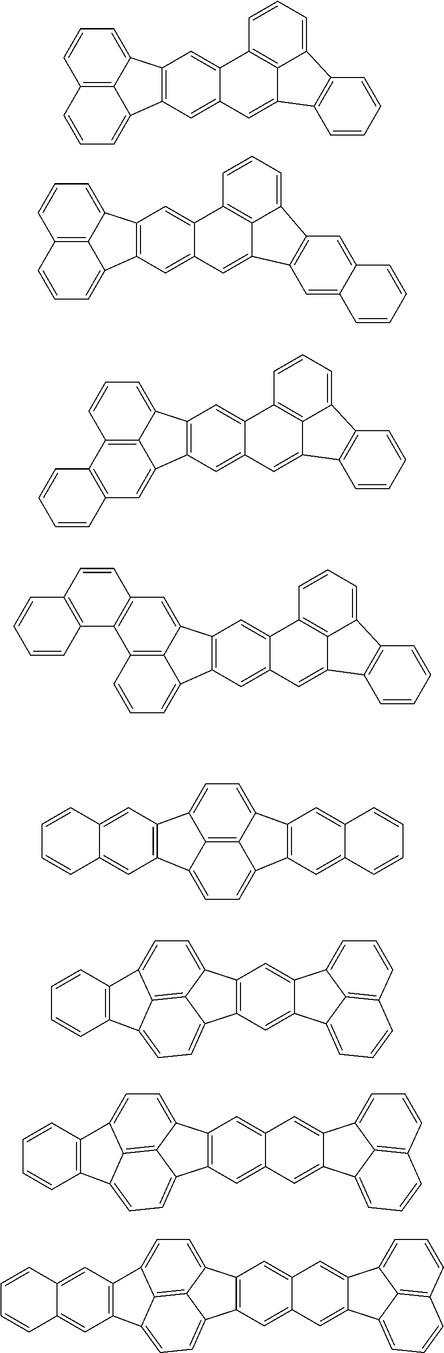

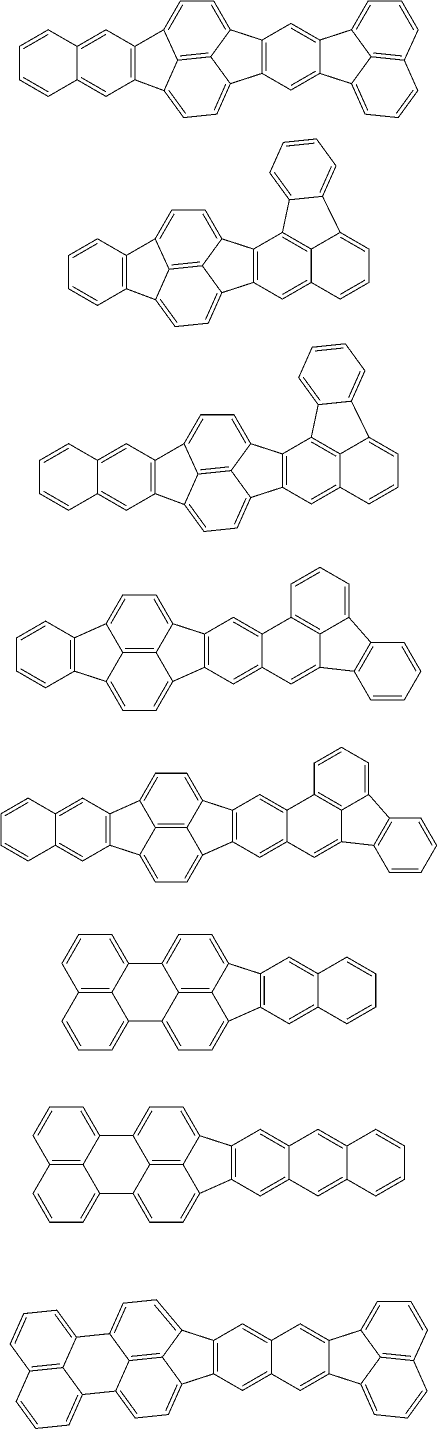

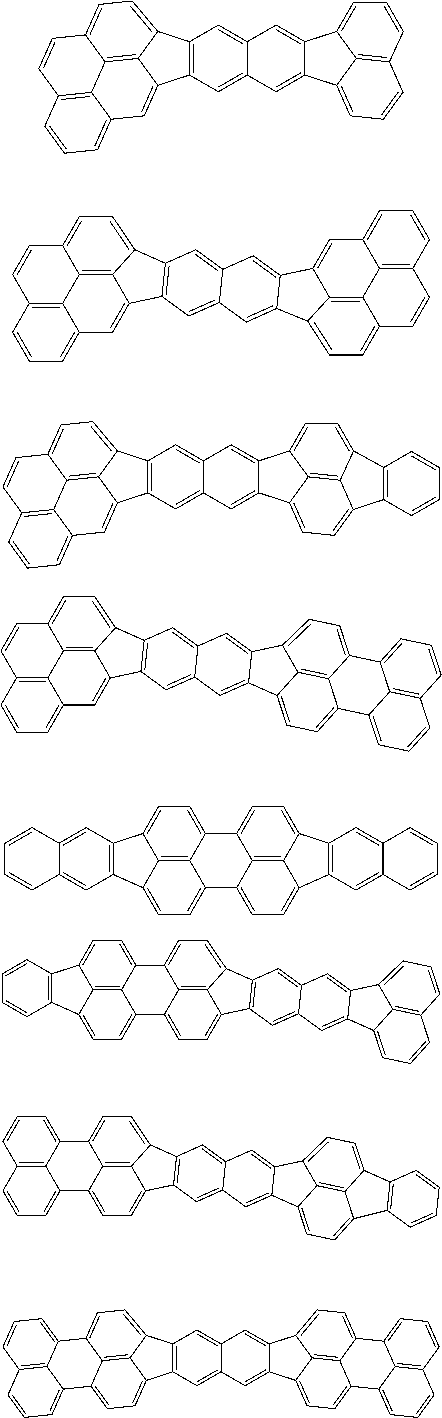

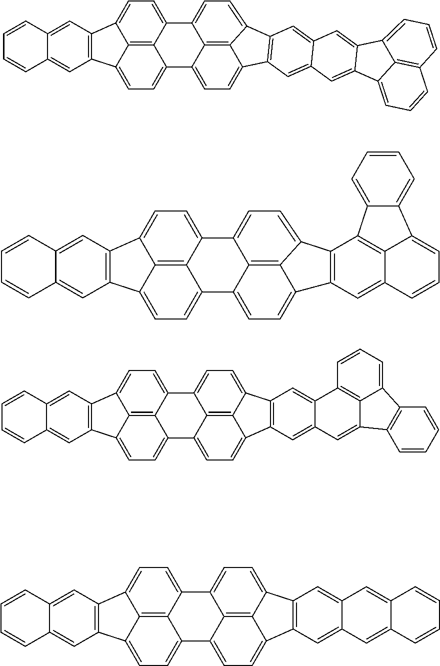

Preferred specific examples of the second organic compound are illustrated below, but the second organic compound is not limited thereto.

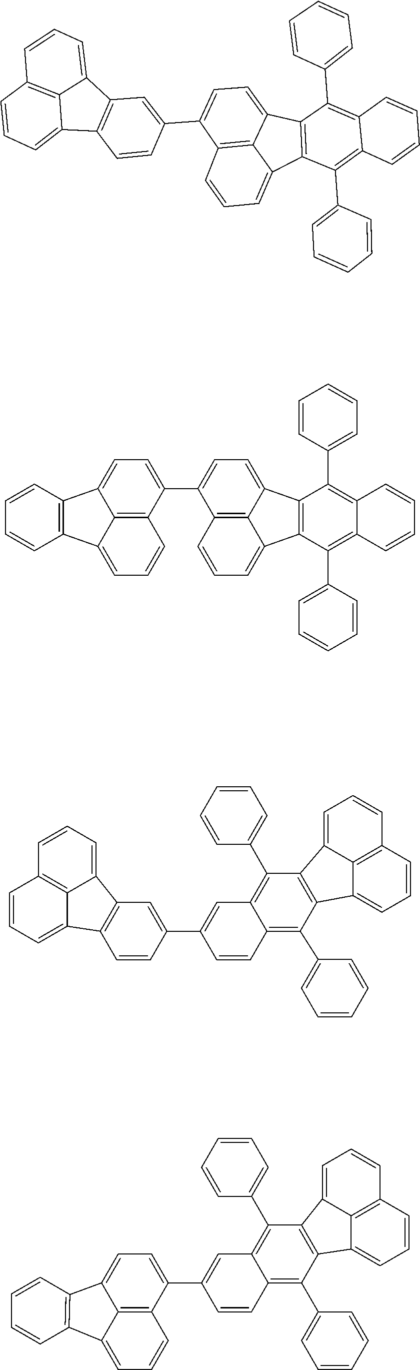

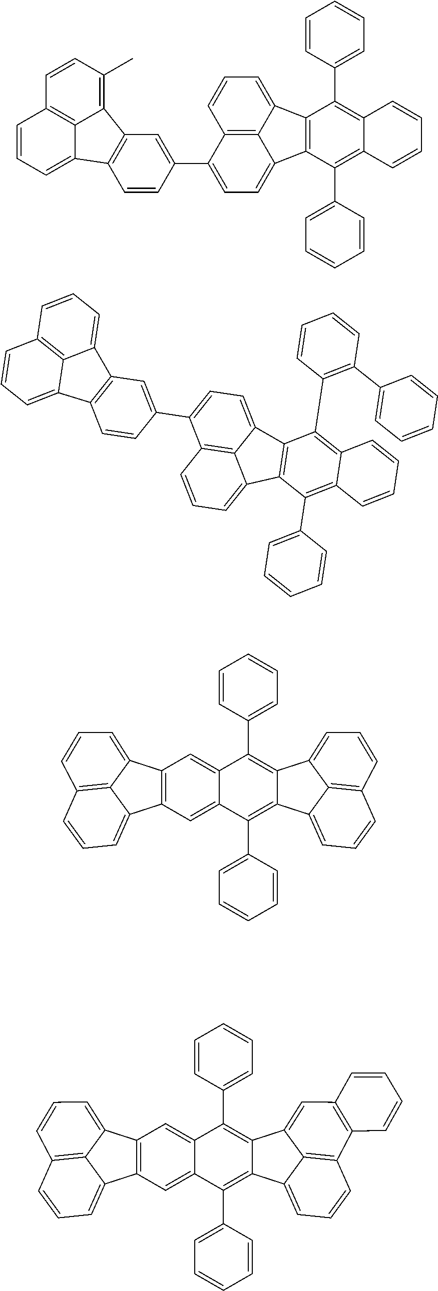

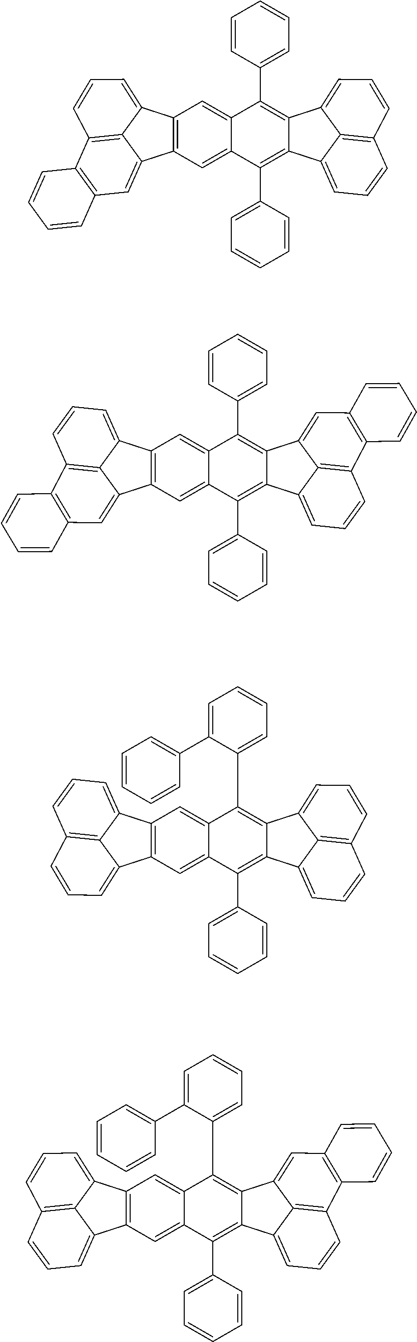

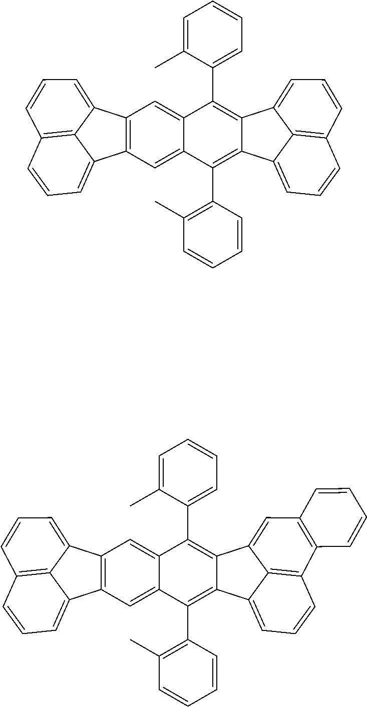

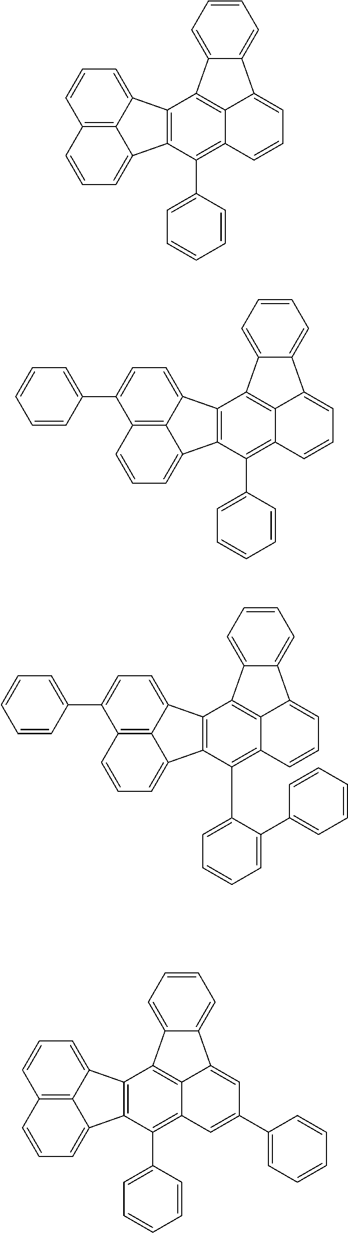

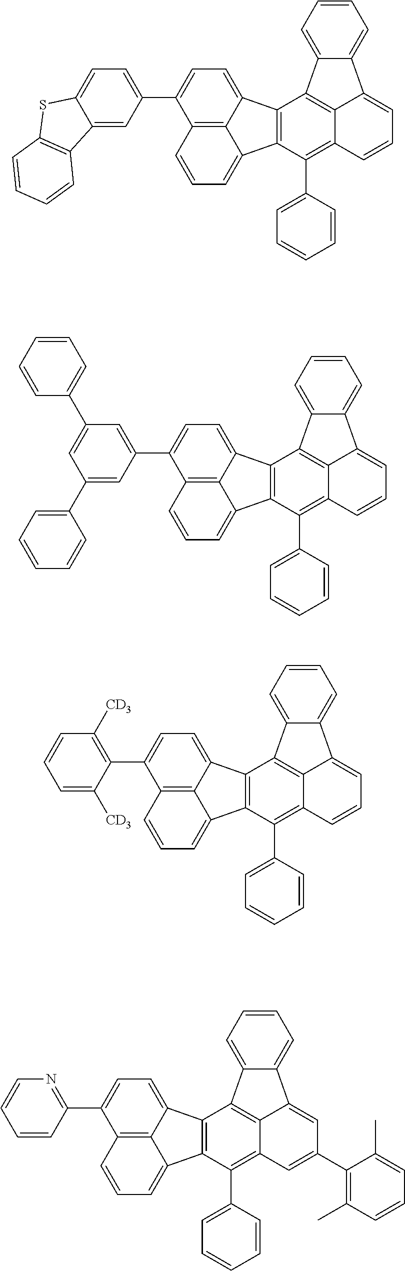

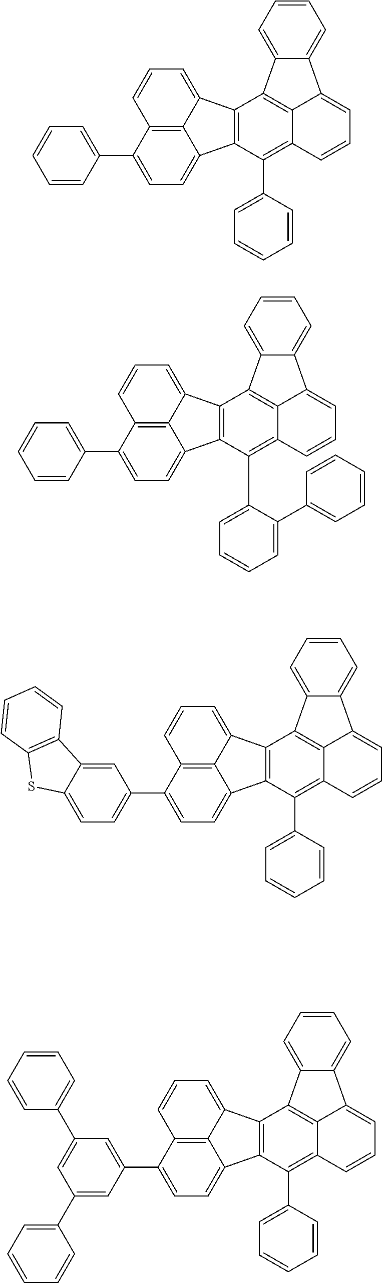

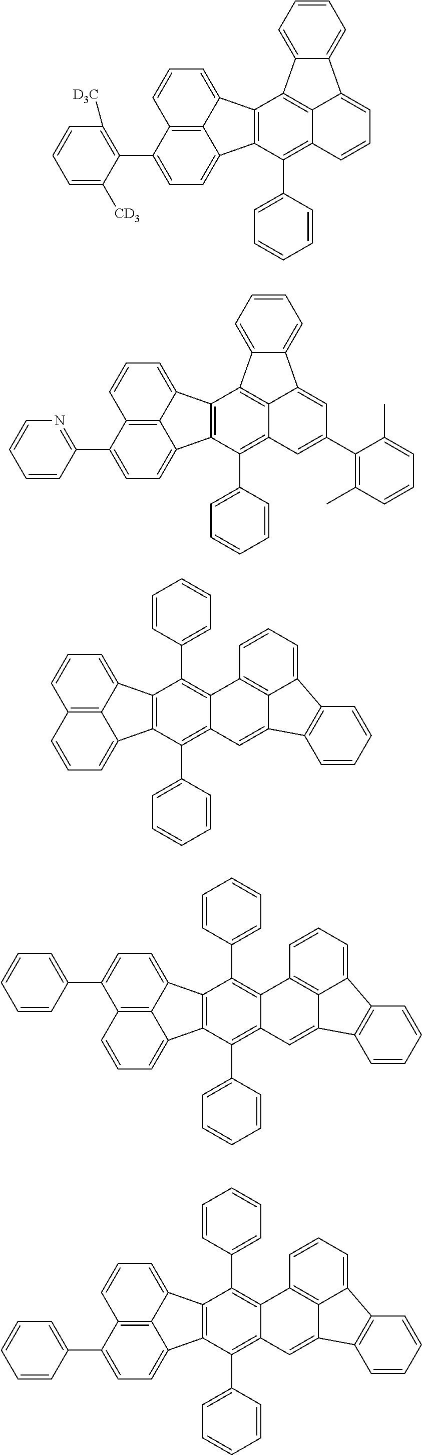

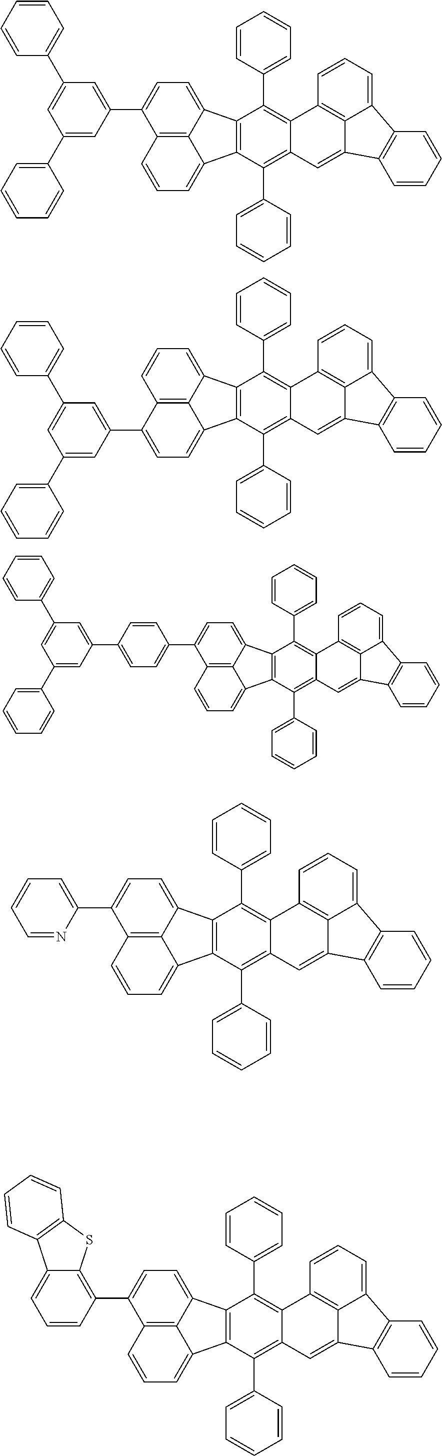

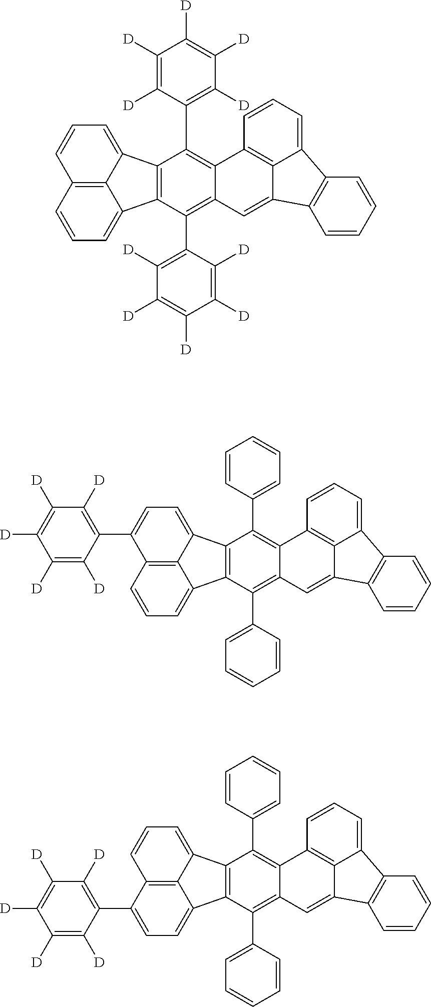 [SMILES: [2H]c1c([2H])c([2H])c(-c2c3cc4c5ccccc5c5cccc(c3c(-c3c([2H])c([2H])c([2H])c([2H])c3[2H])c3c6cccc7cccc(c23)c76)c54)c([2H])c1[2H] ; [2H]c1c([2H])c([2H])c(-c2ccc3c4c(-c5ccccc5)c5c(cc6c7ccccc7c7cccc5c76)c(-c5ccccc5)c4c4cccc2c43)c([2H])c1[2H] ; [2H]c1c([2H])c([2H])c(-c2ccc3c4c(-c5ccccc5)c5cc6c7ccccc7c7cccc(c5c(-c5ccccc5)c4c4cccc2c34)c76)c([2H])c1[2H]]

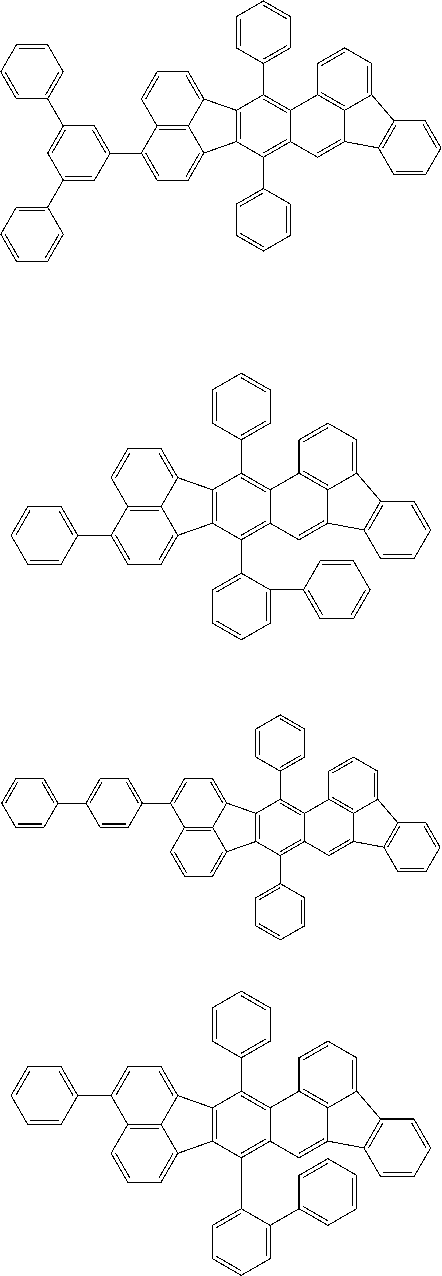

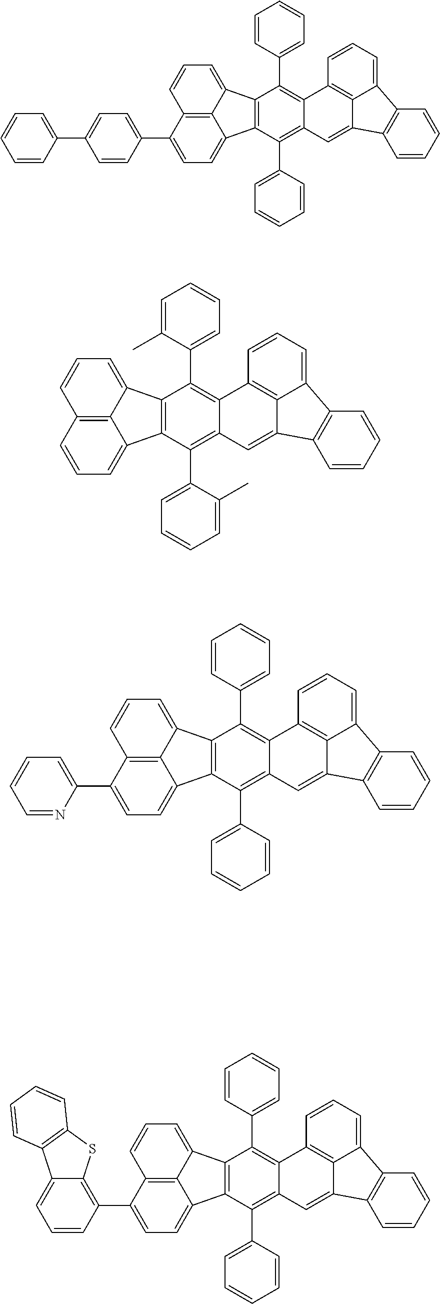

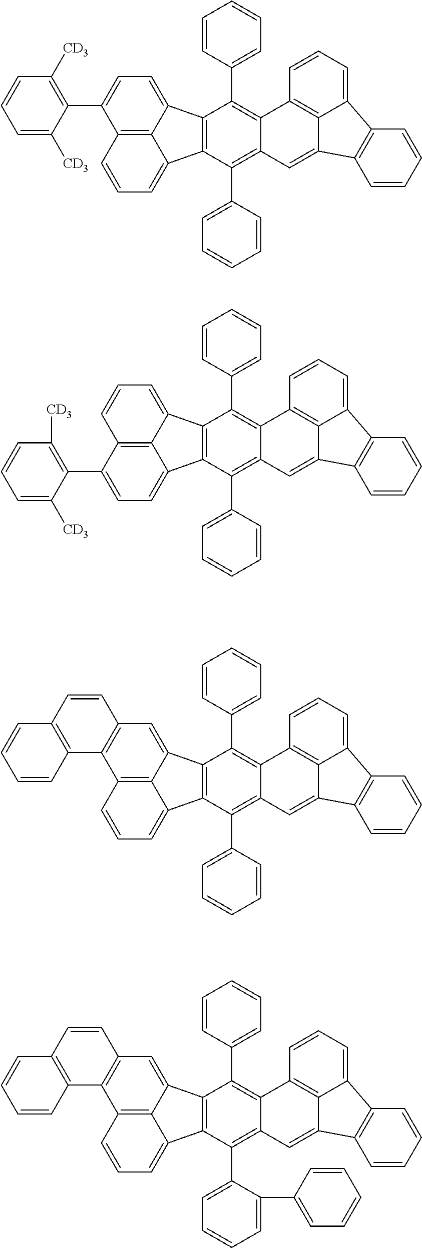

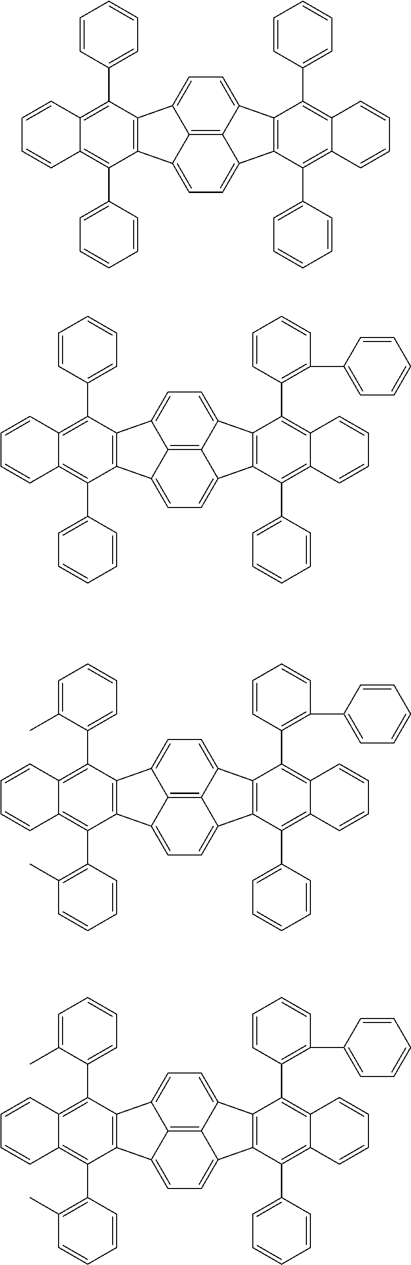

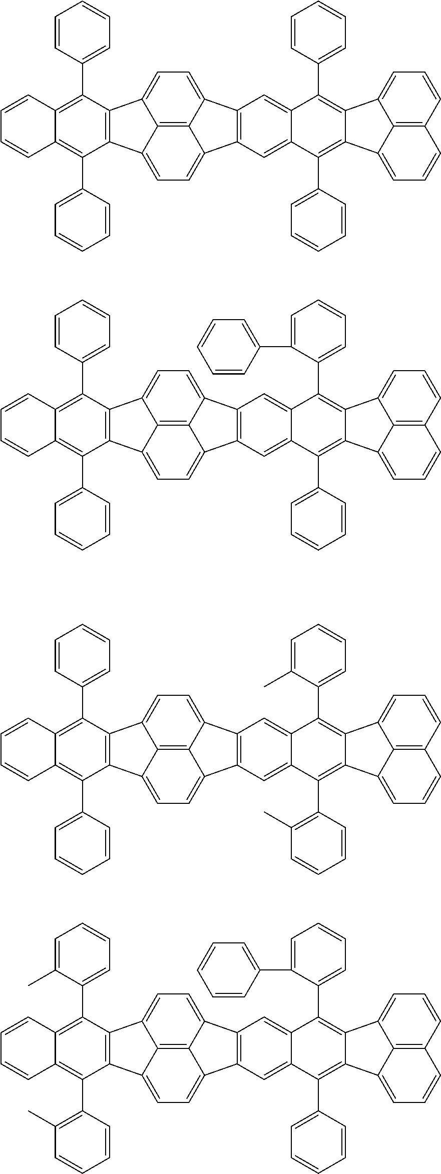

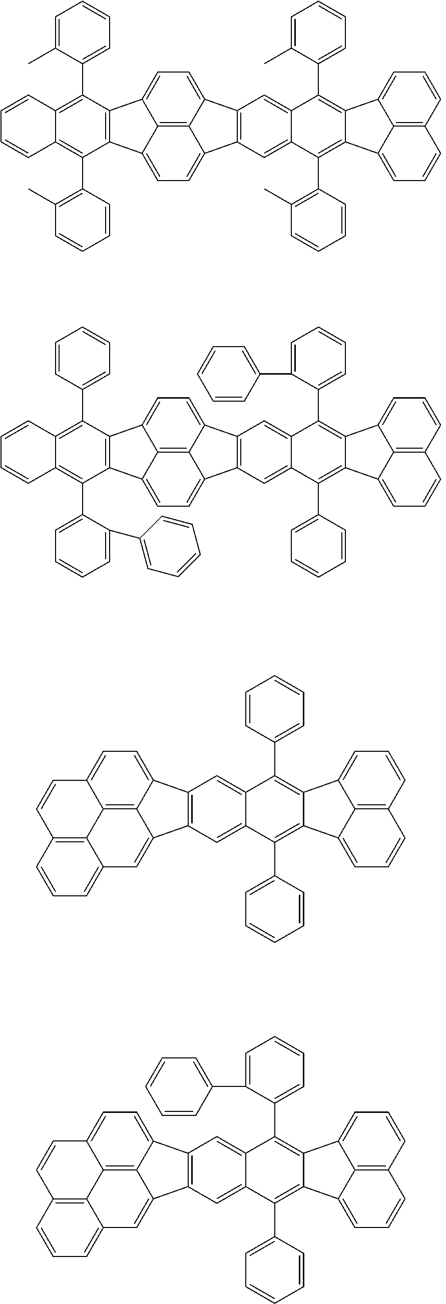

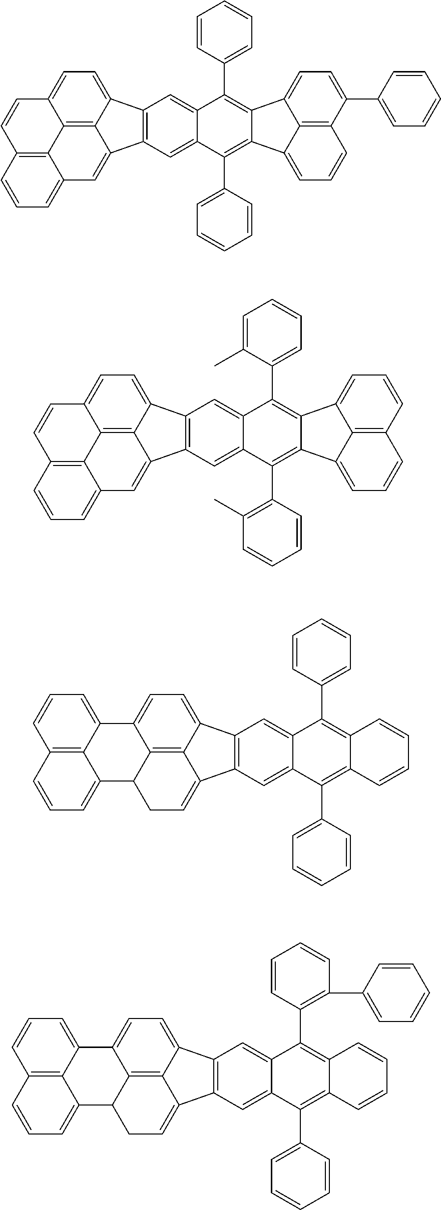

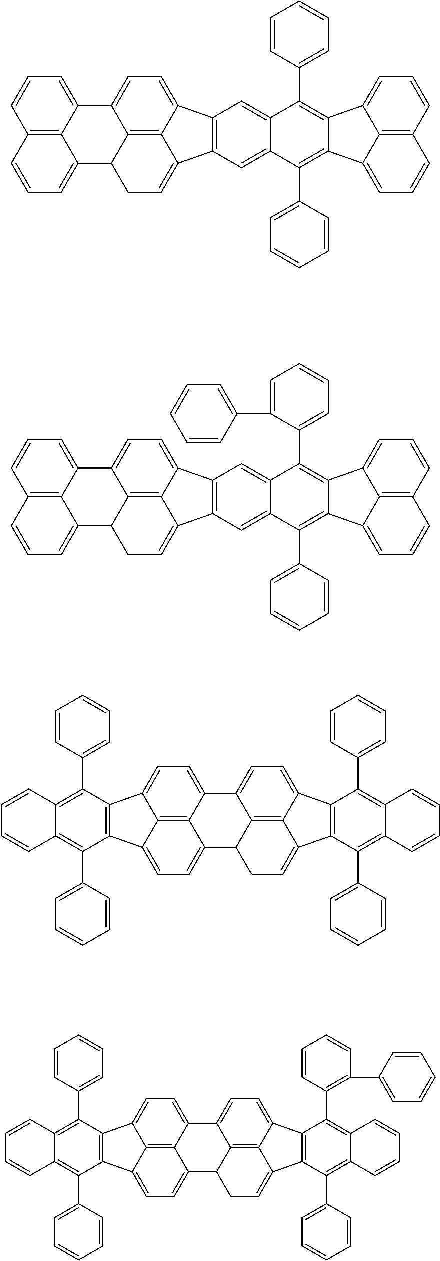

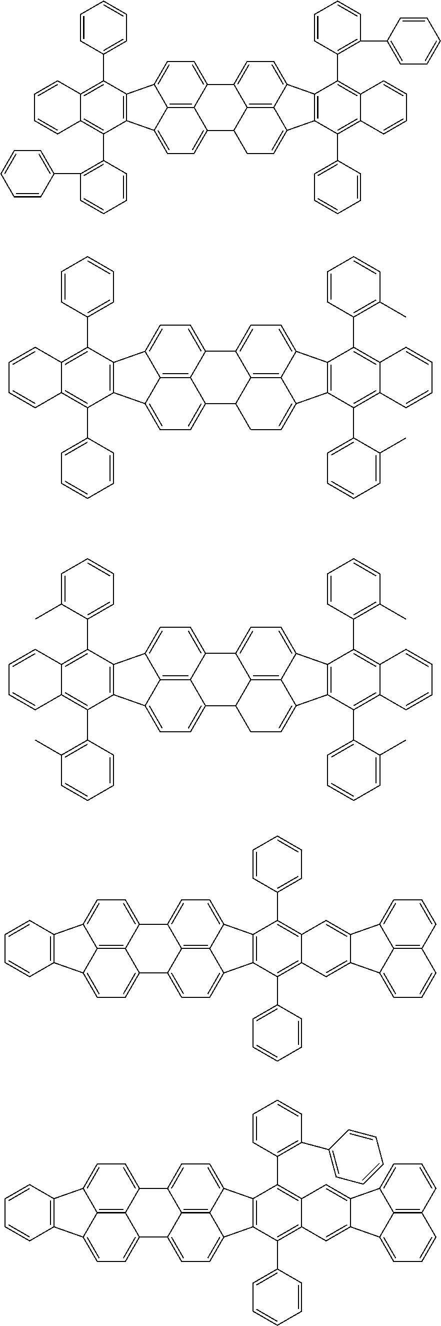

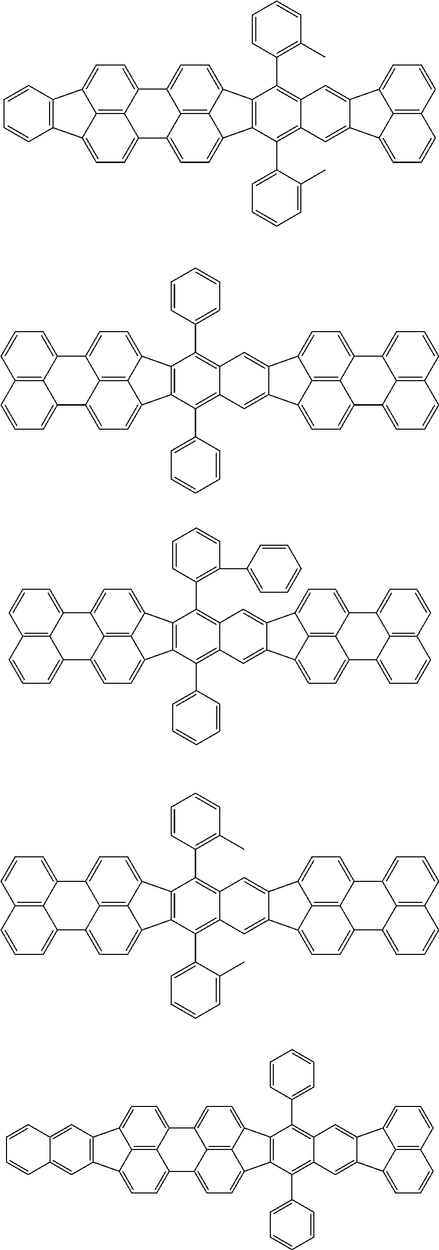

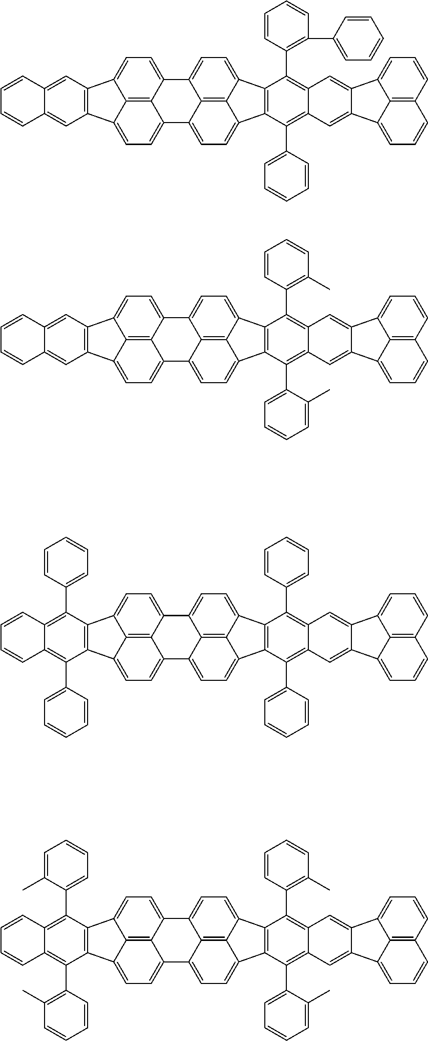

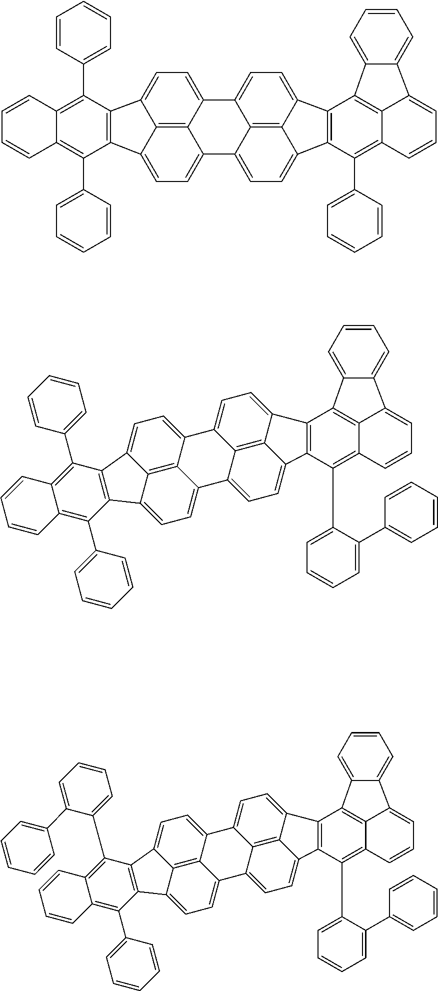

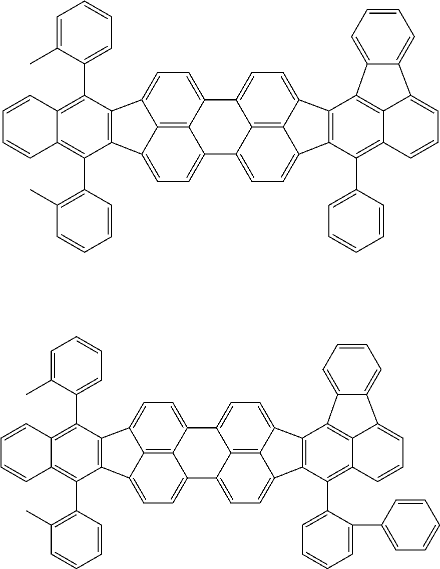

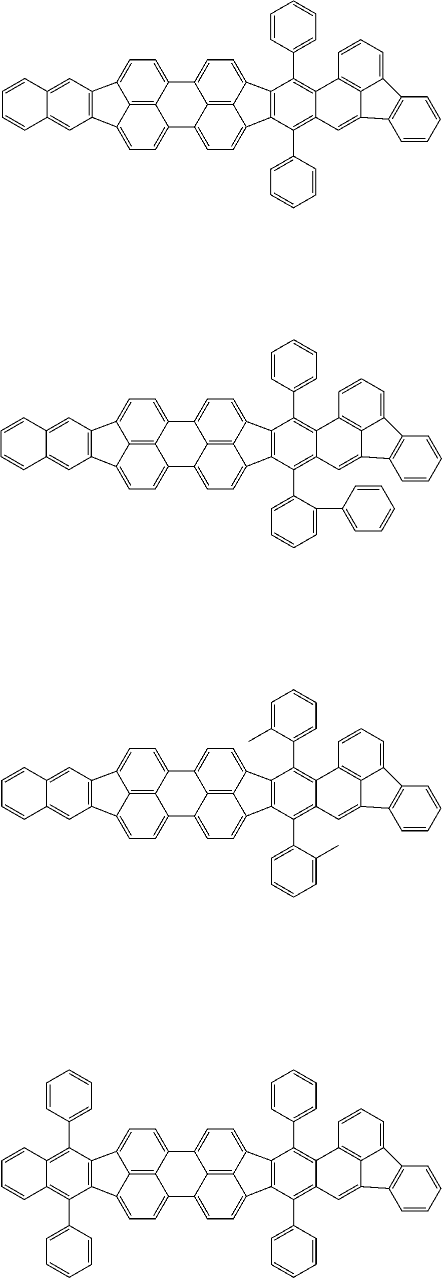

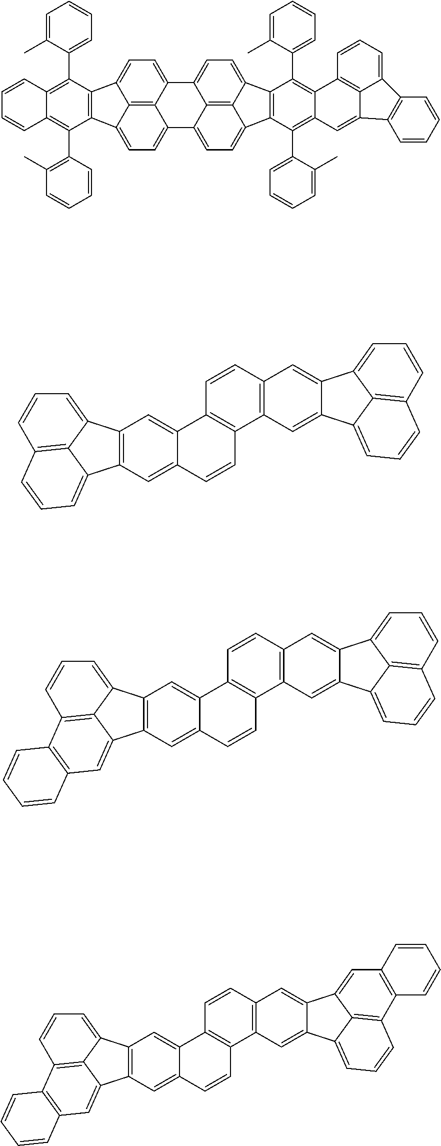

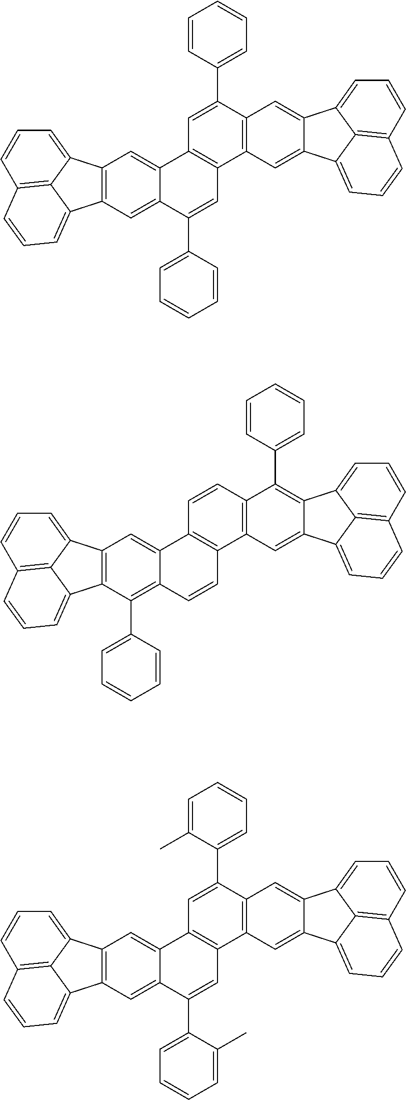

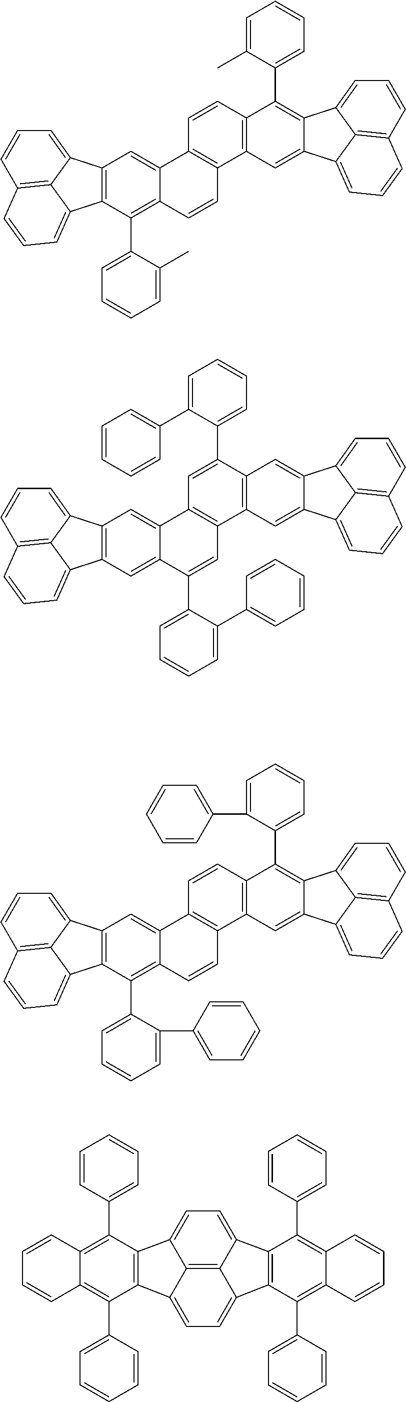

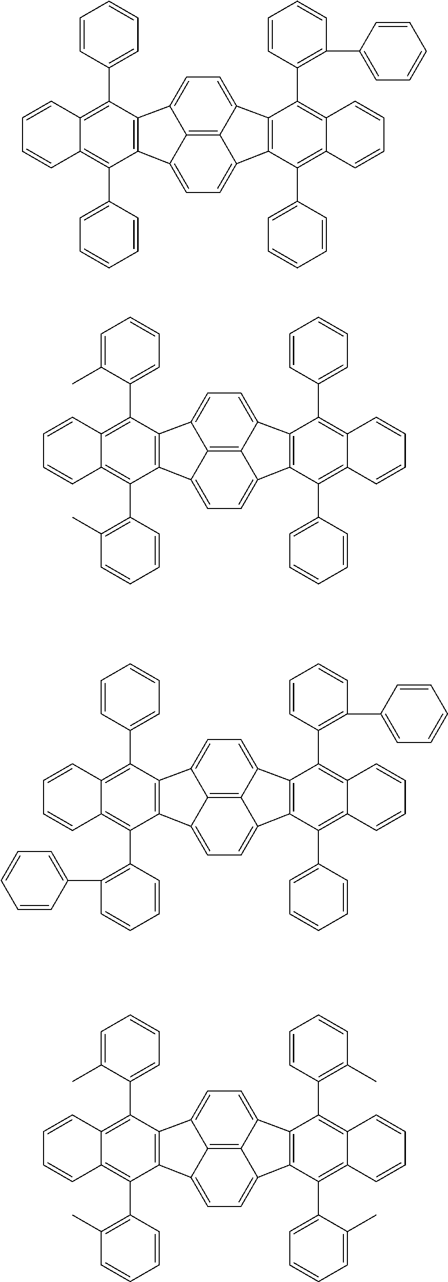

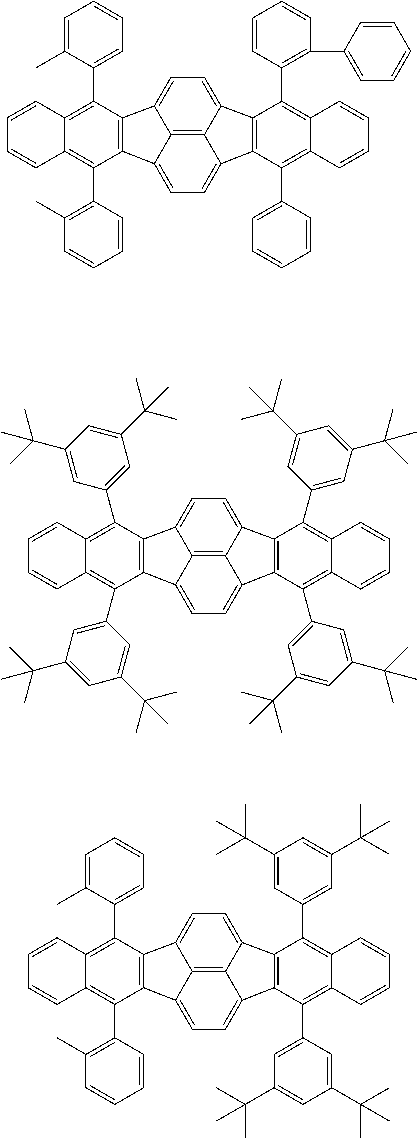

The organic light-emitting device of the present embodiment includes a pair of electrodes and an organic compound layer disposed between the pair of electrodes. The organic compound layer includes at least a light-emitting layer. When the organic compound layer is formed of a laminate including multiple layers, the organic compound layer may include, in addition to the light-emitting layer, a hole injection layer, a hole transport layer, an electron-blocking layer, a hole-exciton blocking layer, an electron transport layer, an electron injection layer, and so forth. The light-emitting layer may be formed of a single layer or a laminate including multiple layers.

In the organic light-emitting device of the present embodiment, at least one organic compound layer contains the first organic compound, the second organic compound, and the third organic compound. The layer containing the first to third organic compounds is preferably a light-emitting layer. In the case of the light-emitting layer, the third organic compound is a host, the second organic compound is a guest, and the first organic compound is an assist.

The term “host” used here refers to a compound having the highest proportion by mass in compounds contained in the light-emitting layer. The term “guest” refers to a compound that has a lower proportion by mass than the host in the compounds contained in the light-emitting layer and that is responsible for main light emission. The assist material is a compound that has a smaller proportion by mass than the host and a larger proportion by mass than the guest, among the compounds contained in the light-emitting layer. That is, the proportion by mass is host>assist≥guest.

The light-emitting layer according to the present embodiment contains at least three types. The concentration of the host is preferably 50% or more by mass and 99% or less by mass, more preferably 50% or more by mass and 98% or less by mass, based on the entire light-emitting layer. The concentration of the guest is preferably 0.1% or more by mass and 20% or less by mass based on the entire light-emitting layer. From the viewpoint of inhibiting concentration quenching, the concentration of the guest is preferably 1% or more by mass and 10% or less by mass. The concentration of the assist is preferably more than 0.5% by mass and 49.09% or less by mass, more preferably 2% or more by mass and 49.09% or less by mass, and even more preferably 5% or more by mass and 49.09% or less by mass, based on the entire light-emitting layer.

The guest may be uniformly contained in the entire layer in which the host serves as a matrix, or may be contained with a concentration gradient. The guest may be partially contained in a specific region in the layer, in other words, the light-emitting layer may have a region containing only the host without containing the guest.

The light-emitting layer may be a single layer or multiple layers. It is also possible to mix colors by containing a light-emitting material with another emission color. The term “multiple layer” refers to a state in which two or more light-emitting layers are stacked. In this case, the emission color of the organic light-emitting device is not particularly limited. More specifically, white or an intermediate color may be used. In the case of white, for example, when the emission color of a light-emitting layer is blue, another light-emitting layer emits light of a color different from blue, i.e., green or red. Furthermore, a third light-emitting layer that emits blue light and a charge generation layer may be disposed between the light-emitting layer or stacked light-emitting layer of the present invention and the first or second electrode. The charge generation layer functions as a tandem device. Electrons generated from the charge generation layer and holes injected from the first electrode recombine to generate excitons. Holes generated from the charge generation layer and electrons injected from the second electrode recombine to generate excitons. Thus, the internal quantum efficiency is doubled. In this case, the organic light-emitting device of the present invention can be used as a blue light-emitting layer, serving as the complementary color to yellow light emission, on one side of the tandem device. Accordingly, by using the blue light-emitting layer formed of the light-emitting layer of the present invention in combination with a yellow light-emitting layer in a tandem device configuration, it is possible to provide a white light-emitting device. Film formation is also performed by vapor deposition or coating.

The specific device configuration of the organic light-emitting device of the present embodiment includes multilayer device configurations described in (1) to (6) below, in which electrode layers and organic compound layers are sequentially stacked on a substrate. In any device configuration, the organic compound layer always includes a light-emitting layer containing a light-emitting material.
(1) Anode/light-emitting layer/cathode
(2) Anode/hole transport layer/light-emitting layer/electron transport layer/cathode
(3) Anode/hole transport layer/light-emitting layer/electron transport layer/electron injection layer/cathode
(4) Anode/hole injection layer/hole transport layer/light-emitting layer/electron transport layer/cathode
(5) Anode/hole injection layer/hole transport layer/light-emitting layer/electron transport layer/electron injection layer/cathode
(6) Anode/hole transport layer/electron-blocking layer/light-emitting layer/hole-blocking layer/electron transport layer/cathode

However, these device configuration examples are only very basic device configurations, and the device configuration is not limited thereto. It is possible to use various layer configurations: For example, an insulating layer, an adhesive layer, or an interference layer is disposed at the interface between the electrode and the organic compound layer. The electron transport layer or the hole transport layer is formed of two layers having different ionization potentials. Furthermore, the light-emitting layer is formed of two layers containing different light-emitting materials.

Of the device configurations described in (1) to (6) above, configuration (6) is preferred because it has both the electron-blocking layer and the hole-blocking layer. That is, in the case of (6) including the electron-blocking layer and the hole-blocking layer, both carriers, holes and electrons, can be reliably confined within the light-emitting layer, resulting in an organic light-emitting device with no carrier leakage and high luminous efficiency.

In the organic light-emitting device of the present invention, all of the freely rotatable single bonds in the first to third organic compounds are carbon-carbon bonds. Furthermore, in the second organic compound, at least one carbon atom of each carbon-carbon bond is an sp² carbon atom. The light-emitting layer containing the first to third organic compounds has a stable molecular structure and thus has good durability characteristics. Therefore, it is preferable that the compounds of the electron-blocking layer and the hole-blocking layer in contact with the light-emitting layer also have stable structures. For example, the hole-blocking layer needs to be stable against holes. Thus, the compound of the hole-blocking layer is preferably an organic compound with low reactivity, more preferably an organic compound composed only of a hydrocarbon. For example, the electron-blocking layer also needs to be stable against electrons, and thus is preferably composed of an organic compound with low reactivity, more preferably an organic compound in which all of the freely rotatable single bonds are carbon-carbon bonds, preferably sp² carbon-sp² carbon bonds.

A mode of extracting light output from a light-emitting layer (device configuration) may be what is called a bottom emission mode in which light is extracted from an electrode on the substrate side, or what is called a top emission mode in which light is extracted from the side opposite to the substrate. In addition, a double-sided extraction mode in which light is extracted from the substrate side and the side opposite to the substrate can also be used.

### Other Compounds

The first to third organic compounds according to the present invention can be used as constituent materials for organic compound layers other than the light-emitting layer included in the organic light-emitting device of the present embodiment. Specifically, the compounds may be used as constituent materials for an electron transport layer, an electron injection layer, a hole transport layer, a hole injection layer, a hole-blocking layer, and so forth. In this case, the emission color of the organic light-emitting device is not particularly limited. More specifically, white or an intermediate color may be used.

In the organic light-emitting device according to the present embodiment, for example, a conventionally known low-molecular-weight or high-molecular-weight hole injection compound, hole transport compound, compound serving as a host, light-emitting compound, electron injection compound, or electron transport compound can be used together as needed. Examples of these compounds will be described below.

A hole injection-transport material is preferably a material having a high hole mobility so as to facilitate the injection of holes from the anode and to transport the injected holes to the light-emitting layer. To inhibit a deterioration in film quality, such as crystallization, in the organic light-emitting device, a material having a high glass transition temperature is preferred. Examples of low- or high-molecular-weight materials having the ability to inject and transport holes include triarylamine derivatives, aryl carbazole derivatives, phenylenediamine derivatives, stilbene derivatives, phthalocyanine derivatives, porphyrin derivatives, poly(vinyl carbazole), polythiophene, and other conductive polymers. Furthermore, the above-described hole injection-transport material is also suitable for use in an electron-blocking layer. The following are specific examples of compounds used as the hole injection-transport materials, but the hole injection-transport materials are not limited thereto.

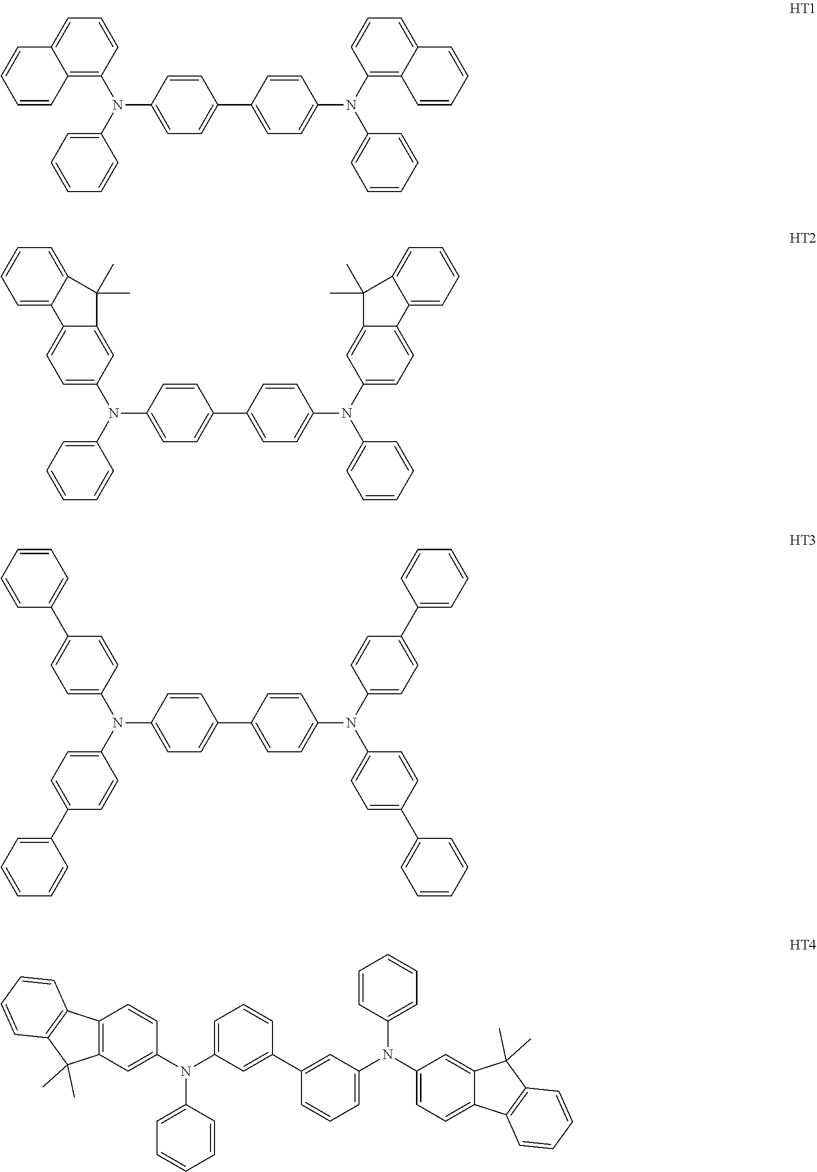 [SMILES: c1ccc(N(c2ccc(-c3ccc(N(c4ccccc4)c4cccc5ccccc45)cc3)cc2)c2cccc3ccccc23)cc1 ; CC1(C)c2ccccc2-c2ccc(N(c3ccccc3)c3ccc(-c4ccc(N(c5ccccc5)c5ccc6c(c5)C(C)(C)c5ccccc5-6)cc4)cc3)cc21 ; c1ccc(-c2ccc(N(c3ccc(-c4ccccc4)cc3)c3ccc(-c4ccc(N(c5ccc(-c6ccccc6)cc5)c5ccc(-c6ccccc6)cc5)cc4)cc3)cc2)cc1 ; CC1(C)c2ccccc2-c2ccc(N(c3ccccc3)c3cccc(-c4cccc(N(c5ccccc5)c5ccc6c(c5)C(C)(C)c5ccccc5-6)c4)c3)cc21]

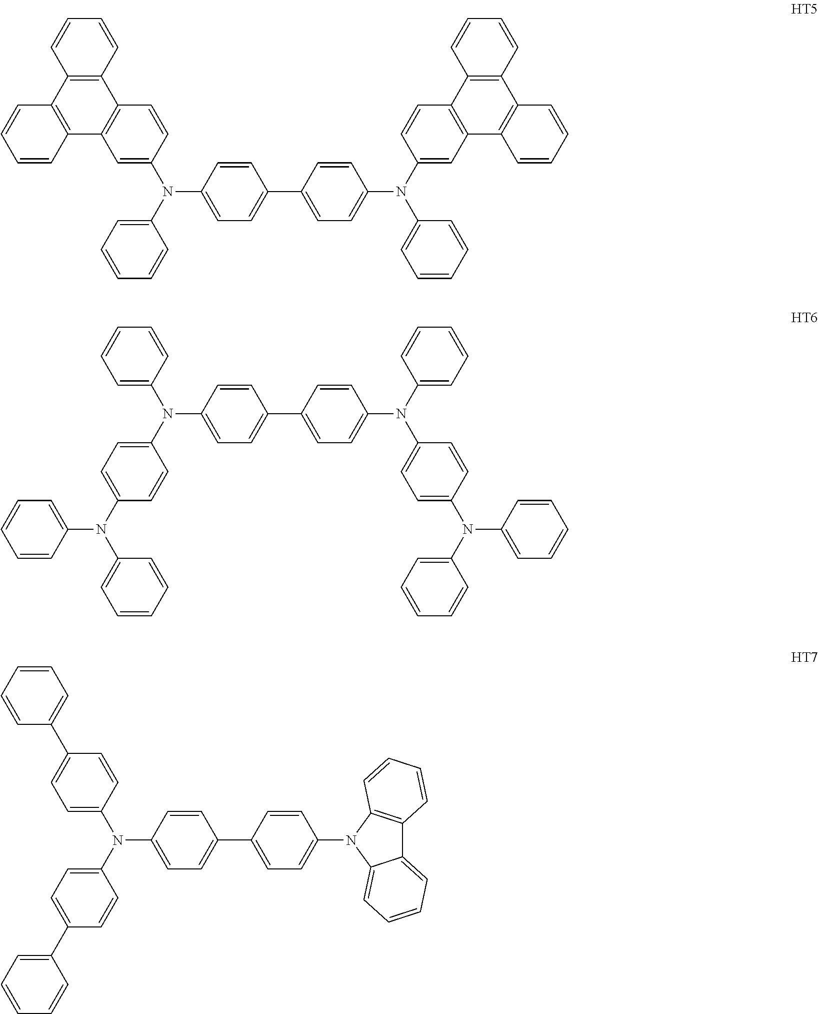 [SMILES: c1ccc(N(c2ccc(-c3ccc(N(c4ccccc4)c4ccc5c6ccccc6c6ccccc6c5c4)cc3)cc2)c2ccc3c4ccccc4c4ccccc4c3c2)cc1 ; c1ccc(N(c2ccccc2)c2ccc(N(c3ccccc3)c3ccc(-c4ccc(N(c5ccccc5)c5ccc(N(c6ccccc6)c6ccccc6)cc5)cc4)cc3)cc2)cc1 ; c1ccc(-c2ccc(N(c3ccc(-c4ccccc4)cc3)c3ccc(-c4ccc(-n5c6ccccc6c6ccccc65)cc4)cc3)cc2)cc1]

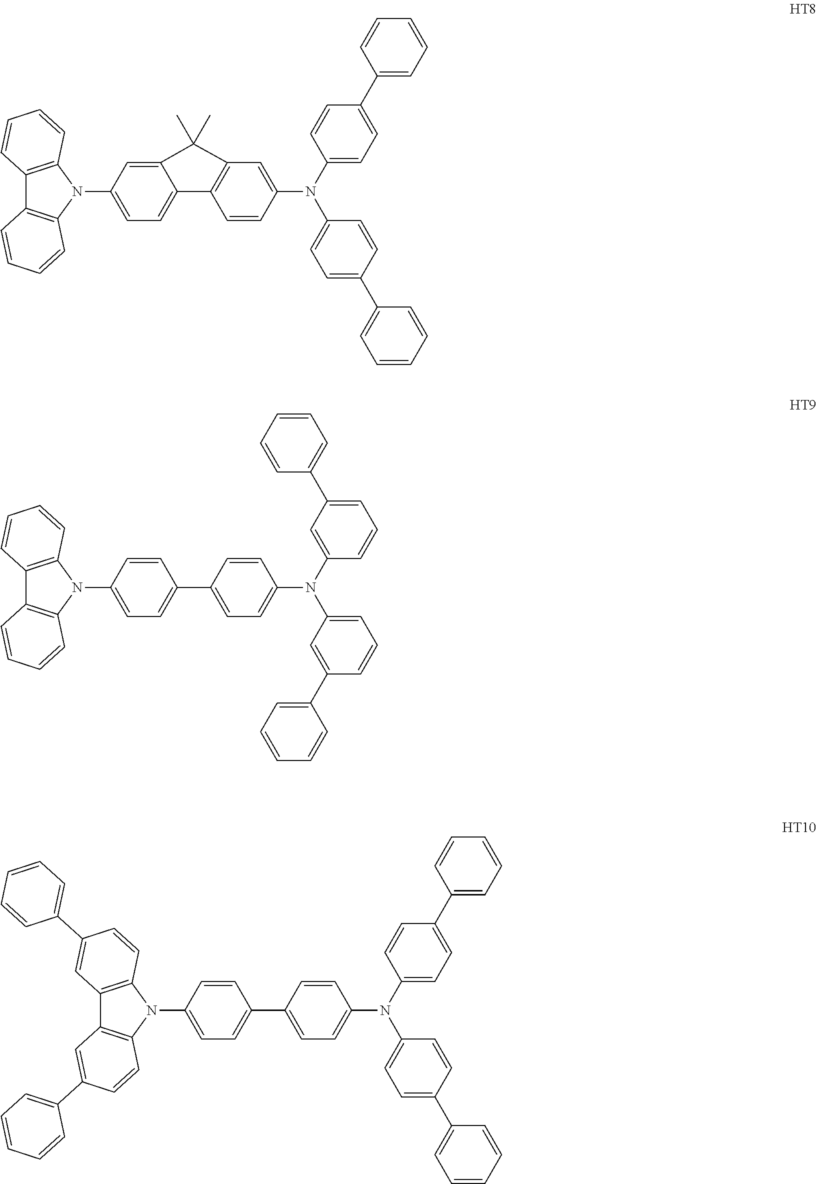 [SMILES: CC1(C)c2cc(N(c3ccc(-c4ccccc4)cc3)c3ccc(-c4ccccc4)cc3)ccc2-c2ccc(-n3c4ccccc4c4ccccc43)cc21 ; c1ccc(-c2cccc(N(c3ccc(-c4ccc(-n5c6ccccc6c6ccccc65)cc4)cc3)c3cccc(-c4ccccc4)c3)c2)cc1 ; c1ccc(-c2ccc(N(c3ccc(-c4ccccc4)cc3)c3ccc(-c4ccc(-n5c6ccc(-c7ccccc7)cc6c6cc(-c7ccccc7)ccc65)cc4)cc3)cc2)cc1]

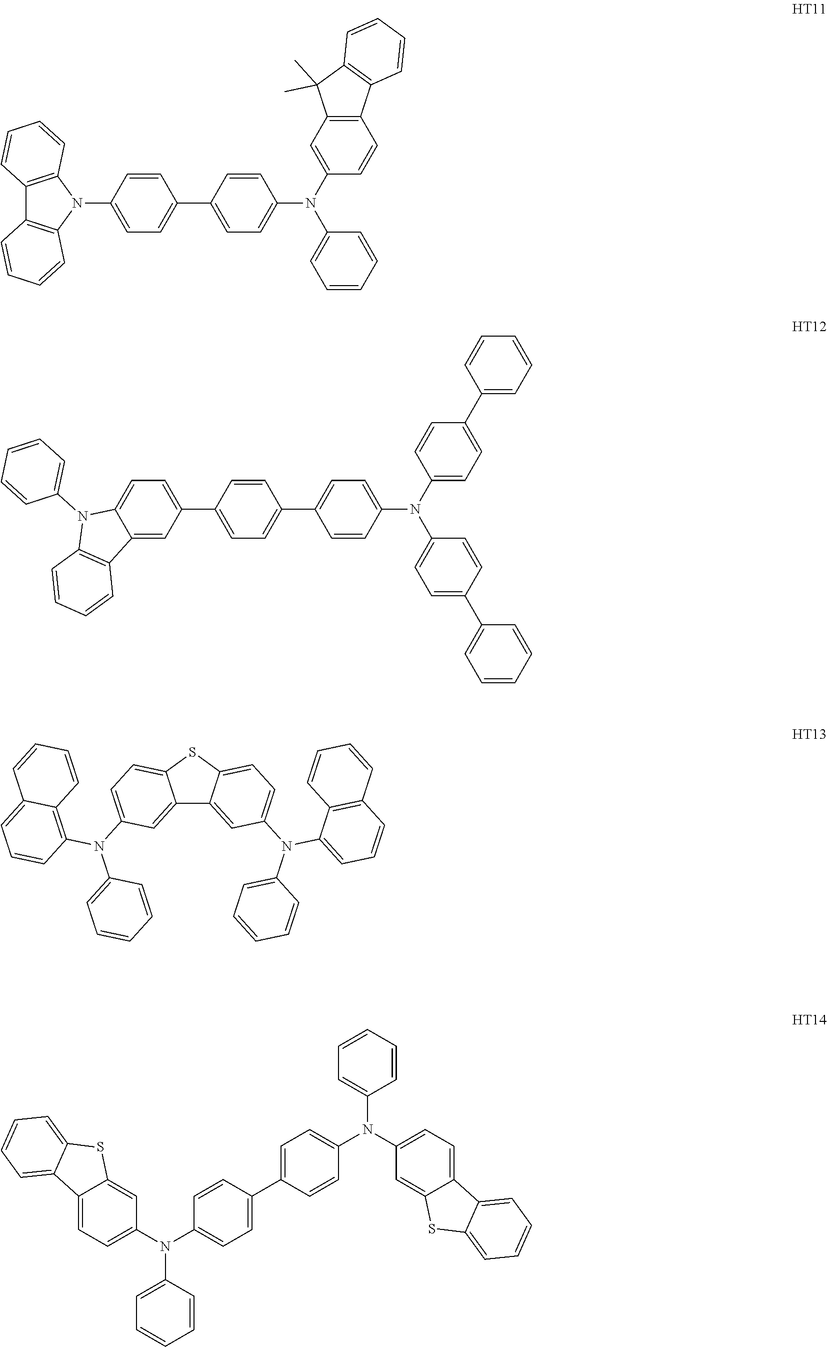 [SMILES: CC1(C)c2ccccc2-c2ccc(N(c3ccccc3)c3ccc(-c4ccc(-n5c6ccccc6c6ccccc65)cc4)cc3)cc21 ; c1ccc(-c2ccc(N(c3ccc(-c4ccccc4)cc3)c3ccc(-c4ccc(-c5ccc6c(c5)c5ccccc5n6-c5ccccc5)cc4)cc3)cc2)cc1 ; c1ccc(N(c2ccc3sc4ccc(N(c5ccccc5)c5cccc6ccccc56)cc4c3c2)c2cccc3ccccc23)cc1 ; c1ccc(N(c2ccc(-c3ccc(N(c4ccccc4)c4ccc5c(c4)sc4ccccc45)cc3)cc2)c2ccc3c(c2)sc2ccccc23)cc1]

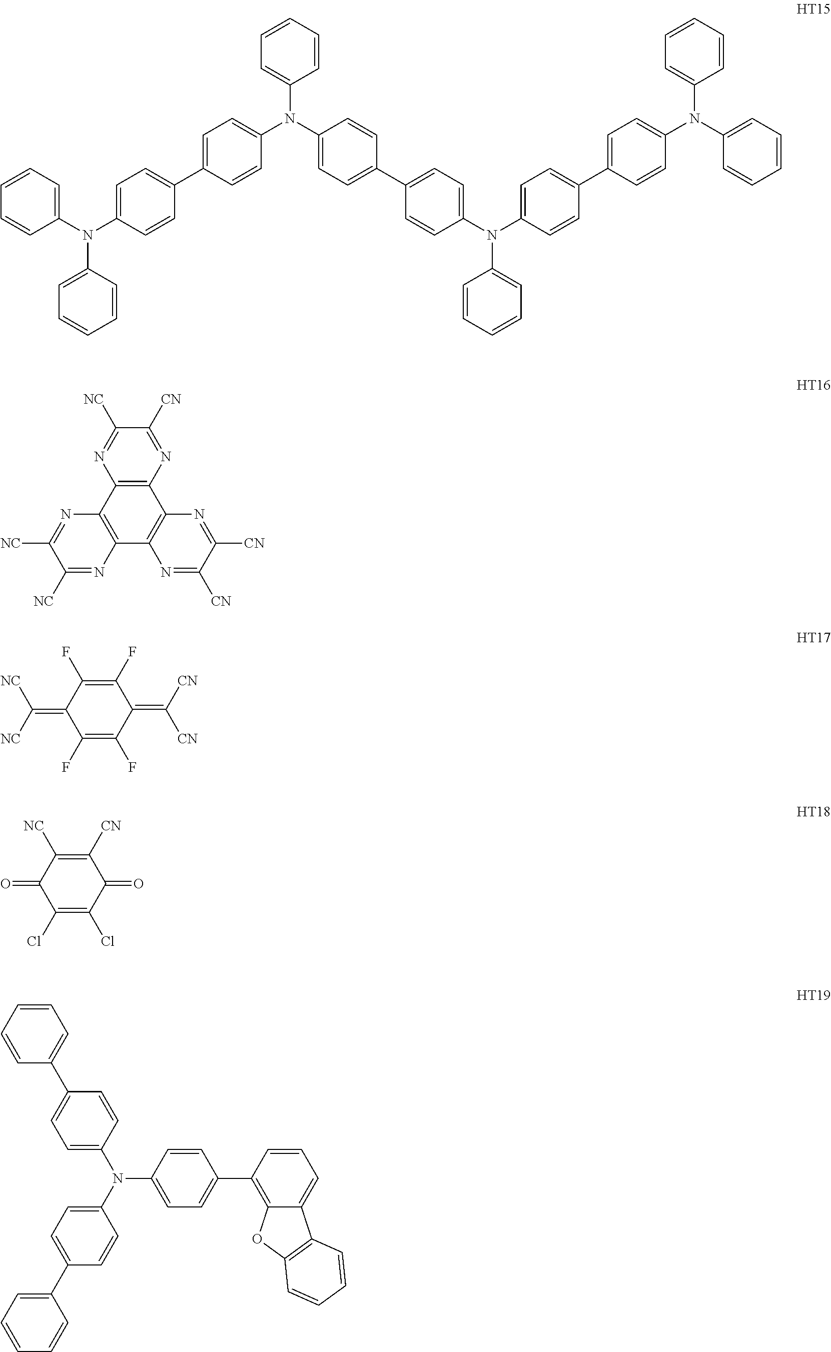 [SMILES: c1ccc(N(c2ccccc2)c2ccc(-c3ccc(N(c4ccccc4)c4ccc(-c5ccc(N(c6ccccc6)c6ccc(-c7ccc(N(c8ccccc8)c8ccccc8)cc7)cc6)cc5)cc4)cc3)cc2)cc1 ; N#Cc1nc2c3nc(C#N)c(C#N)nc3c3nc(C#N)c(C#N)nc3c2nc1C#N ; N#CC(C#N)=c1c(F)c(F)c(=C(C#N)C#N)c(F)c1F ; N#CC1=C(C#N)C(=O)C(Cl)=C(Cl)C1=O ; c1ccc(-c2ccc(N(c3ccc(-c4ccccc4)cc3)c3ccc(-c4cccc5c4oc4ccccc45)cc3)cc2)cc1]

Examples of a light-emitting material mainly associated with a light-emitting function include, in addition to the second organic compound described above, fused-ring compounds, such as fluorene derivatives, naphthalene derivatives, pyrene derivatives, perylene derivatives, tetracene derivatives, anthracene compounds, and rubrene, quinacridone derivatives, coumarin derivatives, stilbene derivatives, organoaluminum complexes, such as tris(8-quinolinolato)aluminum, iridium complexes, platinum complexes, rhenium complexes, copper complexes, europium complexes, ruthenium complexes, and polymer derivatives, such as poly(phenylene vinylene) derivatives, polyfluorene derivatives, and polyphenylene derivatives. Specific examples of a compound that can be used as a light-emitting material are illustrated below, but the light-emitting material is not limited thereto.

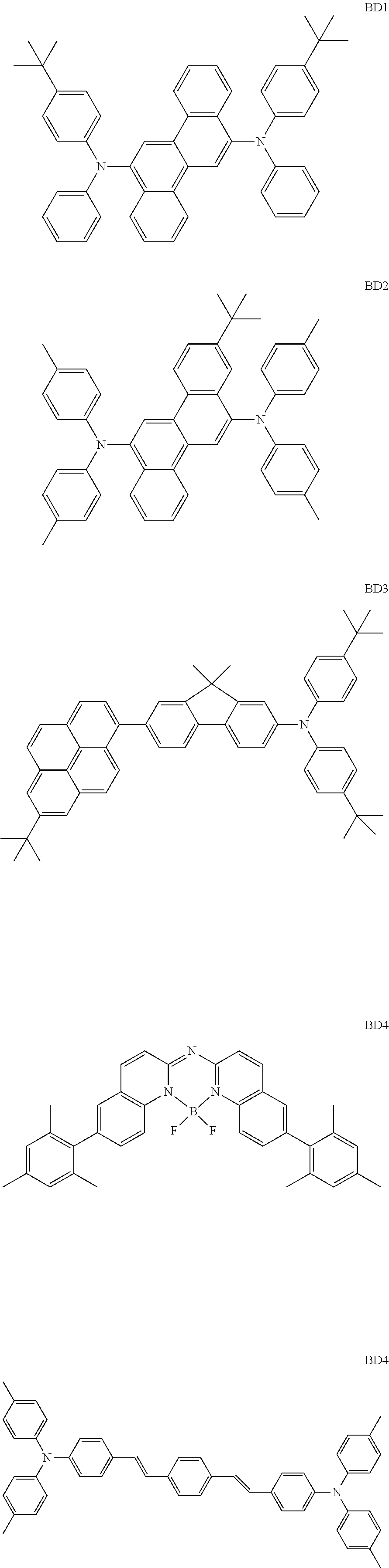

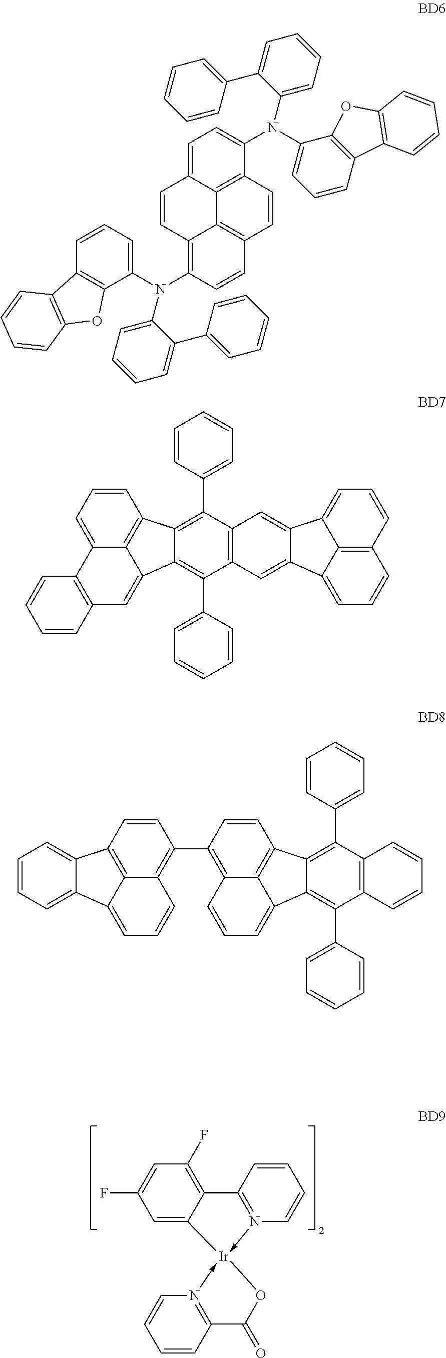

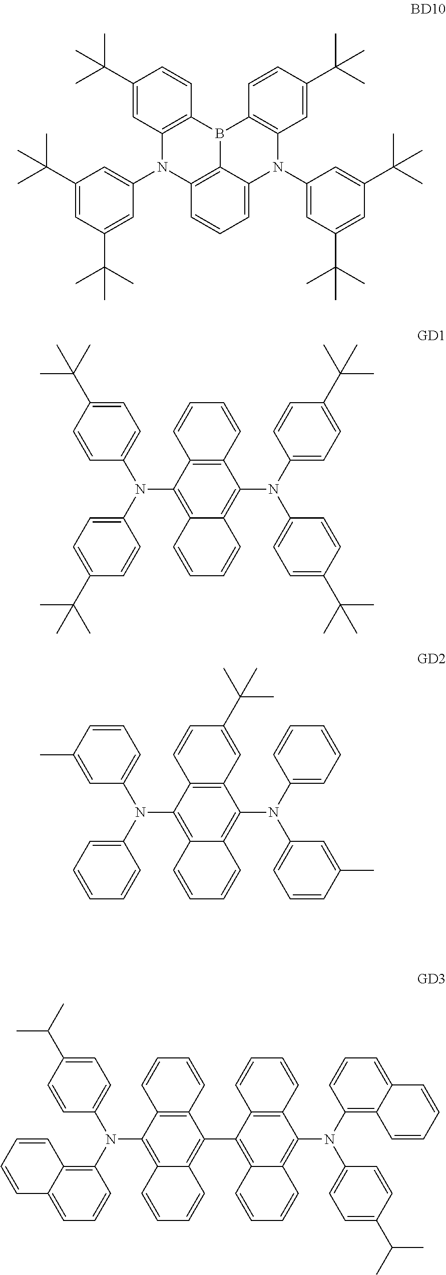

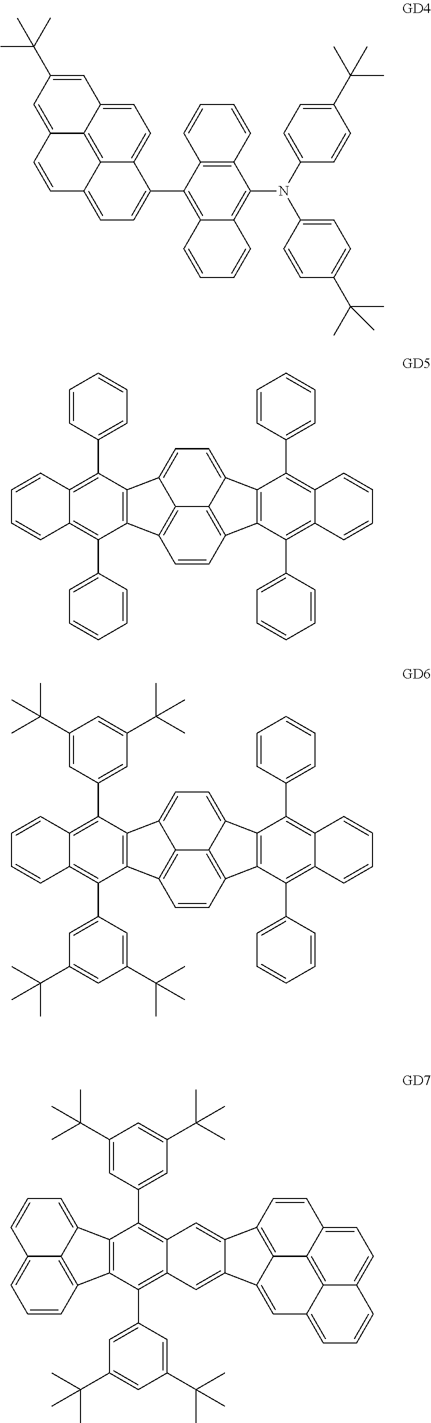

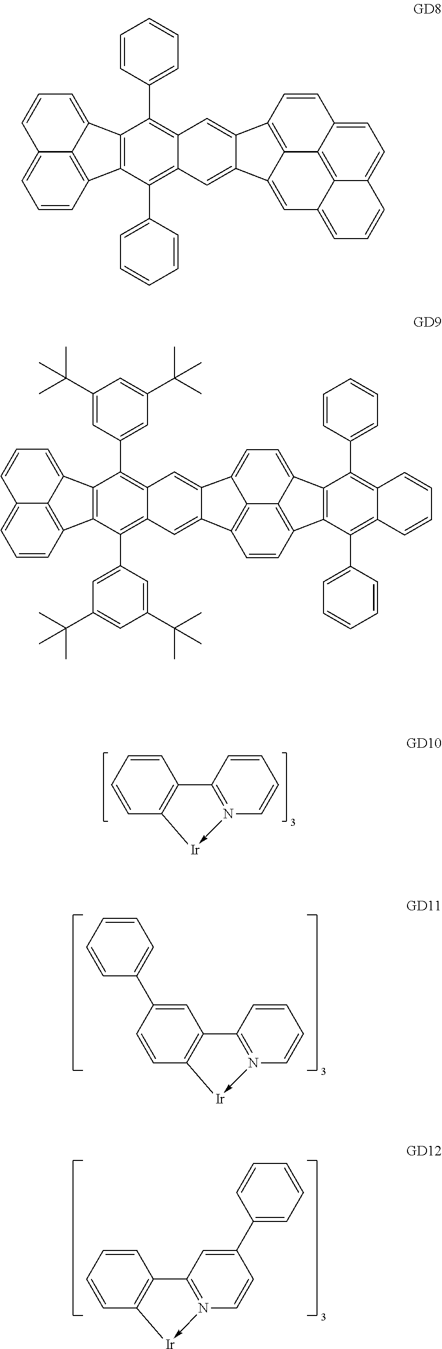

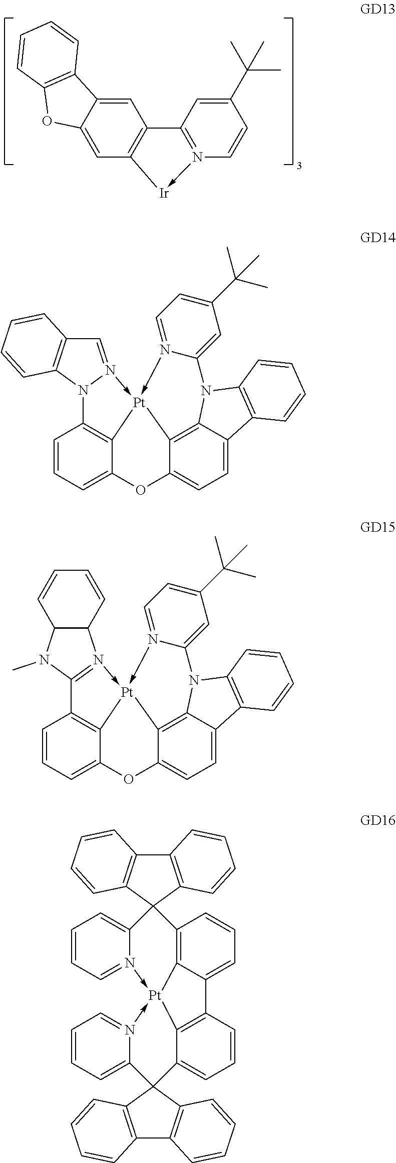

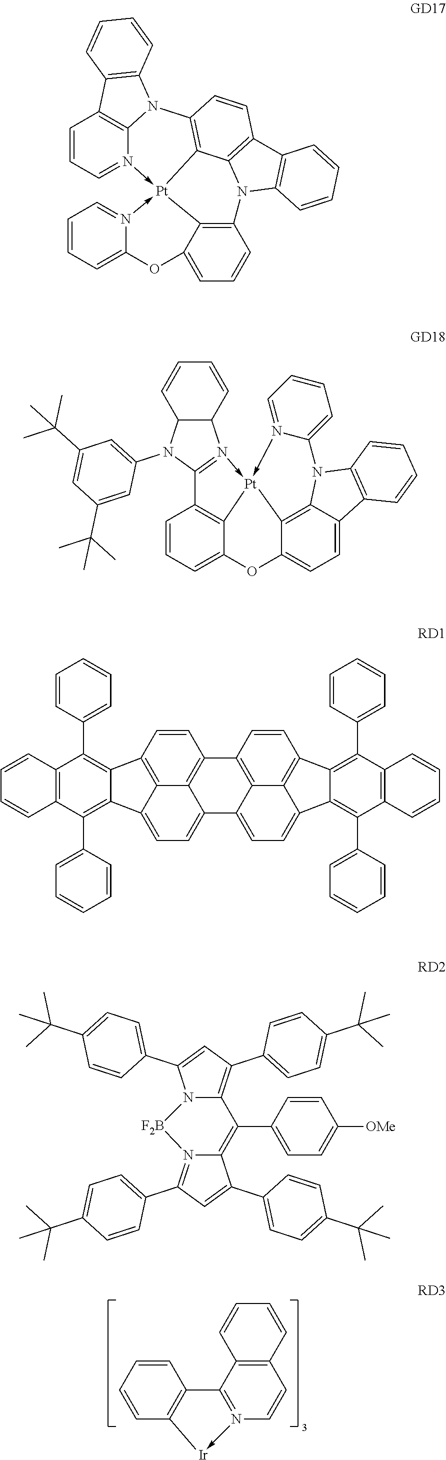

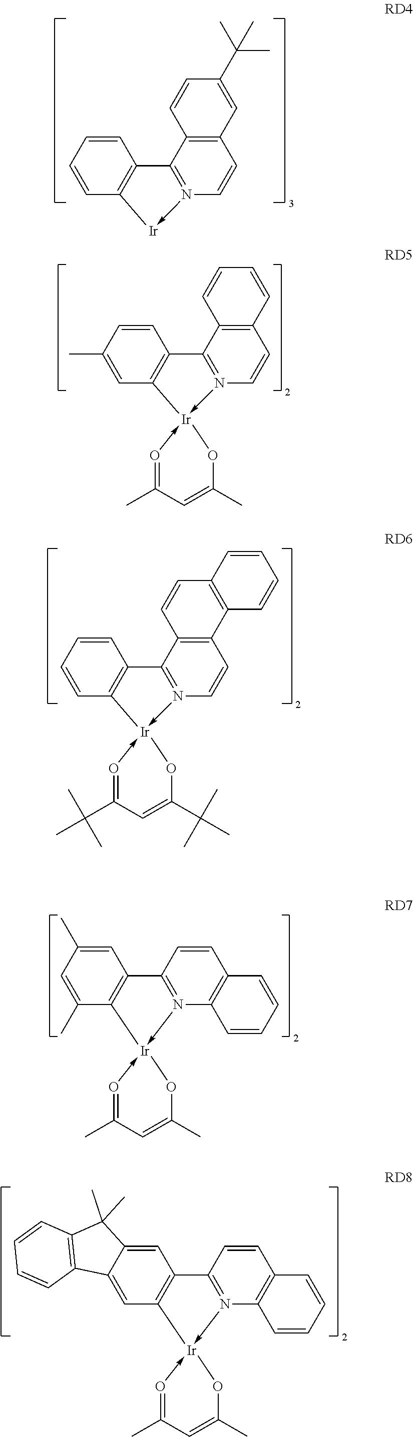

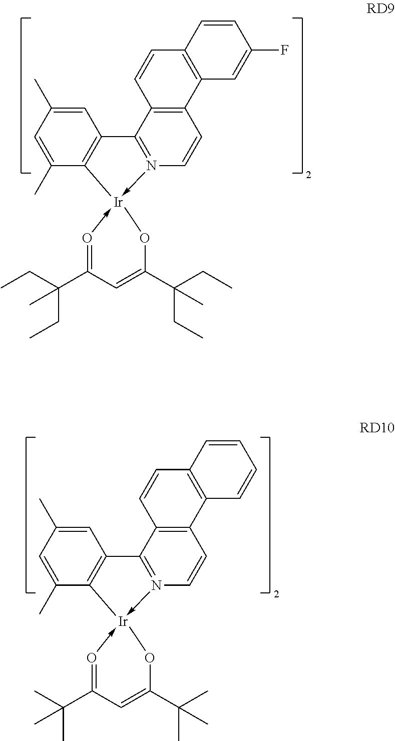

A fourth compound other than the first to third organic compounds may be contained as a host or assist in the light-emitting layer. Examples of the fourth compound include, but are not limited to, aromatic hydrocarbon compounds and derivatives thereof, carbazole derivatives, azine derivatives, xanthone derivatives, dibenzofuran derivatives, dibenzothiophene derivatives, organoaluminum complexes, such as tris(8-quinolinolato)aluminum, and organoberyllium complexes. Specific examples are illustrated below.

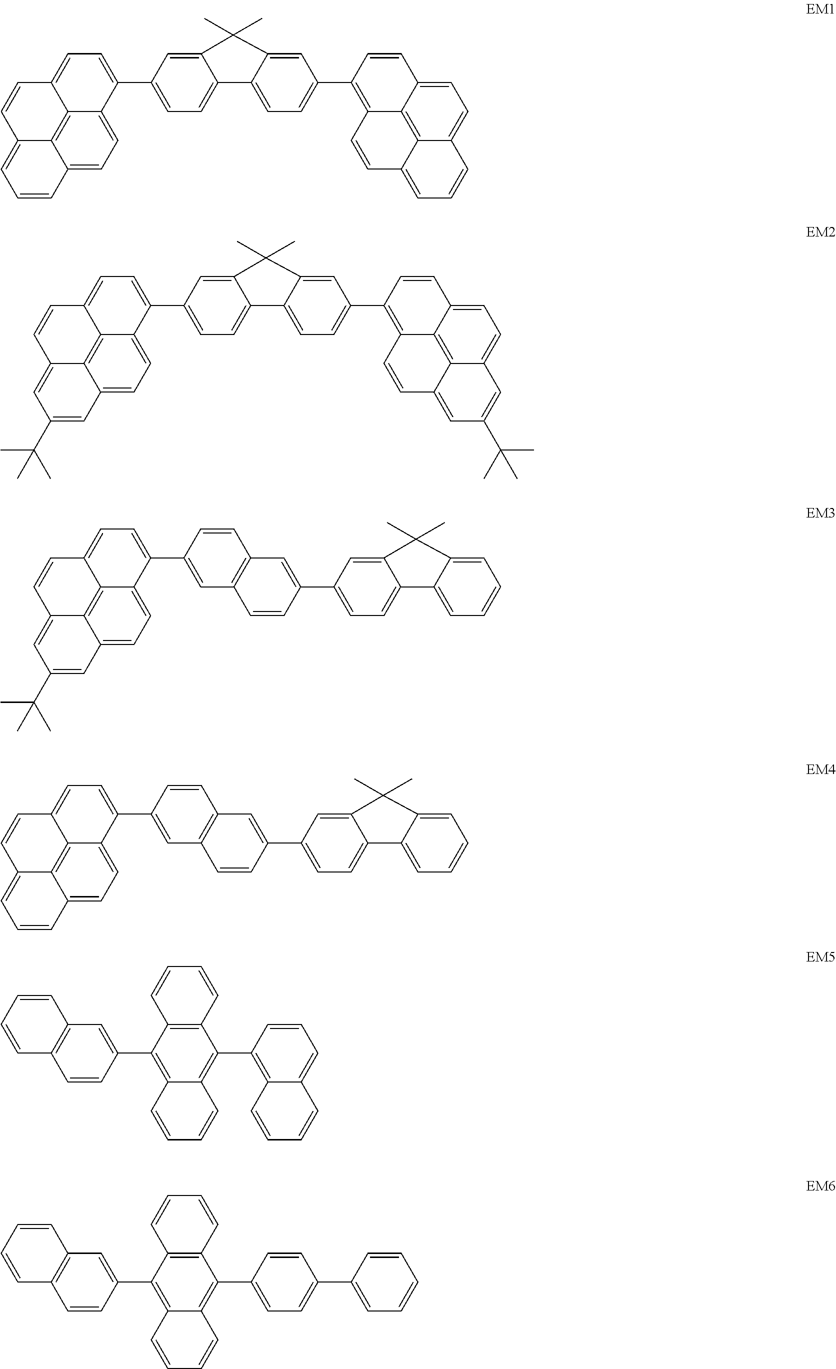

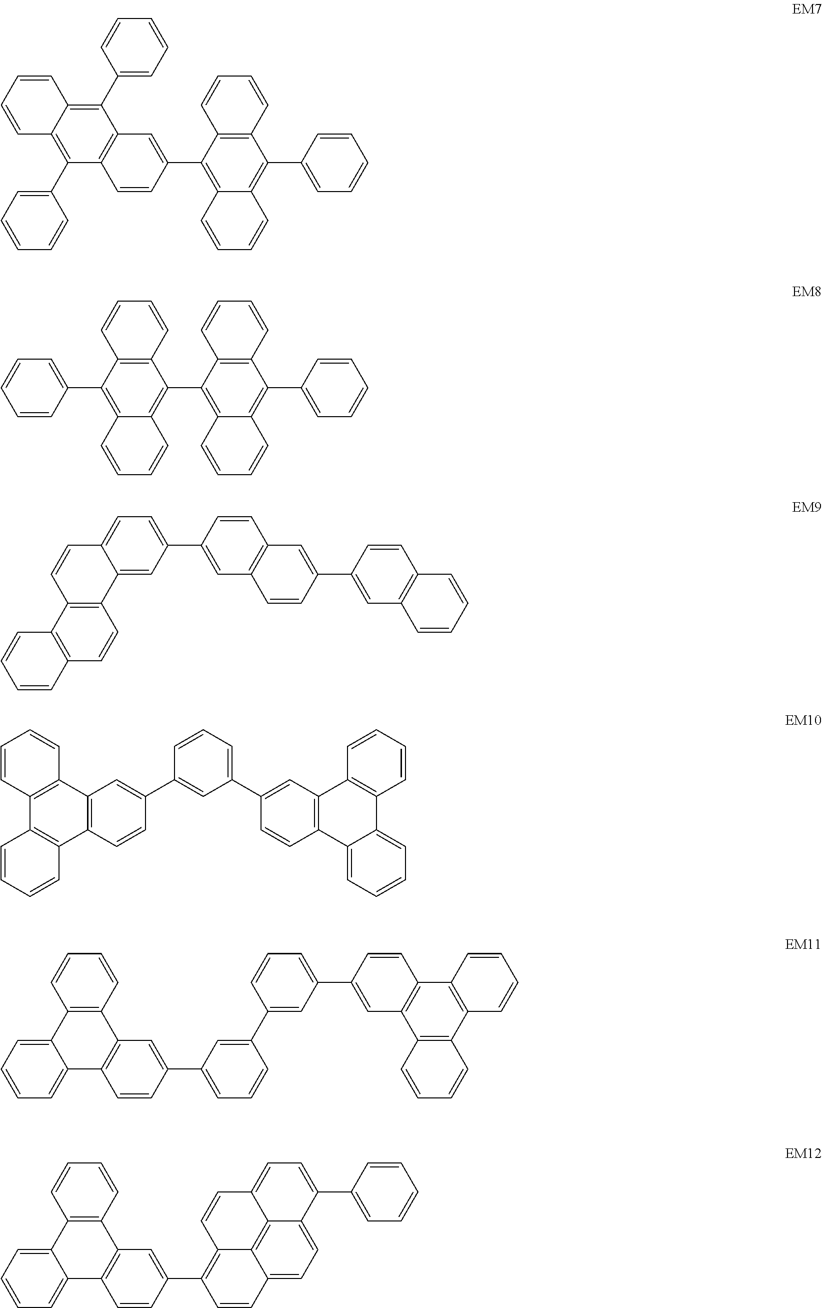

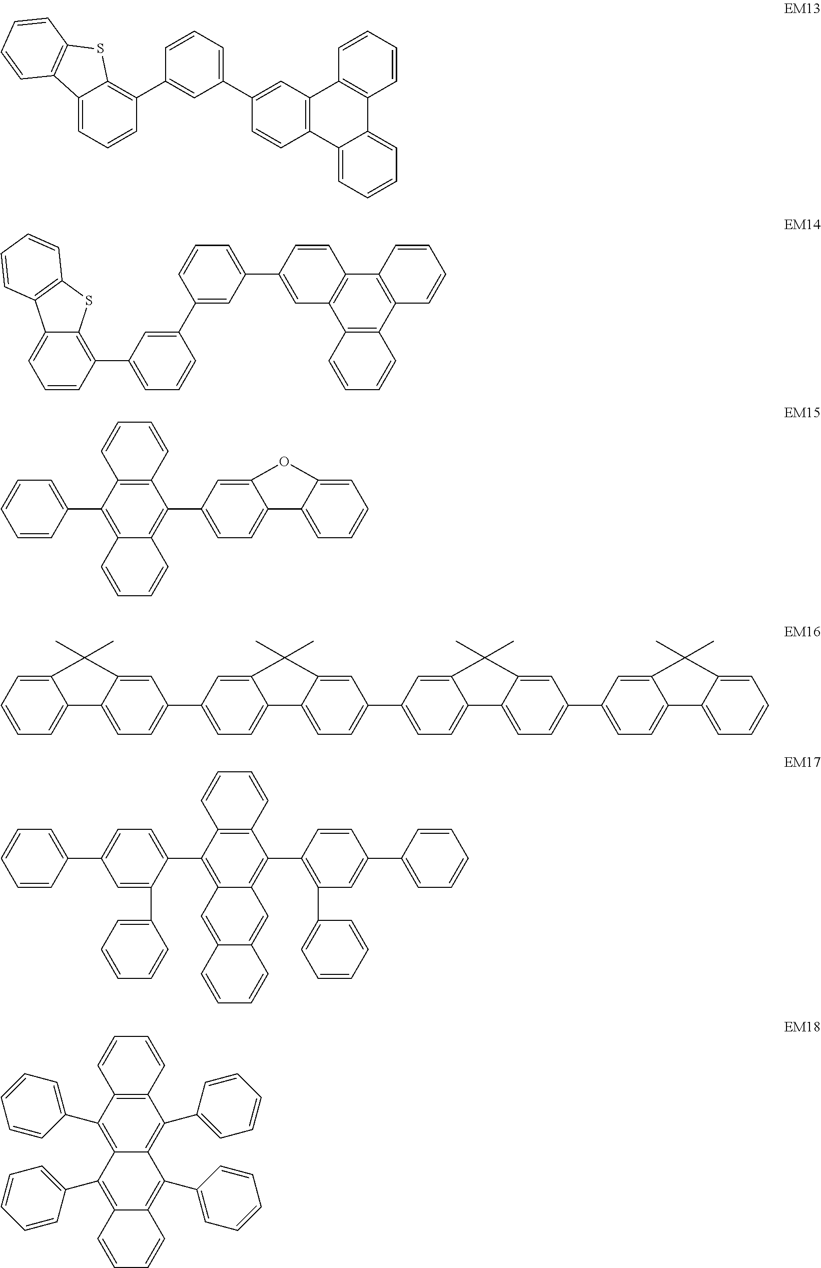

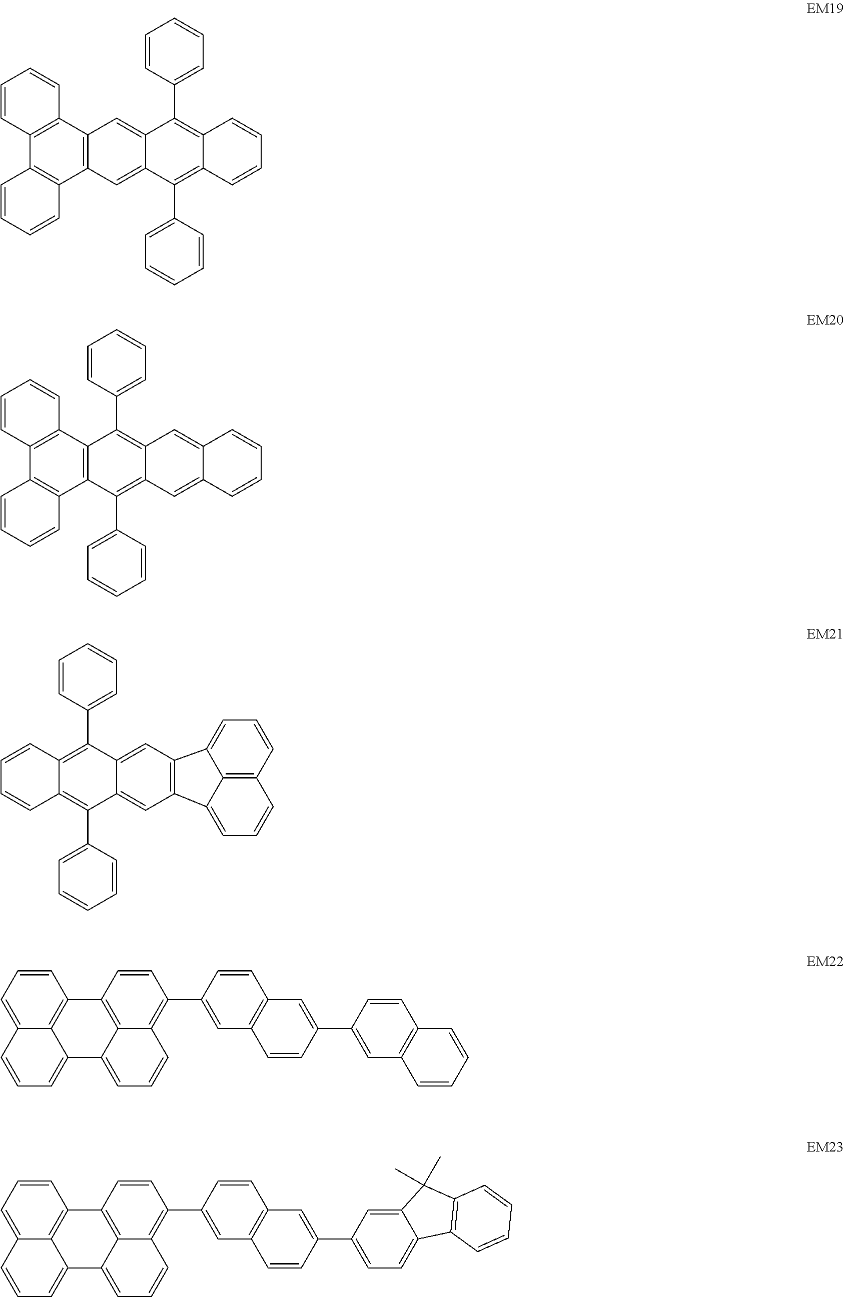

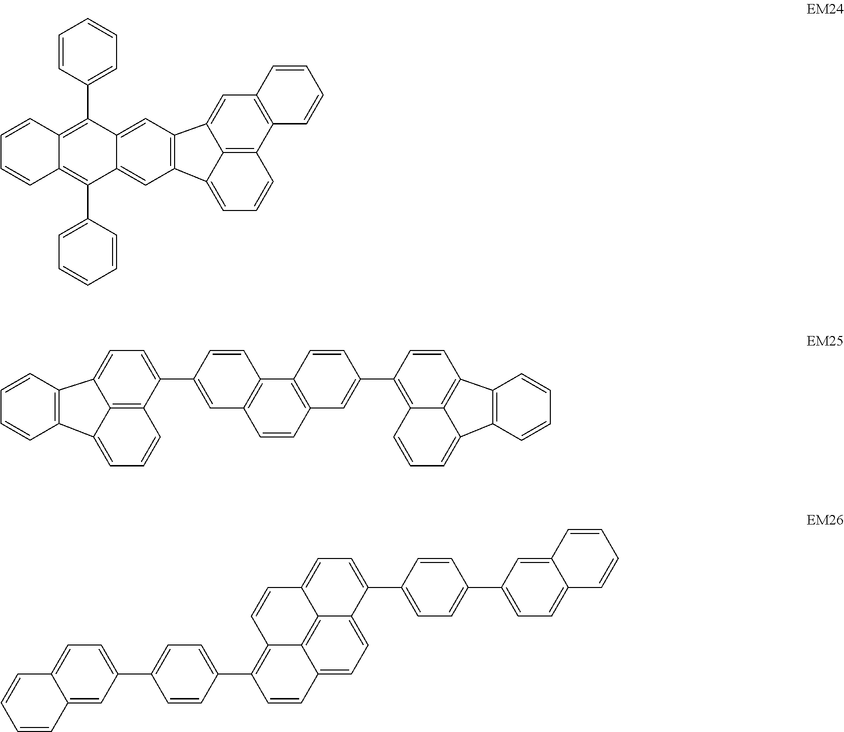

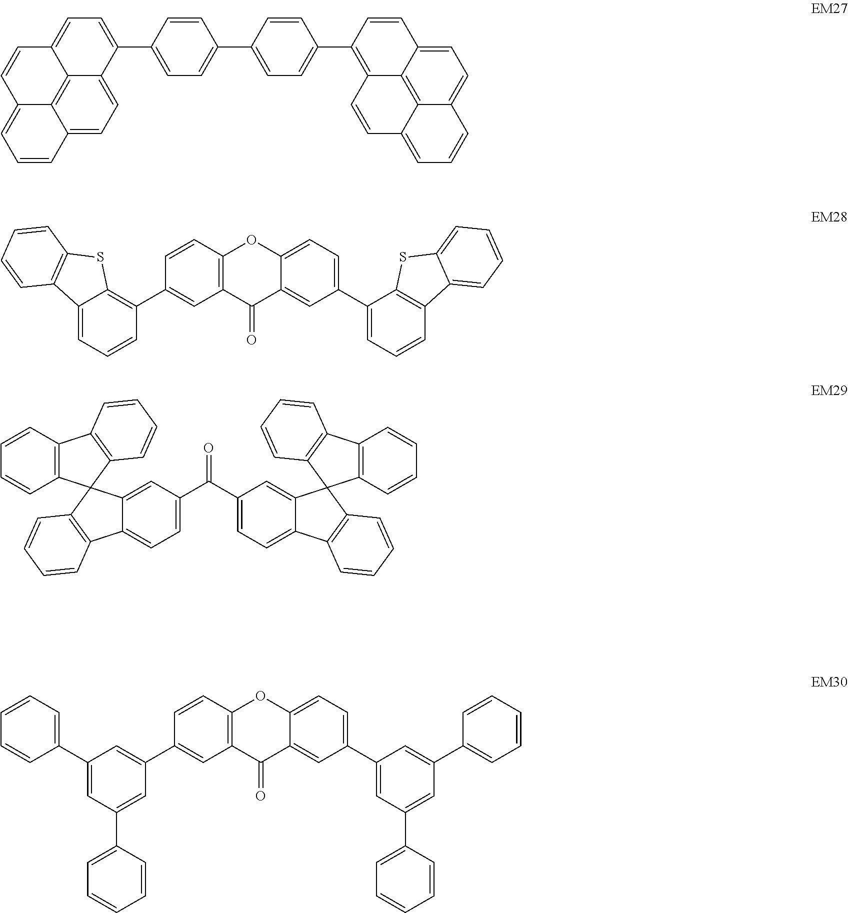

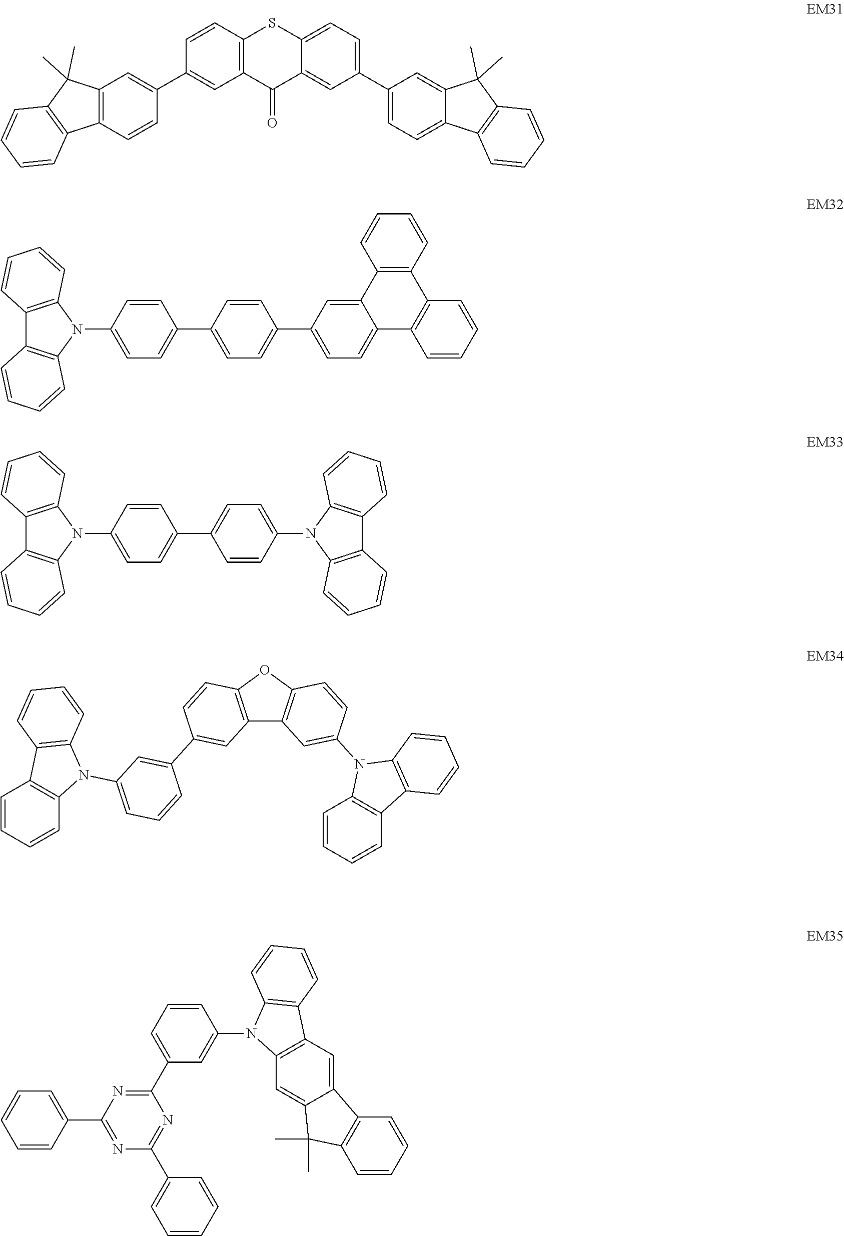

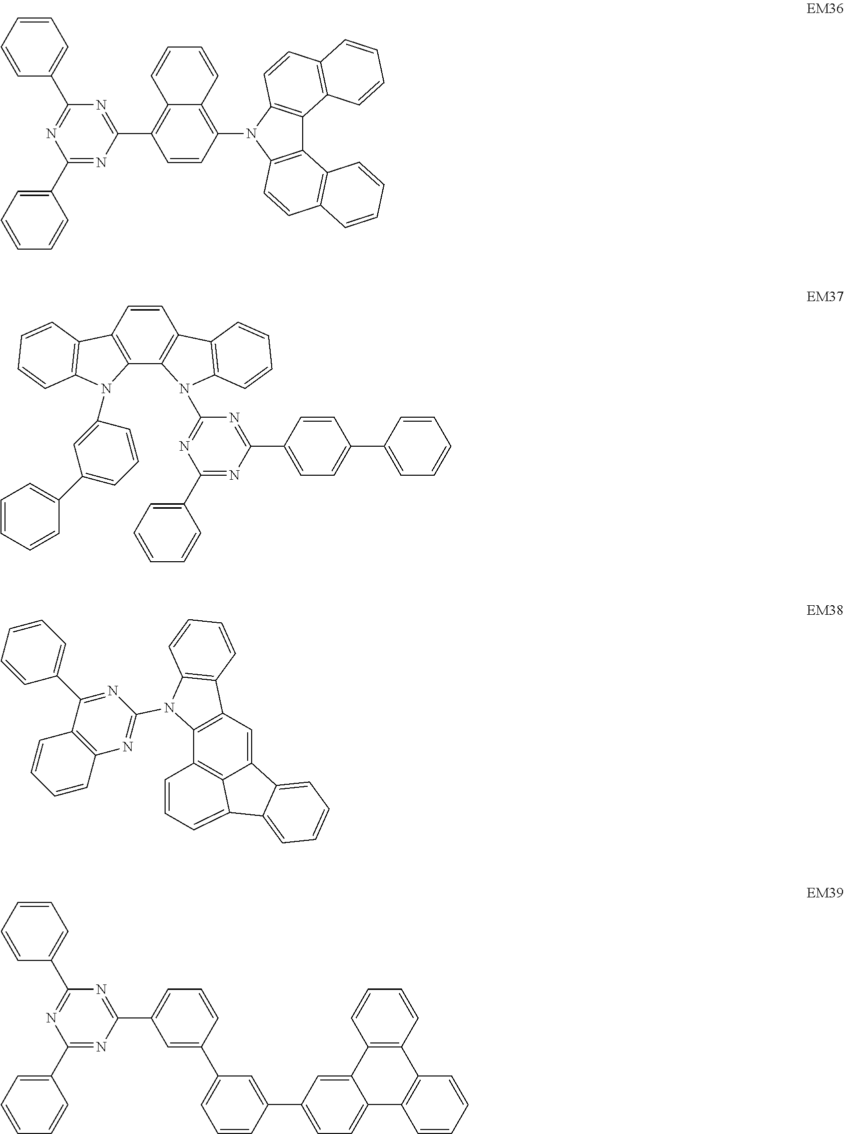 [SMILES: c1ccc(-c2nc(-c3ccccc3)nc(-c3ccc(-n4c5ccc6ccccc6c5c5c6ccccc6ccc54)c4ccccc34)n2)cc1 ; c1ccc(-c2ccc(-c3nc(-c4ccccc4)nc(-n4c5ccccc5c5ccc6c7ccccc7n(-c7cccc(-c8ccccc8)c7)c6c54)n3)cc2)cc1 ; c1ccc(-c2nc(-n3c4ccccc4c4cc5c6c(cccc6c43)-c3ccccc3-5)nc3ccccc23)cc1 ; c1ccc(-c2nc(-c3ccccc3)nc(-c3cccc(-c4cccc(-c5ccc6c7ccccc7c7ccccc7c6c5)c4)c3)n2)cc1]

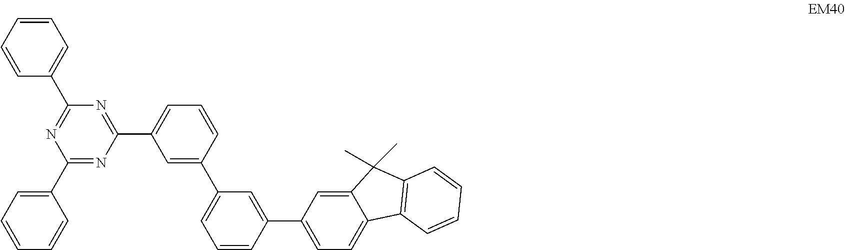 [SMILES: CC1(C)c2ccccc2-c2ccc(-c3cccc(-c4cccc(-c5nc(-c6ccccc6)nc(-c6ccccc6)n5)c4)c3)cc21]

The electron transport material can be freely-selected from materials that can transport electrons injected from the cathode to the light-emitting layer and is selected in consideration of, for example, the balance with the hole mobility of the hole transport material. Examples of a material having the ability to transport electrons include oxadiazole derivatives, oxazole derivatives, pyrazine derivatives, triazole derivatives, triazine derivatives, quinoline derivatives, quinoxaline derivatives, phenanthroline derivatives, organoaluminum complexes, and fused-ring compounds, such as fluorene derivatives, naphthalene derivatives, chrysene derivatives, and anthracene derivatives. The above-described electron transport materials can also be suitably used for the hole-blocking layer. Specific examples of a compound used as the electron transport material are illustrated below, but of course the compound is not limited thereto. Specific examples are illustrated below.

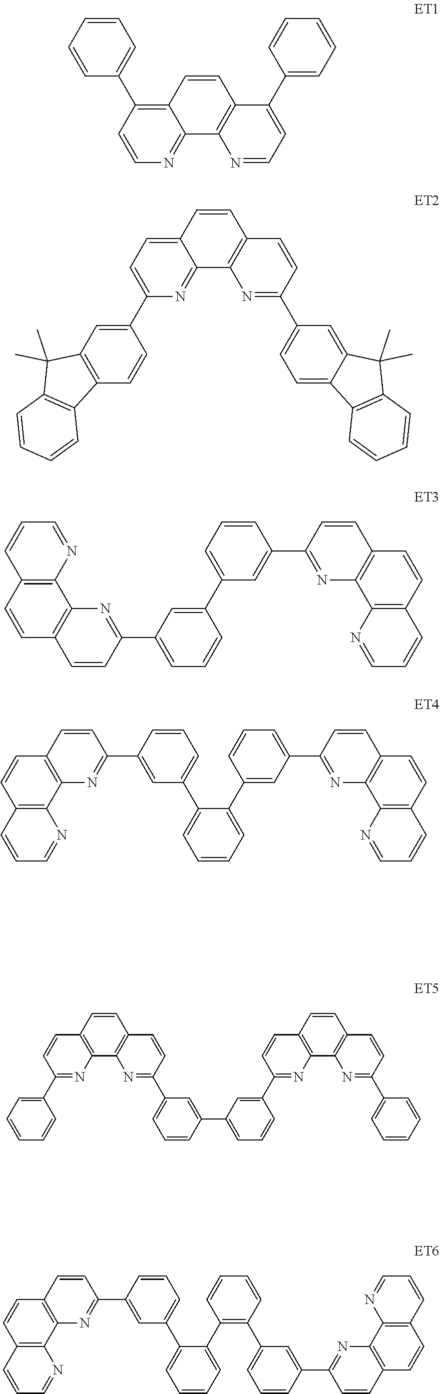

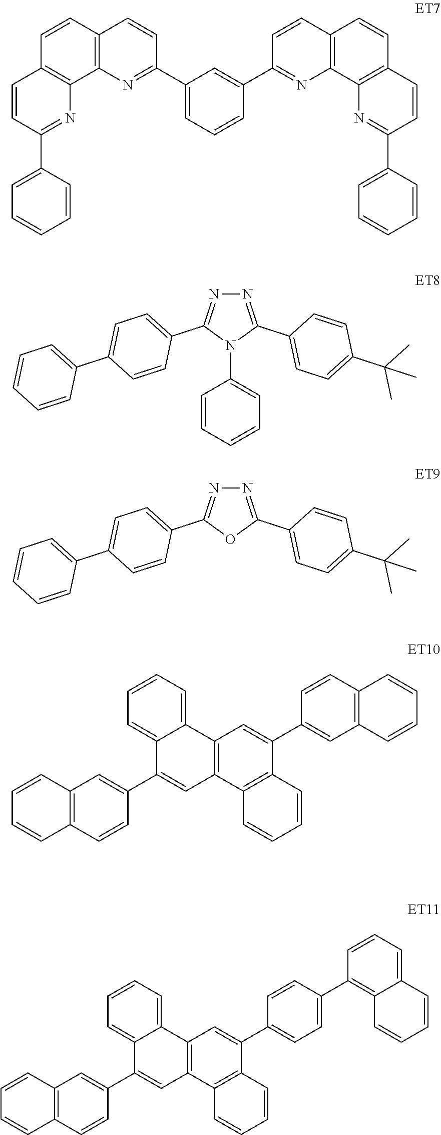

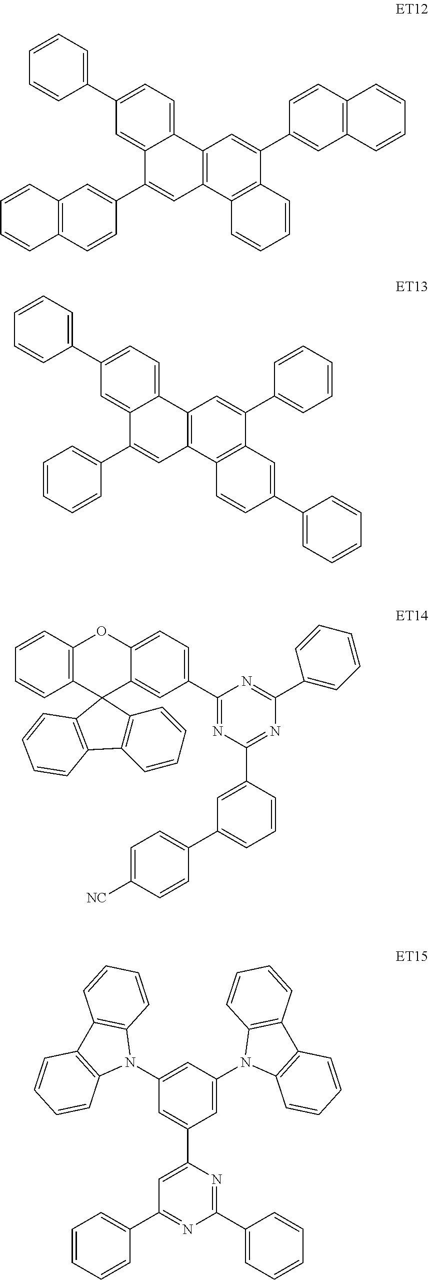

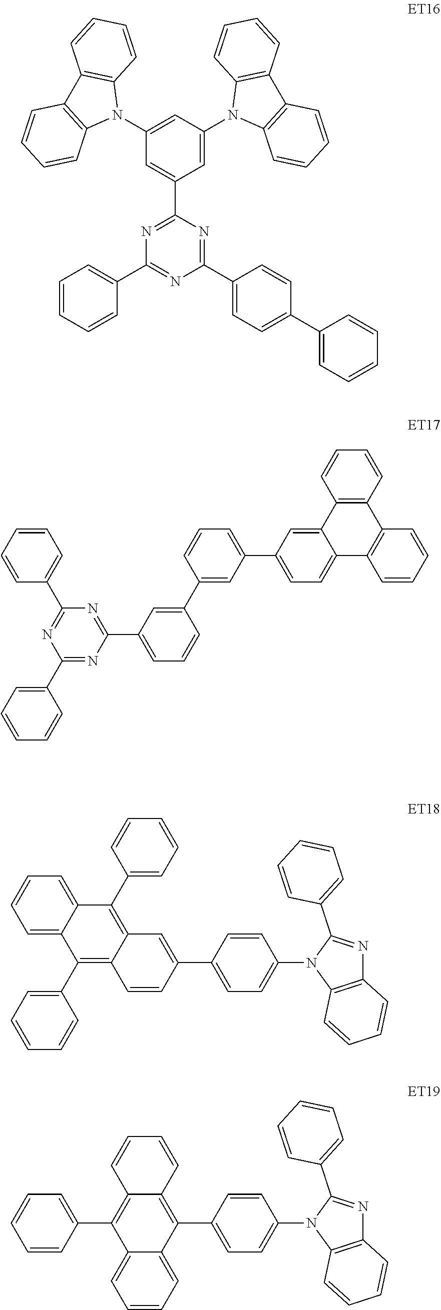 [SMILES: c1ccc(-c2ccc(-c3nc(-c4ccccc4)nc(-c4cc(-n5c6ccccc6c6ccccc65)cc(-n5c6ccccc6c6ccccc65)c4)n3)cc2)cc1 ; c1ccc(-c2nc(-c3ccccc3)nc(-c3cccc(-c4cccc(-c5ccc6c7ccccc7c7ccccc7c6c5)c4)c3)n2)cc1 ; c1ccc(-c2c3ccccc3c(-c3ccccc3)c3cc(-c4ccc(-n5c(-c6ccccc6)nc6ccccc65)cc4)ccc23)cc1 ; c1ccc(-c2c3ccccc3c(-c3ccc(-n4c(-c5ccccc5)nc5ccccc54)cc3)c3ccccc23)cc1]

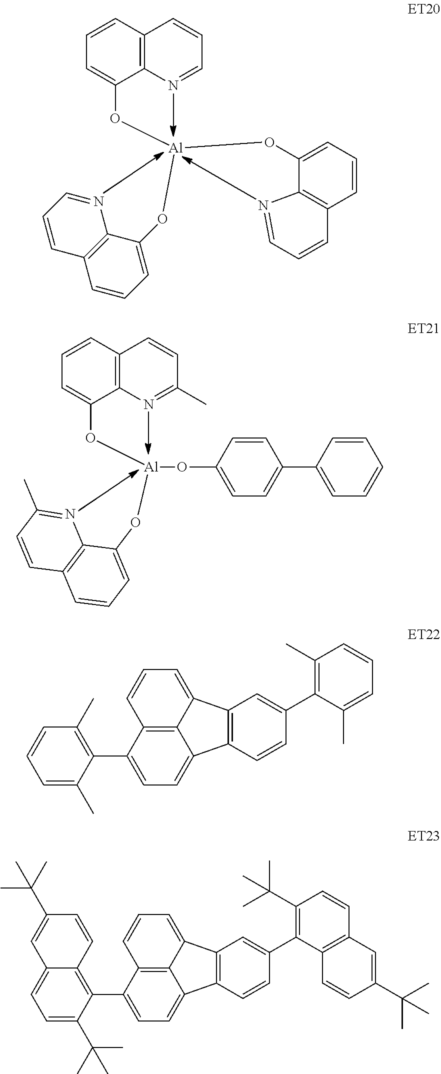

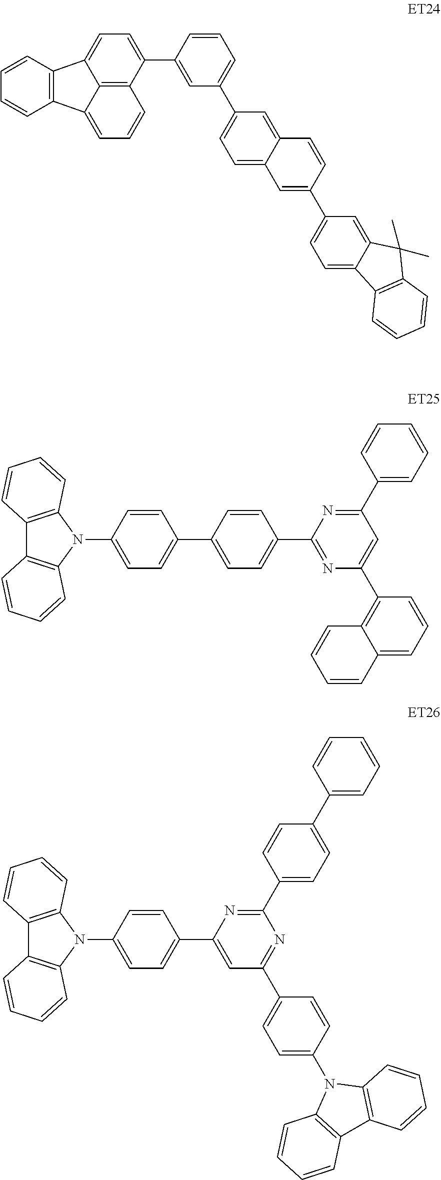

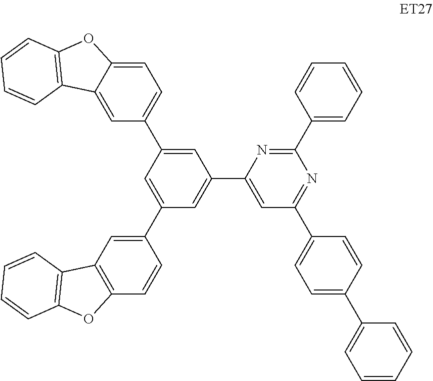 [SMILES: c1ccc(-c2ccc(-c3cc(-c4cc(-c5ccc6oc7ccccc7c6c5)cc(-c5ccc6oc7ccccc7c6c5)c4)nc(-c4ccccc4)n3)cc2)cc1]

An electron injection material can be freely-selected from materials into which electrons can be easily injected from the cathode and is selected in consideration of, for example, the balance with the hole-injecting property. As the organic compound, n-type dopants and reducing dopants are also included. Examples thereof include alkali metal-containing compounds, such as lithium fluoride, lithium complexes, such as lithium quinolinolate, benzimidazolidene derivatives, imidazolidene derivatives, fulvalene derivatives, and acridine derivatives. It can also be used in combination with the above-mentioned electron transport material.

### Configuration of Organic Light-Emitting Device

The organic light-emitting device includes, over a substrate, an insulating layer, a first electrode, organic compound layers, and a second electrode. A protective layer, a color filter, a microlens, and so forth may be disposed over the second electrode. In the case of disposing the color filter, a planarization layer may be disposed between the protective layer and the color filter. The planarization layer can be composed of, for example, an acrylic resin. The same applies when a planarization layer is provided between the color filter and the microlens.

### Substrate

Examples of the substrate include quartz, glass, silicon wafers, resins, and metals. The substrate may include a switching element, such as a transistor, a line, and an insulating layer thereon. Any material can be used for the insulating layer as long as a contact hole can be formed in such a manner that a line can be coupled to the first electrode and as long as insulation with a non-connected line can be ensured. For example, a resin, such as polyimide, silicon oxide, or silicon nitride, can be used.

### Electrode

As the electrodes, a pair of electrodes can be used. The pair of electrodes may be an anode and a cathode. When an electric field is applied in the direction in which the organic light-emitting device emits light, an electrode having a higher potential is the anode, and the other is the cathode. It can also be said that the electrode that supplies holes to the light-emitting layer is the anode and that the electrode that supplies electrons is the cathode.

As a constituent material of the anode, a material having a work function as large as possible is preferred. Examples of the material that can be used include elemental metals, such as gold, platinum, silver, copper, nickel, palladium, cobalt, selenium, vanadium, and tungsten, mixtures thereof, alloys of combinations thereof, and metal oxides, such as tin oxide, zinc oxide, indium oxide, indium-tin oxide (ITO), and indium-zinc oxide. Additionally, conductive polymers, such as polyaniline, polypyrrole, and polythiophene, can also be used.

These electrode materials may be used alone or in combination of two or more. The anode may be formed of a single layer or multiple layers.

When the electrode is used as a reflective electrode, for example, chromium, aluminum, silver, titanium, tungsten, molybdenum, an alloy thereof, or a stack thereof can be used. These materials can also be used to act as a reflective film that does not have the role of an electrode. When the electrode is used as a transparent electrode, a transparent conductive oxide layer composed of, for example, indium-tin oxide (ITO) or indium-zinc oxide can be used; however, the electrode is not limited thereto. The electrode can be formed by a photolithography technique.

As a constituent material of the cathode, a material having a small work function is preferred. Examples thereof include elemental metals, such as alkali metals, e.g., lithium, alkaline-earth metals, e.g., calcium, aluminum, titanium, manganese, silver, lead, and chromium, and mixtures containing them. Alternatively, an alloy made by combining these metal elements can also be used. For example, magnesium-silver, aluminum-lithium, aluminum-magnesium, silver-copper, zinc-silver, and so forth can be used. Metal oxides, such as indium-tin oxide (ITO), can also be used. These electrode materials may be used alone or in combination of two or more. The cathode may have a single-layer structure or a multilayer structure. In particular, silver is preferably used. To reduce the aggregation of silver, a silver alloy is more preferred. Any alloy ratio may be used as long as the aggregation of silver can be reduced. The ratio of silver to another metal may be, for example, 1:1 or 3:1.

A top emission device may be provided using the cathode formed of a conductive oxide layer composed of, for example, ITO. A bottom emission device may be provided using the cathode formed of a reflective electrode composed of, for example, aluminum (Al). The cathode is not particularly limited. A method for forming the cathode is not particularly limited, but a direct-current sputtering method, an alternating-current sputtering method, or the like is more preferably used because good film coverage is obtained and thus the resistance is easily reduced.

### Organic Compound Layer

The organic compound layer includes at least the light-emitting layer, and may include, in addition to the light-emitting layer, the hole injection layer, the hole transport layer, and the electron-blocking layer on the anode side, and the hole-blocking layer, the electron transport layer, an electron injection layer, and the like on the cathode side, which are appropriately selected as necessary. The organic compound layer is mainly composed of an organic compound, and may contain inorganic atoms and an inorganic compound. For example, each organic compound layer may contain, for example, copper, lithium, magnesium, aluminum, iridium, platinum, molybdenum, or zinc.

For the organic compound layer included in the organic light-emitting device according to an embodiment of the present invention, a dry process, such as a vacuum evaporation method, an ionized evaporation method, sputtering, or plasma, can be employed. Alternatively, instead of the dry process, it is also possible to employ a wet process in which a material is dissolved in an appropriate solvent and then a film is formed by a known coating method (for example, spin coating, dipping, a casting method, an LB technique, or an ink jet method).

When the layer is formed by, for example, the vacuum evaporation method or the solution coating method, crystallization and so forth are less likely to occur, and good stability with time is obtained. In the case of forming a film by the coating method, the film can be formed in combination with an appropriate binder resin.

Examples of the binder resin include, but are not limited to, polyvinyl carbazole resins, polycarbonate resins, polyester resins, ABS resins, acrylic resins, polyimide resins, phenolic resins, epoxy resins, silicon resins, and urea resins.

These binder resins may be used alone as a homopolymer or copolymer or in combination as a mixture of two or more. Furthermore, additives, such as a known plasticizer, antioxidant, and ultraviolet absorber, may be used, as needed.

### Protective Layer

A protective layer may be disposed on the second electrode. For example, a glass member provided with a moisture absorbent can be bonded to the second electrode to reduce the entry of, for example, water into the organic compound layer, thereby reducing the occurrence of display defects. In another embodiment, a passivation film composed of, for example, silicon nitride may be disposed on the second electrode to reduce the entry of, for example, water into the organic compound layer. For example, after the formation of the second electrode, the substrate may be transported to another chamber without breaking the vacuum, and a silicon nitride film having a thickness of 2 μm may be formed by a chemical vapor deposition (CVD) method to provide a protective layer. After the film deposition by the CVD method, a protective layer may be formed by an atomic layer deposition (ALD) method. Examples of the material of the layer formed by the ALD method may include, but are not limited to, silicon nitride, silicon oxide, and aluminum oxide. Silicon nitride may be deposited by the CVD method on the layer formed by the ALD method. The film formed by the ALD method may have a smaller thickness than the film formed by the CVD method. Specifically, the thickness may be 50% or less, even 10% or less.

### Color Filter

A color filter may be disposed on the protective layer. For example, a color filter may be disposed on another substrate in consideration of the size of the organic light-emitting device and bonded to the substrate provided with the organic light-emitting device. A color filter may be formed by patterning on the protective layer using a photolithography technique. The color filter may be composed of a polymer.

### Planarization Layer

A planarization layer may be disposed between the color filter and the protective layer. The planarization layer is provided for the purpose of reducing the unevenness of the layer underneath. The planarization layer may be referred to as a “material resin layer” without limiting its purpose. The planarization layer may be composed of an organic compound, may have a low-molecular-weight or high-molecular-weight compound, and is preferably a high-molecular-weight compound.

The planarization layers may be disposed above and below the color filter and may be composed of the same or different constituent materials. Specific examples thereof include polyvinyl carbazole resins, polycarbonate resins, polyester resins, ABS resins, acrylic resins, polyimide resins, phenolic resins, epoxy resins, silicone resins, and urea resins.

### Microlens

The organic light-emitting device or a light-emitting apparatus including the organic light-emitting device may include an optical member, such as a microlens, on the outgoing light side. The microlens can be composed of, for example, an acrylic resin or an epoxy resin. The microlens may be used to increase the amount of light emitted from the organic light-emitting device or the organic light-emitting apparatus and to control the direction of the light emitted. The microlens may have a hemispherical shape. In the case of a hemispherical shape, among tangents to the hemisphere, there is a tangent parallel to the insulating layer. The point of contact of the tangent with the hemisphere is the vertex of the microlens. The vertex of the microlens can be determined in the same way for any cross-sectional view. That is, among the tangents to the semicircle of the microlens in the cross-sectional view, there is a tangent parallel to the insulating layer, and the point of contact of the tangent with the semicircle is the vertex of the microlens.

The midpoint of the microlens can be defined. In the cross section of the microlens, when a segment is hypothetically drawn from the point where an arc shape ends to the point where another arc shape ends, the midpoint of the segment can be referred to as the midpoint of the microlens. The cross section to determine the vertex and midpoint may be a cross section perpendicular to the insulating layer.

### Opposite Substrate

An opposite substrate may be disposed on the planarization layer. The opposite substrate is disposed at a position corresponding to the substrate described above and thus is called an opposite substrate. The opposite substrate may be composed of the same constituent material as that of the substrate described above. When the above-described substrate is referred to as a first substrate, the opposite substrate may be referred to as a second substrate.

### Pixel Circuit

The light-emitting apparatus including organic light-emitting devices may include pixel circuits coupled to the organic light-emitting devices. Each of the pixel circuits may be of an active matrix type, which independently controls the emission of multiple organic light-emitting devices. The active matrix type circuit may be voltage programming or current programming. A driving circuit includes the pixel circuit for each pixel. The pixel circuit may include the organic light-emitting device, a transistor to control the luminance of the organic light-emitting device, a transistor to control the timing of the light emission, a capacitor to retain the gate voltage of the transistor to control the luminance, and a transistor to connect to GND without using the light-emitting device.

The light-emitting apparatus includes a display area and a peripheral area disposed around the display area. The display area includes a pixel circuit, and the peripheral area includes a display control circuit. The mobility of a transistor contained in the pixel circuit may be lower than the mobility of a transistor contained in the display control circuit. The slope of the current-voltage characteristics of the transistor contained in the pixel circuit may be smaller than the slope of the current-voltage characteristic of the transistor contained in the display control circuit. The slope of the current-voltage characteristics can be measured by what is called Vg-Ig characteristics. The transistor contained in the pixel circuit is a transistor coupled to an organic light-emitting device.

### Pixel

A light-emitting apparatus including an organic light-emitting device may include multiple pixels. Each pixel includes subpixels configured to emit colors different from each other. The subpixels may have respective RGB emission colors.

Light emerges from a region of the pixel, also called a pixel aperture. This region is also referred to as a “first region”. The pixel aperture may be 15 μm or less, and may be 5 μm or more. More specifically, the pixel aperture may be, for example, 11 μm, 9.5 μm, 7.4 μm, or 6.4 μm. The distance between subpixels may be 10 μm or less. Specifically, the distance may be 8 μm, 7.4 μm, or 6.4 μm.

The pixels may be arranged in a known configuration in plan view. For example, a stripe pattern, a delta pattern, a Pen Tile matrix pattern, or the Bayer pattern may be used. The shape of each subpixel in plan view may be any known shape. Examples thereof include quadrilaterals, such as rectangles and rhombi, and hexagons. Of course, if the shape is close to a rectangle, rather than an exact shape, it is included in the rectangle. The shape of the subpixel and the pixel arrangement can be used in combination.

### Application of Organic Light-Emitting Device

The organic light-emitting device according to the present embodiment can be used as a component member of a display apparatus or lighting apparatus. Other applications include exposure light sources for electrophotographic image-forming apparatuses, backlights for liquid crystal display apparatuses, and light-emitting apparatuses including white light sources provided with color filters.

The display apparatus may be an image information-processing apparatus including an image input unit that receives image information from an area CCD, a linear CCD, a memory card, or the like, an information-processing unit that processes the input information, and a display unit that displays the input image. The display apparatus includes multiple pixels, and at least one of the multiple pixels may include the organic light-emitting device of the present embodiment and a transistor coupled to the organic light-emitting device.

The display unit of an image pickup apparatus or an ink jet printer may have a touch panel function. The driving mode of the touch panel function may be, but is not particularly limited to, an infrared mode, an electrostatic capacitance mode, a resistive film mode, or an electromagnetic inductive mode. The display apparatus may also be used for a display unit of a multifunction printer.

The following describes a display apparatus according to the present embodiment with reference to the attached drawings.

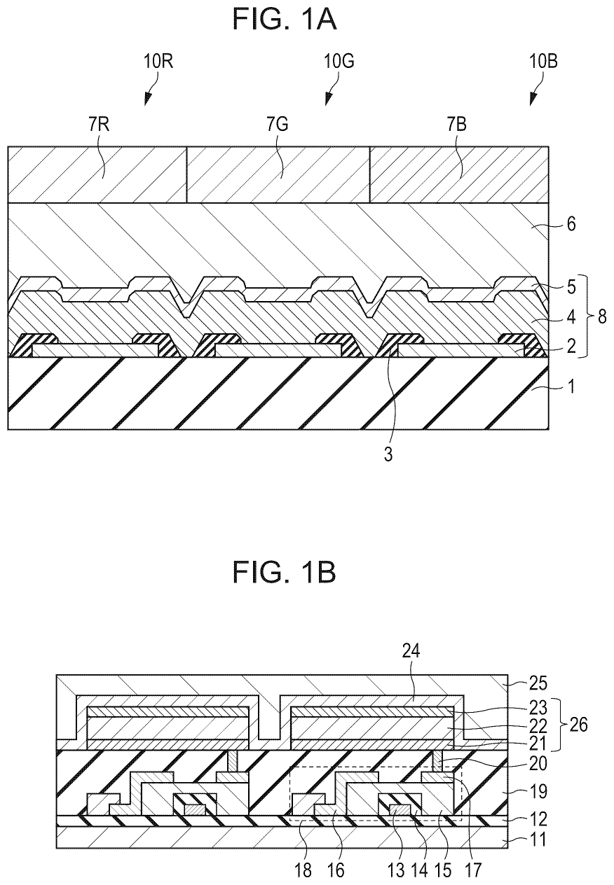

FIG. 1A illustrates an example of pixels that are constitutional elements of the display apparatus according to the present embodiment. The pixel includes subpixels 10. The subpixels are divided into 10R, 10G, and 10B according to their light emission. The emission colors may be distinguished by the wavelength of light emitted from the light-emitting layer. Light emitted from the subpixels may be selectively transmitted or color-converted with, for example, a color filter. Each subpixel 10 includes a reflective electrode serving as a first electrode 2, an insulating layer 3 covering the edge of the first electrode 2, an organic compound layer 4 covering the first electrode 2 and the insulating layer 3, a transparent electrode serving as a second electrode 5, a protective layer 6, and a color filter 7 over an interlayer insulating layer 1. Reference numeral 8 denotes the organic light-emitting device.

The transistors and capacitive elements may be disposed under or in the interlayer insulating layer 1. Each transistor may be electrically coupled to a corresponding one of the first electrodes 2 through, for example, a contact hole (not illustrated).

The insulating layer 3 is also referred to as a bank or pixel separation film. The insulating layer covers the edge of each first electrode 2 and surrounds the first electrode 2. Portions that are not covered with the insulating layer 3 are in contact with the organic compound layer 4 and serve as light-emitting regions.

The second electrode 5 may be a transparent electrode, a reflective electrode, or a semi-transparent electrode.

The protective layer 6 reduces the penetration of moisture into the organic compound layer 4. Although the protective layer 6 is illustrated as a single layer, the protective layer 6 may be formed of multiple layers. Each layer may be an inorganic compound layer or an organic compound layer.

The color filter 7 is separated into 7R, 7G, and 7B according to its color. The color filter 7 may be disposed on a planarization layer (not illustrated). A resin protective layer, which is not illustrated, may be disposed on the color filter 7. The color filter 7 may also be formed on the protective layer 6. Alternatively, the color filter may be disposed on an opposite substrate, such as a glass substrate, and then bonded.

FIG. 1B is a schematic cross-sectional view illustrating an example of a display apparatus including organic light-emitting devices and transistors coupled to the organic light-emitting devices. The organic light-emitting devices 26 and thin-film transistors (TFTs) 18 as an example of transistors are provided. A substrate 11 composed of, for example, glass or silicon, is provided, and an insulating layer 12 is disposed thereon. Active elements, such as the TFTs 18, are disposed on the insulating layer 12. The gate electrode 13, the gate insulating film 14, and the semiconductor layer 15 of each of the active elements are disposed thereon. Each TFT 18 further includes a drain electrode 16 and a source electrode 17. The TFTs 18 are overlaid with an insulating film 19. Anodes 21 included in the organic light-emitting devices 26 are coupled to the source electrodes 17 through contact holes 20 provided in the insulating film 19.

The mode of electrical connection between the electrodes (anode 21 and cathode 23) included in each organic light-emitting device 26 and the electrodes (source electrode 17 and drain electrode 16) included in a corresponding one of the TFTs 18 is not limited to the mode illustrated in FIG. 1B. That is, it is sufficient that any one of the anode 21 and the cathode 23 be electrically coupled to any one of the source electrode 17 and the drain electrode 16 of the TFT 18.

In the display apparatus illustrated in FIG. 1B, each organic compound layer 22 is illustrated as a single layer; however, the organic compound layer 22 may be formed of multiple layers. A first protective layer 24 and a second protective layer 25 are disposed on the cathodes 23 in order to reduce the deterioration of the organic light-emitting devices 26.

The transistors used in the display apparatus illustrated in FIG. 1B are not limited to transistors using a single-crystal silicon wafer, but may also be thin-film transistors including active layers on the insulating surface of a substrate. Examples of the active layer include single-crystal silicon, non-single-crystal silicon materials, such as amorphous silicon and microcrystalline silicon, and non-single-crystal oxide semiconductors, such as indium-zinc oxide and indium-gallium-zinc oxide.

The transistors in the display apparatus illustrated in FIG. 1B may be formed in the substrate, such as a Si substrate. The expression “formed in the substrate” indicates that the transistors are produced by processing the substrate, such as a Si substrate. When the transistors are formed in the substrate, the substrate and the transistors can be deemed to be integrally formed.

In the organic light-emitting devices 26 according to the present embodiment, the luminance is controlled by the TFTs, which are an example of switching elements; thus, an image can be displayed at respective luminance levels by arranging multiple organic light-emitting devices 26 in the plane. The switching elements according to the present embodiment are not limited to the TFTs and may be low-temperature polysilicon transistors or active-matrix drivers formed on a substrate such as a Si substrate. The expression “on a substrate” can also be said to be “in the substrate”. Whether transistors are formed in the substrate or TFT elements are used is selected in accordance with the size of a display unit. For example, when the display unit has a size of about 0.5 inches, organic light-emitting devices are preferably disposed on a Si substrate.

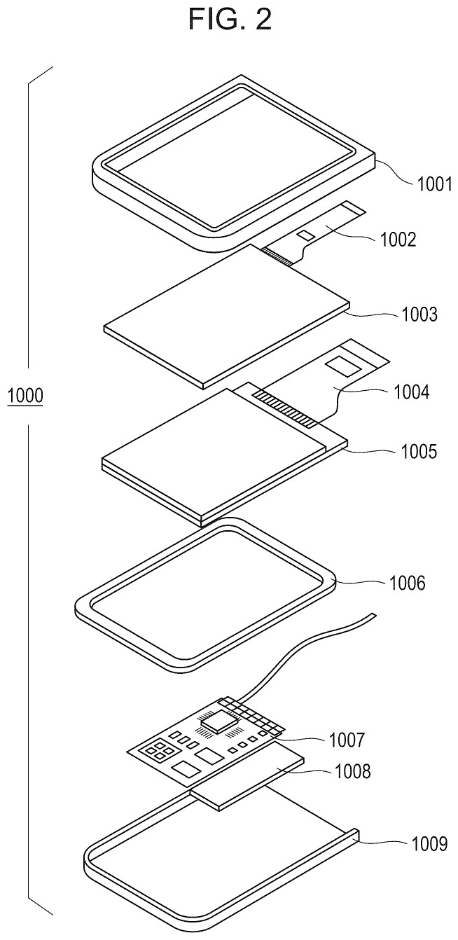

FIG. 2 is a schematic view illustrating an example of a display apparatus according to the present embodiment. A display apparatus 1000 includes, between an upper cover 1001 and a lower cover 1009, a touch panel 1003, a display panel 1005, a frame 1006, a circuit substrate 1007, and a battery 1008. The touch panel 1003 and the display panel 1005 are coupled to flexible printed circuits FPCs 1002 and 1004, respectively. The circuit substrate 1007 includes printed transistors. The battery 1008 need not be provided unless the display apparatus is a portable apparatus. The battery 1008 may be disposed at a different position even if the display apparatus is a portable apparatus.

The display apparatus according to the present embodiment may include a color filter having red, green, and blue portions. In the color filter, the red, green, and blue portions may be arranged in a delta arrangement.

The display apparatus according to the present embodiment may be used for the display unit of a portable terminal. In that case, the display apparatus may have both a display function and an operation function. Examples of the portable terminal include mobile phones, such as smartphones, tablets, and head-mounted displays.

The display apparatus according to the present embodiment may be used for a display unit of an image pickup apparatus including an optical unit including multiple lenses and an image pickup device that receives light passing through the optical unit. The image pickup apparatus may include a display unit that displays information acquired by the image pickup device. The display unit may be a display unit exposed to the outside of the image pickup apparatus or may be a display unit disposed in a finder. The image pickup apparatus may be a digital camera or a digital camcorder.

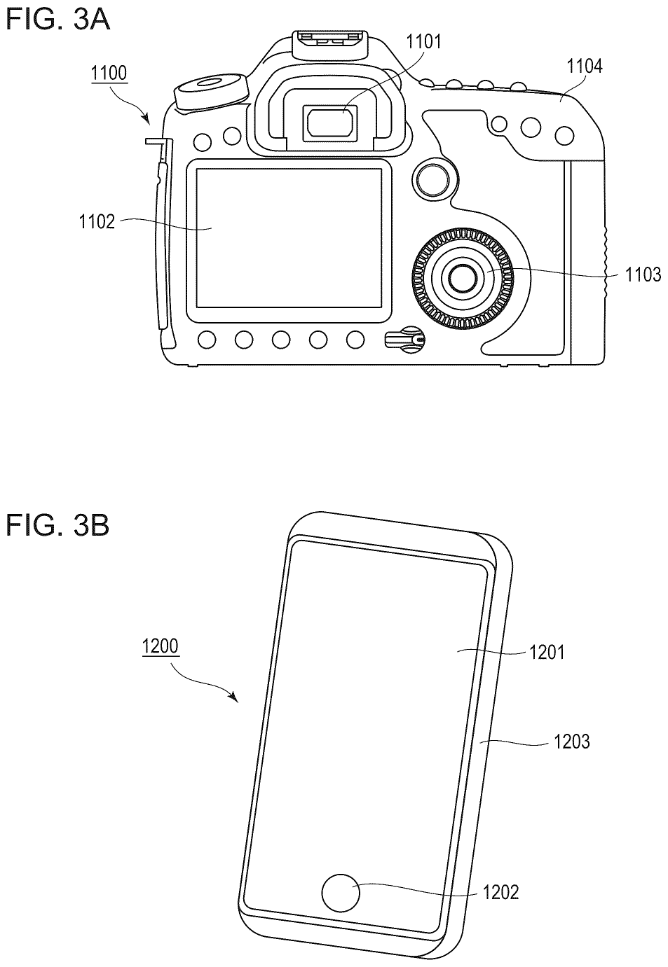

FIG. 3A is a schematic view illustrating an example of an image pickup apparatus according to the present embodiment. An image pickup apparatus 1100 may include a viewfinder 1101, a rear display 1102, an operation unit 1103, and a housing 1104. The viewfinder 1101 includes the display apparatus according to the present embodiment. In this case, the display apparatus may display environmental information, imaging instructions, and so forth in addition to an image to be captured. The environmental information may include, for example, the intensity of external light, the direction of the external light, the moving speed of a subject, and the possibility that a subject is shielded by a shielding material.

The timing suitable for imaging is only for a short time; thus, it is better to display the information as soon as possible. Thus, the display apparatus including the organic light-emitting device of the present embodiment is used. This is because organic light-emitting devices have a fast response time. The display apparatus including the organic light-emitting device can be used more suitably than liquid crystal display apparatuses for such apparatuses required to have a high display speed.

The image pickup apparatus 1100 includes an optical unit, which is not illustrated. The optical unit includes multiple lenses and is configured to form an image on an image pickup device in the housing 1104. The relative positions of the multiple lenses can be adjusted to adjust the focal point. This operation can also be performed automatically. The image pickup apparatus can also be referred to as a photoelectric conversion apparatus. Examples of an image capturing method employed in the photoelectric conversion apparatus can include a method for detecting a difference from the previous image and a method for cutting out an image from images always recorded, instead of sequentially capturing images.

FIG. 3B is a schematic view illustrating an example of an electronic apparatus according to the present embodiment. An electronic apparatus 1200 includes a display unit 1201, an operation unit 1202, and a housing 1203. The housing 1203 may accommodate a circuit, a printed circuit board including the circuit, a battery, and a communication unit. The operation unit 1202 may be a button or a touch-panel-type reactive unit. The operation unit 1202 unit may be a biometric recognition unit that recognizes a fingerprint to release the lock or the like. An electronic apparatus including a communication unit can also be referred to as a communication apparatus. The electronic apparatus 1200 may further have a camera function by being equipped with a lens and an image pickup device. An image captured by the camera function is displayed on the display unit 1201. Examples of the electronic apparatus 1200 include smartphones and laptop computers.

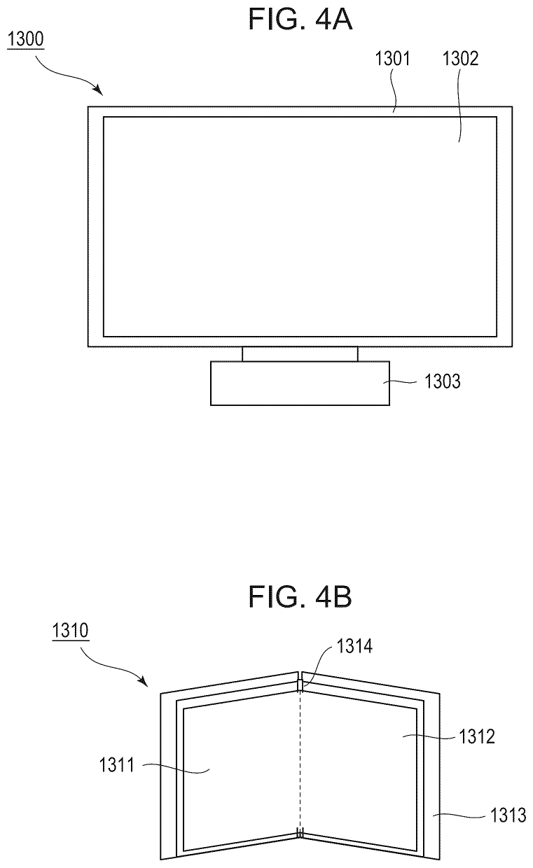

FIGS. 4A and 4B are each a schematic view illustrating an example of a display apparatus according to the present embodiment. FIG. 4A illustrates a display apparatus, such as a television monitor or a PC monitor. A display apparatus 1300 includes a frame 1301 and includes a display unit 1302. The organic light-emitting device according to the present embodiment is used for the display unit 1302. The frame 1301 and the display unit 1302 are supported by a base 1303. The base 1303 is not limited to a form illustrated in FIG. 4A. The lower side of the frame 1301 may also serve as a base. The frame 1301 and the display unit 1302 may be curved. The radius of curvature may be 5,000 mm or more and 6,000 mm or less.

FIG. 4B is a schematic view illustrating another example of a display apparatus according to the present embodiment. A display apparatus 1310 illustrated in FIG. 4B can be folded and is what is called a foldable display apparatus. The display apparatus 1310 includes a first display unit 1311, a second display unit 1312, a housing 1313, and an inflection point 1314. The first display unit 1311 and the second display unit 1312 include the organic light-emitting device according to the present embodiment. The first display unit 1311 and the second display unit 1312 may be a single, seamless display apparatus. The first display unit 1311 and the second display unit 1312 can be divided from each other at the inflection point. The first display unit 1311 and the second display unit 1312 may display different images from each other. Alternatively, a single image may be displayed in the first and second display units.

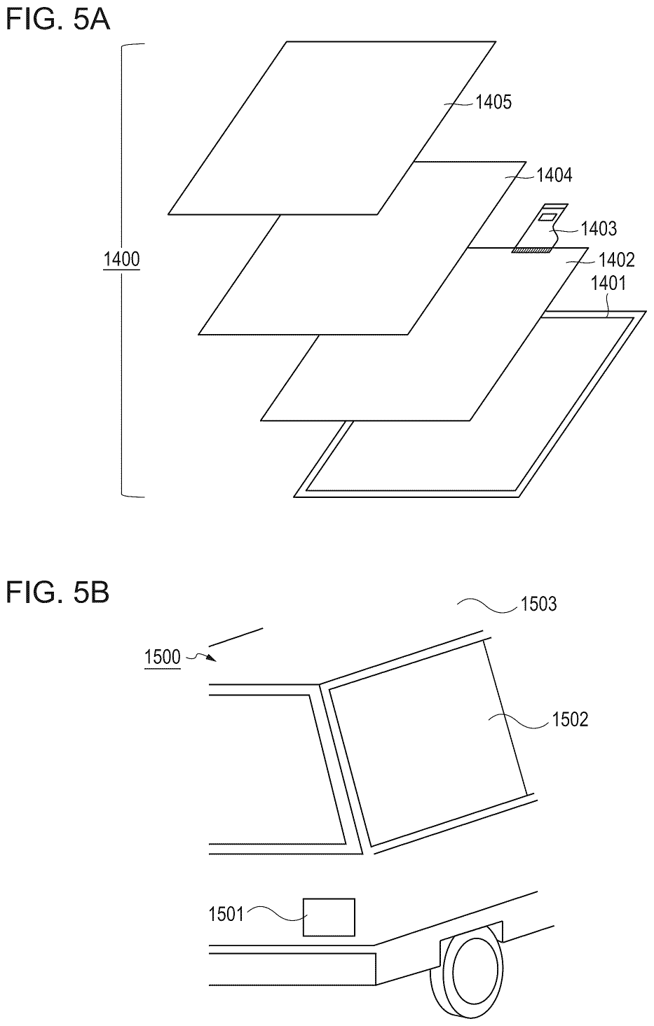

FIG. 5A is a schematic view illustrating an example of a lighting apparatus according to the present embodiment. A lighting apparatus 1400 may include a housing 1401, a light source 1402, a circuit board 1403, an optical filter 1404 that transmits light emitted from the light source 1402, and a light diffusion unit 1405. The light source 1402 includes an organic light-emitting device according to the present embodiment. The optical filter 1404 may be a filter that improves the color rendering properties of the light source. The light diffusion unit 1405 can effectively diffuse light from the light source to deliver the light to a wide range when used for illumination and so forth. The optical filter 1404 and the light diffusion unit 1405 may be disposed at the light emission side of the lighting apparatus. A cover may be disposed at the outermost portion, as needed.

The lighting apparatus is, for example, an apparatus that lights a room. The lighting apparatus may emit light of white, neutral white, or any color from blue to red. A light control circuit that controls the light may be provided. The lighting apparatus may include the organic light-emitting device according to the present embodiment and a power supply circuit coupled to the organic light-emitting device. The power supply circuit is a circuit that converts an AC voltage into a DC voltage. The color temperature of white is 4,200 K, and the color temperature of neutral white is 5,000 K. The lighting apparatus may include a color filter.

The lighting apparatus according to the present embodiment may include a heat dissipation unit. The heat dissipation unit is configured to release heat in the apparatus to the outside of the apparatus and is composed of, for example, a metal having a high specific heat or liquid silicone.

FIG. 5B is a schematic view of an automobile as an example of a moving object according to the present embodiment. The automobile includes a tail lamp, which is an example of lighting units. An automobile 1500 includes a tail lamp 1501 and may be configured to light the tail lamp when an operation, such as breaking, is performed.

The tail lamp 1501 includes an organic light-emitting device according to the present embodiment. The tail lamp 1501 may include a protective member that protects the organic light-emitting device. The protective member may be composed of any material as long as it has a certain degree of strength and is transparent. The protective member is preferably composed of, for example, polycarbonate. The polycarbonate may be mixed with, for example, a furandicarboxylic acid derivative or an acrylonitrile derivative.

The automobile 1500 may include an automobile body 1503 and windows 1502 attached thereto. The windows 1502 may be transparent displays unless they are windows used to check areas in front of and behind the automobile. The transparent displays may include the organic light-emitting devices according to the present embodiment. In this case, the constituent materials, such as the electrodes, of the organic light-emitting devices are formed of transparent members.

The moving object according to the present embodiment may be, for example, a ship, an aircraft, or a drone. The moving object may include a body and a lighting unit attached to the body. The lighting unit may emit light to indicate the position of the body. The lighting unit includes the organic light-emitting device according to the present embodiment.

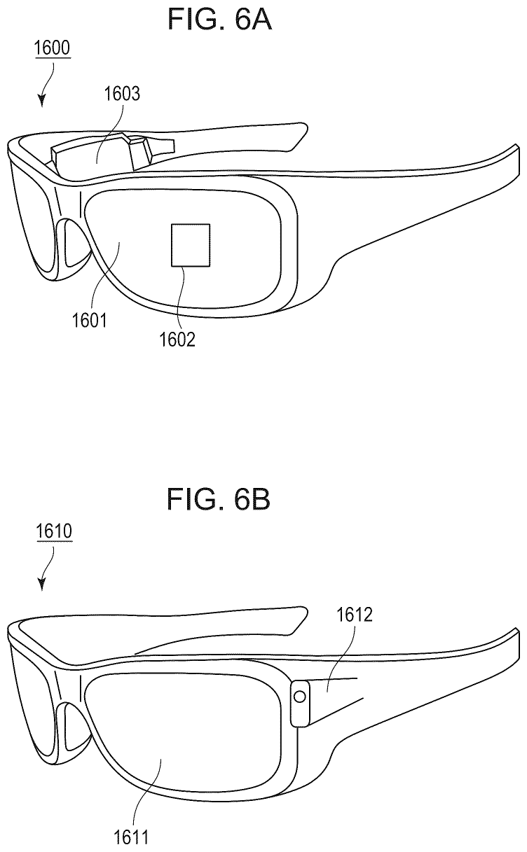

Examples of applications of the display apparatus of the above embodiment will be described with reference to FIGS. 6A and 6B. The display apparatus can be used for systems that can be worn as wearable devices, such as smart glasses, HMDs, and smart contact lenses. An image pickup and display apparatus used in such an application includes an image pickup apparatus that can photoelectrically convert visible light and a display apparatus that can emit visible light.

FIG. 6A is a schematic view illustrating an example of a wearable device according to an embodiment of the present invention. Glasses 1600 (smart glasses) according to an example of applications will be described with reference to FIG. 6A. An image pickup apparatus 1602, such as a CMOS sensor or SPAD, is provided on a front side of a lens 1601 of the glasses 1600. The display apparatus according to any of the above-mentioned embodiments is provided on the back side of the lens 1601.

The glasses 1600 further include a control unit 1603. The control unit 1603 functions as a power supply that supplies electric power to the image pickup apparatus 1602 and the display apparatus. The control unit 1603 controls the operation of the image pickup apparatus 1602 and the display apparatus. The lens 1601 includes an optical system for focusing light on the image pickup apparatus 1602.

FIG. 6B is a schematic view illustrating another example of a wearable device according to an embodiment of the present invention. Glasses 1610 (smart glasses) according to an example of applications will be described with reference to FIG. 6B. The glasses 1610 include a control unit 1612. The control unit 1612 includes an image pickup apparatus corresponding to the image pickup apparatus 1602 illustrated in FIG. 6A and a display apparatus. A lens 1611 is provided with the image pickup apparatus in the control unit 1612 and an optical system that projects light emitted from the display apparatus. An image is projected onto the lens 1611. The control unit 1612 functions as a power source that supplies electric power to the image pickup apparatus and the display apparatus and controls the operation of the image pickup apparatus and the display apparatus.

The control unit 1612 may include a gaze detection unit that detects the gaze of a wearer. Infrared radiation may be used for gaze detection. An infrared light-emitting unit emits infrared light to an eyeball of a user who is gazing at a displayed image. An image of the eyeball is captured by detecting the reflected infrared light from the eyeball with an image pickup unit having light-receiving elements. A deterioration in image quality is reduced by providing a reduction unit that reduces light from the infrared light-emitting unit to the display unit when viewed in plan. The user's gaze at the displayed image is detected from the image of the eyeball captured with the infrared light. Any known method can be used to the gaze detection using the captured image of the eyeball. As an example, a gaze detection method based on a Purkinje image of the reflection of irradiation light on a cornea can be used. More specifically, the gaze detection process is performed on the basis of a pupil-corneal reflection method. Using the pupil-corneal reflection method, the user's gaze is detected by calculating a gaze vector representing the direction (rotation angle) of the eyeball based on the image of the pupil and the Purkinje image contained in the captured image of the eyeball.

A display apparatus according to an embodiment of the present invention may include an image pickup apparatus including light-receiving elements, and may control an image displayed on the display apparatus based on the gaze information of the user from the image pickup apparatus. Specifically, in the display apparatus, a first field-of-view area at which the user gazes and a second field-of-view area other than the first field-of-view area are determined on the basis of the gaze information. The first field-of-view area and the second field-of-view area may be determined by the control unit of the display apparatus or may be determined by receiving those determined by an external control unit. In the display area of the display apparatus, the display resolution of the first field-of-view area may be controlled to be higher than the display resolution of the second field-of-view area. That is, the resolution of the second field-of-view area may be lower than that of the first field-of-view area.

The display area includes a first display area and a second display area different from the first display area. Based on the gaze information, an area of higher priority is determined from the first display area and the second display area. The first display area and the second display area may be determined by the control unit of the display apparatus or may be determined by receiving those determined by an external control unit. The resolution of an area of higher priority may be controlled to be higher than the resolution of an area other than the area of higher priority. In other words, the resolution of an area of a relatively low priority may be low.

Artificial intelligence (AI) may be used to determine the first field-of-view area and the high-priority area. The AI may be a model configured to estimate the angle of gaze from the image of the eyeball and the distance to a target object located in the gaze direction, using the image of the eyeball and the actual direction of gaze of the eyeball in the image as teaching data. The AI program may be stored in the display apparatus, the image pickup apparatus, or an external apparatus. When the AI program is stored in the external apparatus, the AI program is transmitted to the display apparatus via communications.

In the case of controlling the display based on visual detection, smart glasses that further include an image pickup apparatus that captures an external image can preferably be used. The smart glasses can display the captured external information in real time.

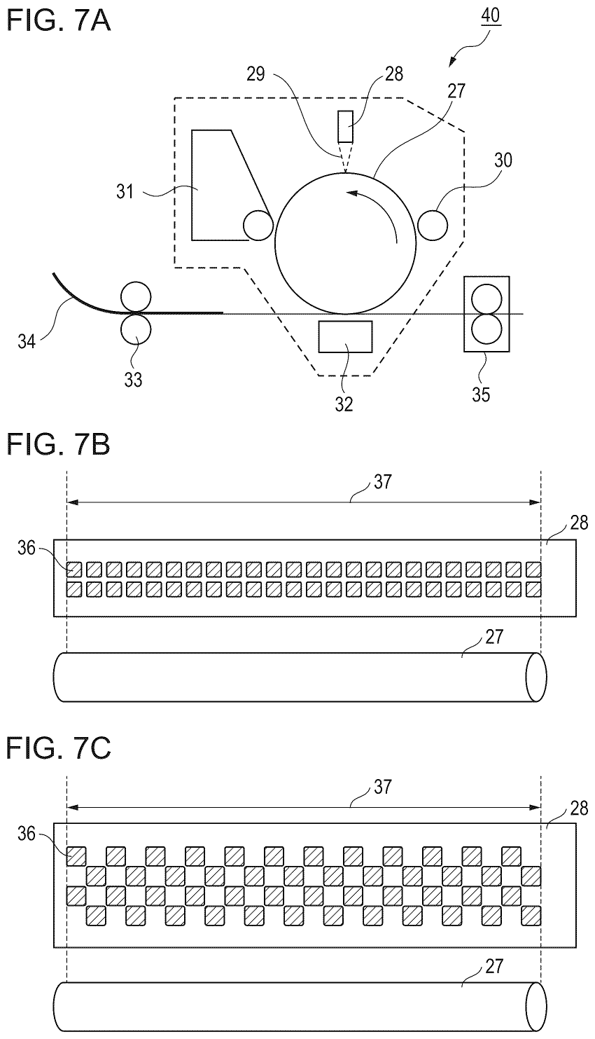

FIG. 7A is a schematic view illustrating an example of an image-forming apparatus according to an embodiment of the present invention. An image-forming apparatus 40 is an electrophotographic image-forming apparatus and includes a photoconductor 27, an exposure light source 28, a charging unit 30, a developing unit 31, a transfer unit 32, a conveying roller 33, and a fixing unit 35. The irradiation of light 29 is performed from the exposure light source 28 to form an electrostatic latent image on the surface of the photoconductor 27. The exposure light source 28 includes the organic light-emitting device according to the present embodiment. The developing unit 31 contains, for example, a toner. The charging unit 30 charges the photoconductor 27. The transfer unit 32 transfers the developed image to a recording medium 34. The conveying roller 33 conveys the recording medium 34. The recording medium 34 is paper, for example. The fixing unit 35 fixes the image formed on the recording medium 34.

FIGS. 7B and 7C each illustrate the exposure light source 28 and are each a schematic view illustrating multiple light-emitting portions 36 arranged on a long substrate. Arrows 37 are parallel to the axis of the photoconductor and each represent the row direction in which the light-emitting portions 36 including the organic light-emitting devices are arranged. The row direction is the same as the direction of the axis about which the photoconductor 27 rotates. This direction can also be referred to as the long-axis direction of the photoconductor 27. FIG. 7B illustrates a configuration in which the light-emitting portions 36 are arranged in the long-axis direction of the photoconductor 27. FIG. 7C is different from FIG. 7B in that the light-emitting portions 36 are arranged alternately in the row direction in a first row and a second row. The first row and the second row are located at different positions in the column direction. In the first row, the multiple light-emitting portions 36 are spaced apart. The second row has the light-emitting portions 36 at positions corresponding to the positions between the light-emitting portions 36 in the first row. In other words, the multiple light-emitting portions 36 are also spaced apart in the column direction. The arrangement in FIG. 7C can be rephrased as, for example, a lattice arrangement, a staggered arrangement, or a checkered pattern.

As described above, the use of an apparatus including the organic light-emitting device according to the present embodiment enables a stable display with good image quality even for a long time.

### Included Configuration

The disclosure of the present embodiment includes the following configurations.

### Configuration 1

An organic light-emitting device includes:
a first electrode, a light-emitting layer, and a second electrode,
in which the light-emitting layer contains a first organic compound, a second organic compound, and a third organic compound,
a freely rotatable single bond in each of the first organic compound, the second organic compound, and the third organic compound is a carbon-carbon bond,
at least one carbon atom of a freely rotatable carbon-carbon bond contained in the second organic compound is an sp² carbon atom, and
letting the HOMO energy level of the first organic compound be HOMO1, letting the LUMO energy level of the second organic compound be LUMO2, and letting HOMO and LUMO energy levels of the third organic compound be HOMO3 and LUMO3, [I] and [II] are satisfied:

|LUMO2|>|LUMO3| [I]

|HOMO3|>|HOMO1| [II].

### Configuration 2

In the organic light-emitting device described in configuration 1, carbon atoms of the freely rotatable carbon-carbon bond of the second organic compound are each an sp² carbon atom.

### Configuration 3

In the organic light-emitting device described in configuration 1 or 2, carbon atoms of freely rotatable carbon-carbon bonds contained in the first organic compound and the third organic compound are each an sp² carbon atom.

### Configuration 4

In the organic light-emitting device described in any one of configurations 1 to 3, amounts of the first to third organic compounds contained in the light-emitting layer satisfy the following relationship: the third organic compound>the first organic compound≥the second organic compound.

### Configuration 5

In the organic light-emitting device described in any one of configurations 1 to 4, the first organic compound, the second organic compound, and the third organic compound each contain no alkyl group.

### Configuration 6

In the organic light-emitting device described in any one of configurations 1 to 5, the second organic compound is a blue-light-emitting material.

### Configuration 7

In the organic light-emitting device described in any one of configurations 1 to 6, the second organic compound contains a fluoranthene skeleton.

### Configuration 8

In the organic light-emitting device described in any one of configurations 1 to 7, the third organic compound contains any one of an anthracene skeleton, a phenanthroline skeleton, a pyrene skeleton, a chrysene skeleton, a benzophenanthrene skeleton, a triphenylene skeleton, a fluoranthene skeleton, a benzochrysene skeleton, and a benzofluoran skeleton.

### Configuration 9

In the organic light-emitting device described in any one of configurations 1 to 8, the third organic compound contains a pyrene skeleton.

### Configuration 10

In the organic light-emitting device described in any one of configurations 1 to 9, the first organic compound is a compound represented by the following general formula [1]

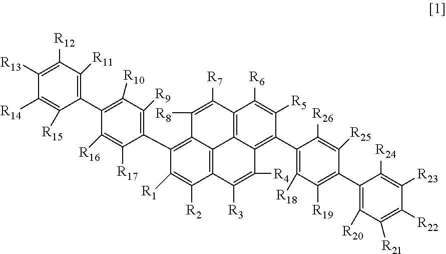

where in general formula [1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, R₁₀ and R₁₁, R₁₅ and R₁₆, R₁₉ and R₂₀, and R₂₄ and R₂₅ may form respective rings, each ring containing a carbon atom, an oxygen atom, a sulfur atom, a selenium atom, or a tellurium atom serving as a spacer, and R₁₁ to R₁₅ and R₂₀ to R₂₄ may each form a ring with an adjacent substituent].

### Configuration 11

In the organic light-emitting device described in any one of configurations 1 to 9, the first organic compound is a compound represented by the following general formula [2]:

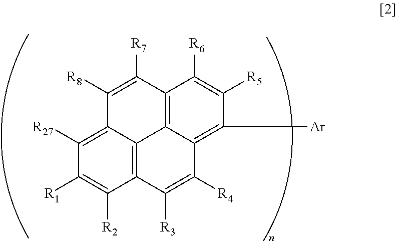

where in general formula [2], R₁ to R₈ and R₂₇ are each independently selected from a hydrogen atom, a deuterium atom, a substituted or unsubstituted alkyl group, a substituted or unsubstituted aryl group, and a substituted or unsubstituted heterocyclic group, and n is a natural number selected from 1 to 3.

### Configuration 12

In the organic light-emitting device described in any one of configurations 1 to 9, the first organic compound is a compound represented by the following general formula [1-1] or [1-2]:

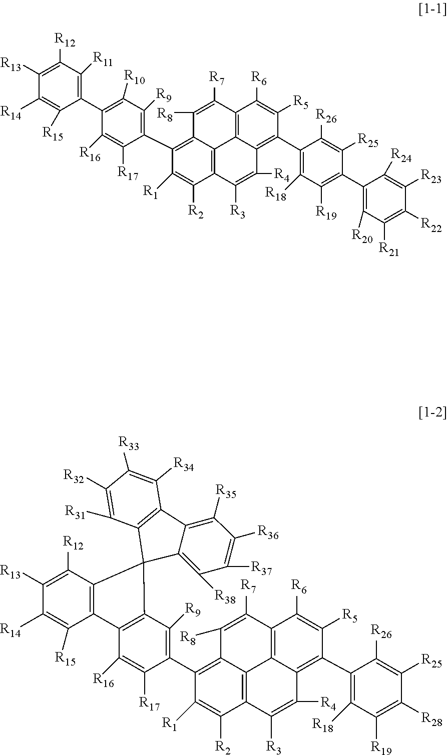

where in general formula [1-1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₀ and R₁₁, R₁₅ and R₁₆, R₁₉ and R₂₀, and R₂₄ and R₂₅ forms a ring with a carbon atom, an oxygen atom, a sulfur atom, a selenium atom, or a tellurium atom serving as a spacer, and R₁₁ to R₁₅ and R₂₀ to R₂₄ may each form a ring with an adjacent substituent, and
where in general formula [1-2], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, and R₁₈ and R₁₉, R₂₅ and R₂₆, and R₂₈ may each form a ring with an adjacent substituent].

### Configuration 13

In the organic light-emitting device described in configuration 12, the first organic compound is a compound represented by the following general formula [1-3]:

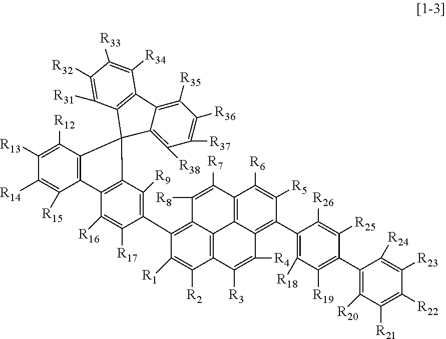

where in general formula [1-3], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₉ and R₂₀, and R₂₄ and R₂₅ forms a ring with a carbon atom, an oxygen atom, a sulfur atom, a selenium atom, or a tellurium atom serving as a spacer, and R₂₀ to R₂₄ each optionally form a ring with an adjacent substituent.

### Configuration 14

In the organic light-emitting device described in any one of configurations 1 to 9, the first organic compound is a compound containing a skeleton represented by any one of the following general formulae [3-1] to [3-4]:

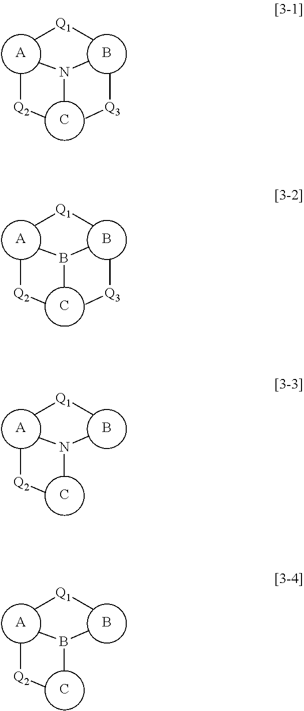

where in each of general formulae [3-1] to [3-4], cyclic units A to C are each independently selected from an aromatic hydrocarbon group and a heterocyclic group and may each be a fused ring, Q₁ to Q₃ are each independently selected from a direct bond, CR₁R₂, NR₃, an oxygen atom, a sulfur atom, a selenium atom, and a tellurium atom, substituents R₁ to R₃ are each independently selected from a substituted or unsubstituted alkyl group, a substituted or unsubstituted alkoxy group, a substituted or unsubstituted aryl group, and a substituted or unsubstituted heteroaryl group, and R₃ may form a ring together with any one of adjacent cyclic units A to C.

### Configuration 15

The organic light-emitting device described in any one of configurations 1 to 14 further includes a hole-blocking layer in contact with the light-emitting layer, in which the hole-blocking layer contains a compound with a fused-ring skeleton composed of a hydrocarbon.

### Configuration 16

The organic light-emitting device described in any one of configurations 1 to 15 further includes an electron-blocking layer in contact with the light-emitting layer, in which a freely rotatable single bond in a compound contained in the electron-blocking layer is a carbon-carbon bond.

### Configuration 17

The organic light-emitting device described in any one of configurations 1 to 16 further includes a second light-emitting layer disposed in contact with the first light-emitting layer, in which the second light-emitting layer has an emission color different from the emission color of the first light-emitting layer.

### Configuration 18

A display apparatus includes multiple pixels, at least one of the multiple pixels including the organic light-emitting device described in any one of configurations 1 to 17 and a transistor coupled to the organic light-emitting device.

### Configuration 19

A photoelectric conversion apparatus includes an optical unit including multiple lenses, an image pickup device configured to receive light passing through the optical unit, and a display unit configured to display an image captured by the image pickup device, in which the display unit includes the organic light-emitting device described in any one of configurations 1 to 17.

### Configuration 20

An electronic apparatus includes a display unit including the organic light-emitting device described in any one of configurations 1 to 17, a housing provided with the display unit, and a communication unit disposed in the housing and configured to communicate with the outside.

### Configuration 21

A lighting apparatus includes a light source including the organic light-emitting device described in any one of configurations 1 to 17, and a light diffusion unit or an optical filter configured to transmit light emitted from the light source.

### Configuration 22

A moving object includes a lighting unit including the organic light-emitting device described in any one of configurations 1 to 17, and a body provided with the lighting unit.

### Configuration 23

An exposure light source for an electrophotographic image-forming apparatus includes the organic light-emitting device described in any one of configurations 1 to 17.

### Configuration 24

An organic compound represented by the following general formula [1-1] or [1-2]:

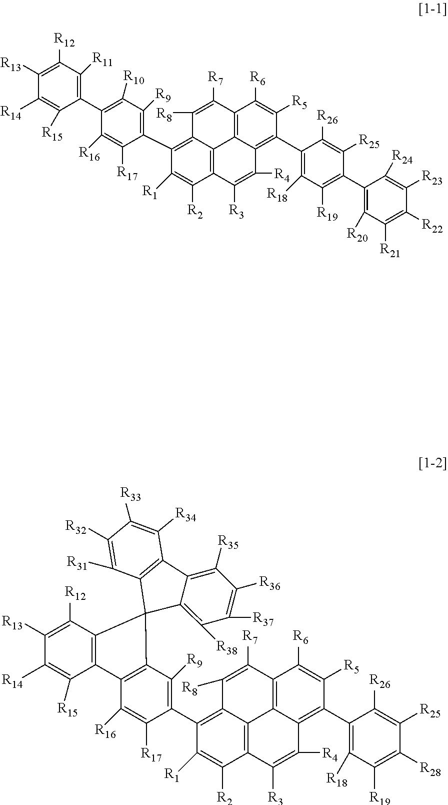

where in general formula [1-1] described above, R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₀ and R₁₁, R₁₅ and R₁₆, R₁₉ and R₂₀, and R₂₄ and R₂₅ forms a ring with a carbon atom, an oxygen atom, a sulfur atom, a selenium atom, or a tellurium atom serving as a spacer, and R₁₁ to R₁₅ and R₂₀ to R₂₄ each optionally form a ring with an adjacent substituent, and
where in general formula [1-2] described above, R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, and R₁₈ and R₁₉, R₂₅ and R₂₆, and R₂₈ may each form a ring with an adjacent substituent.

### Configuration 25

In the organic compound described in configuration 24, the organic compound is represented by the following general formula [1-3]:

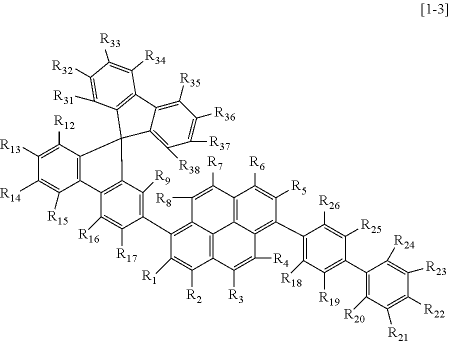

where in general formula [1-3], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₉ and R₂₀, and R₂₄ and R₂₅ forms a ring with a carbon atom, an oxygen atom, a sulfur atom, a selenium atom, or a tellurium atom serving as a spacer, and R₂₀ to R₂₄ each optionally form a ring with an adjacent substituent.

### EXAMPLES

The organic compounds used in Examples are illustrated below.

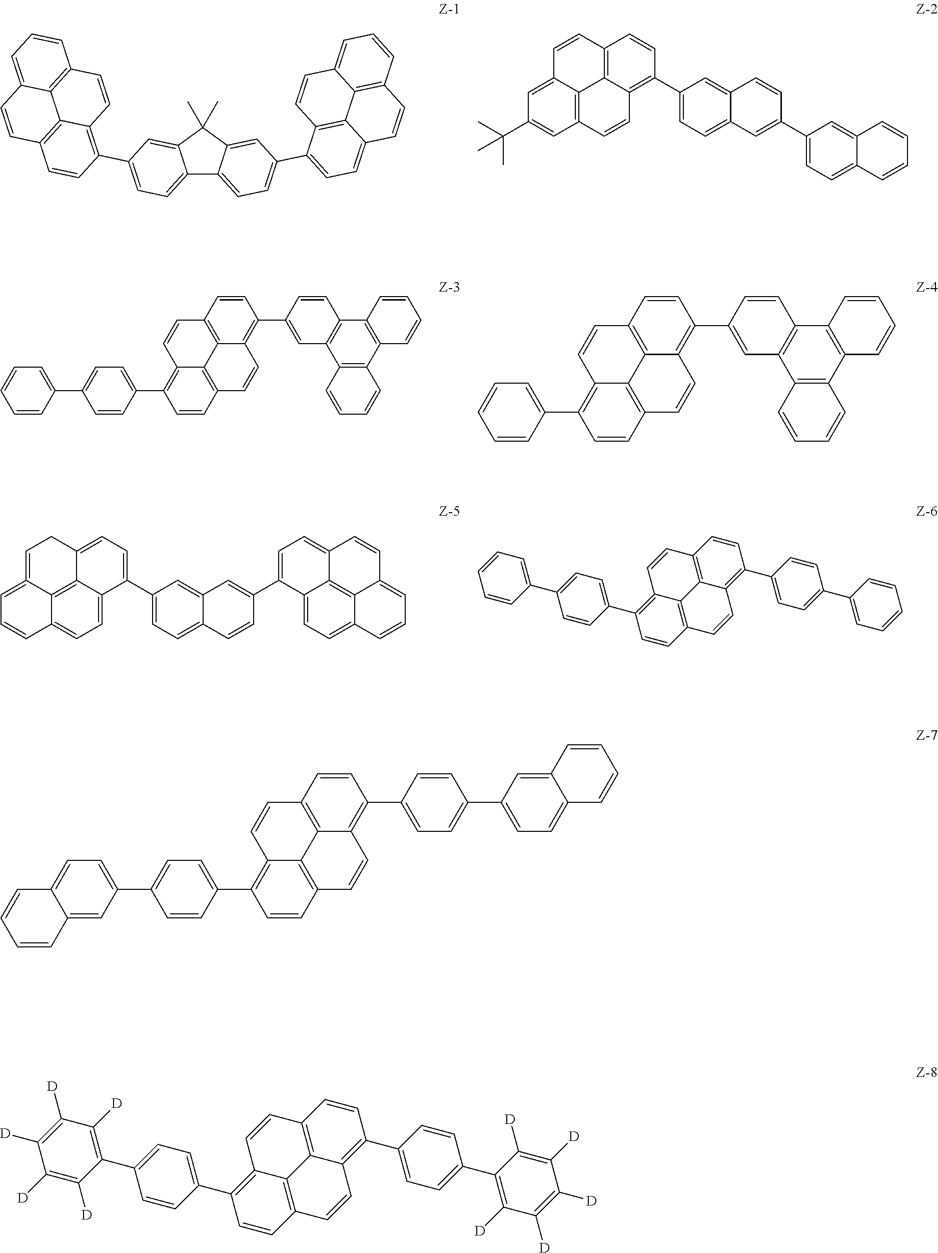

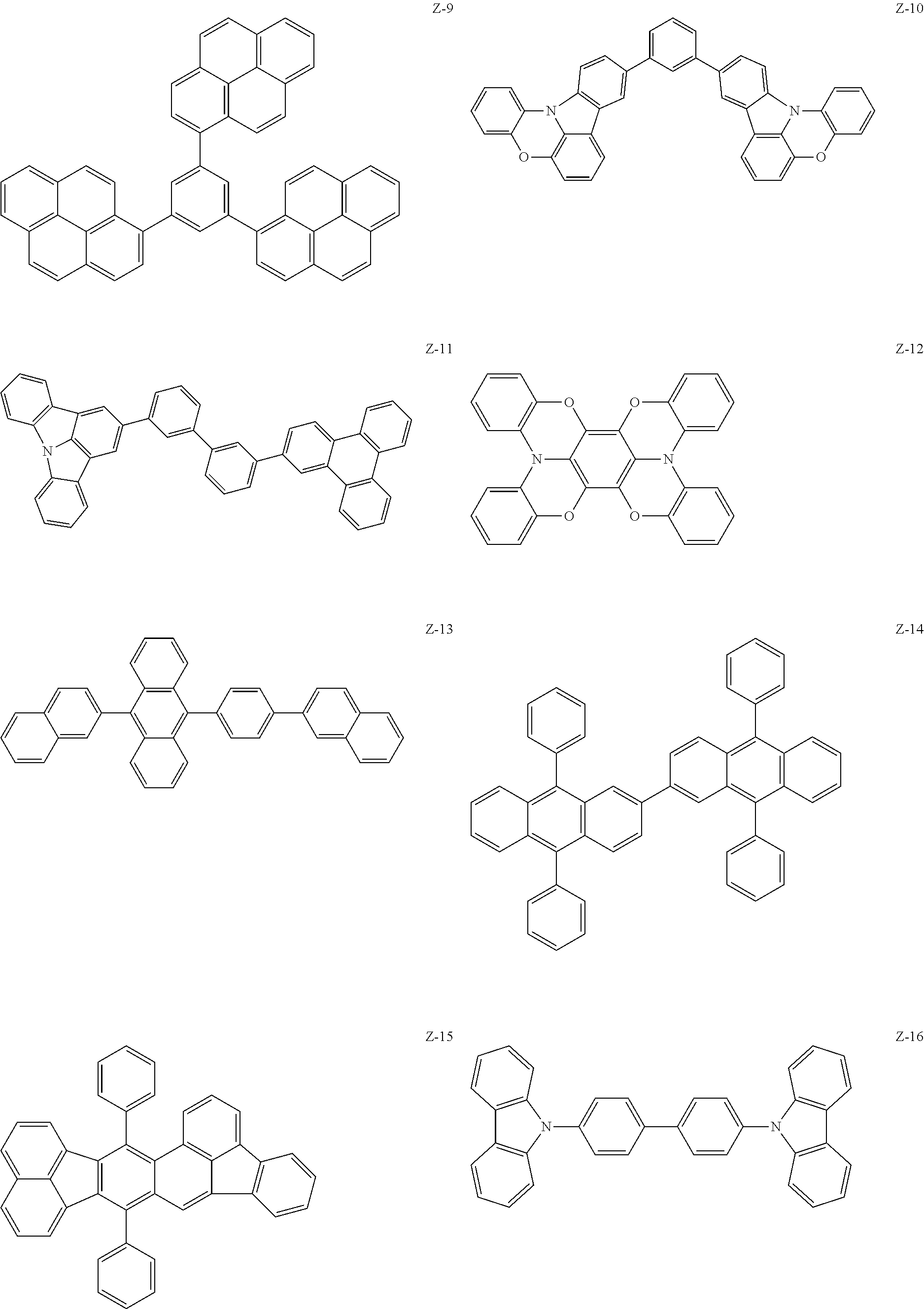

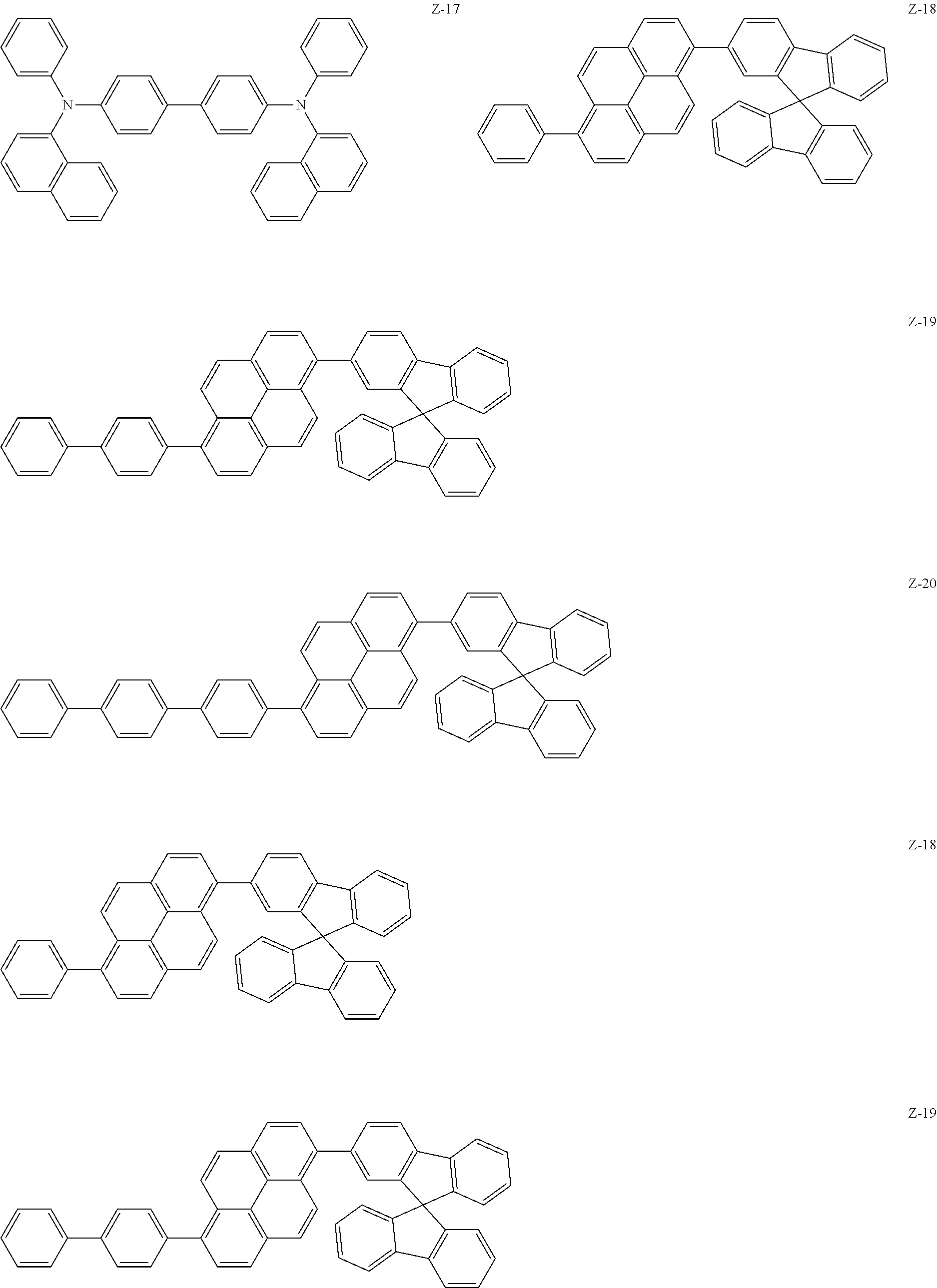

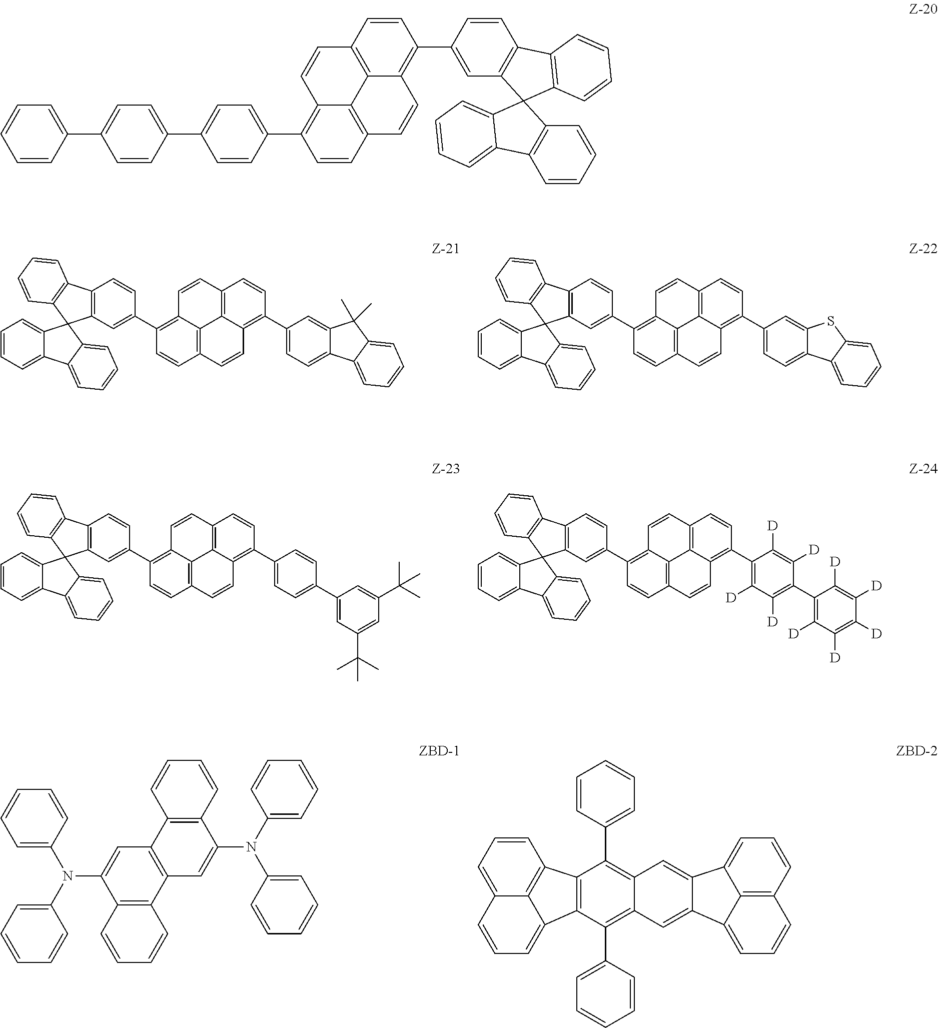

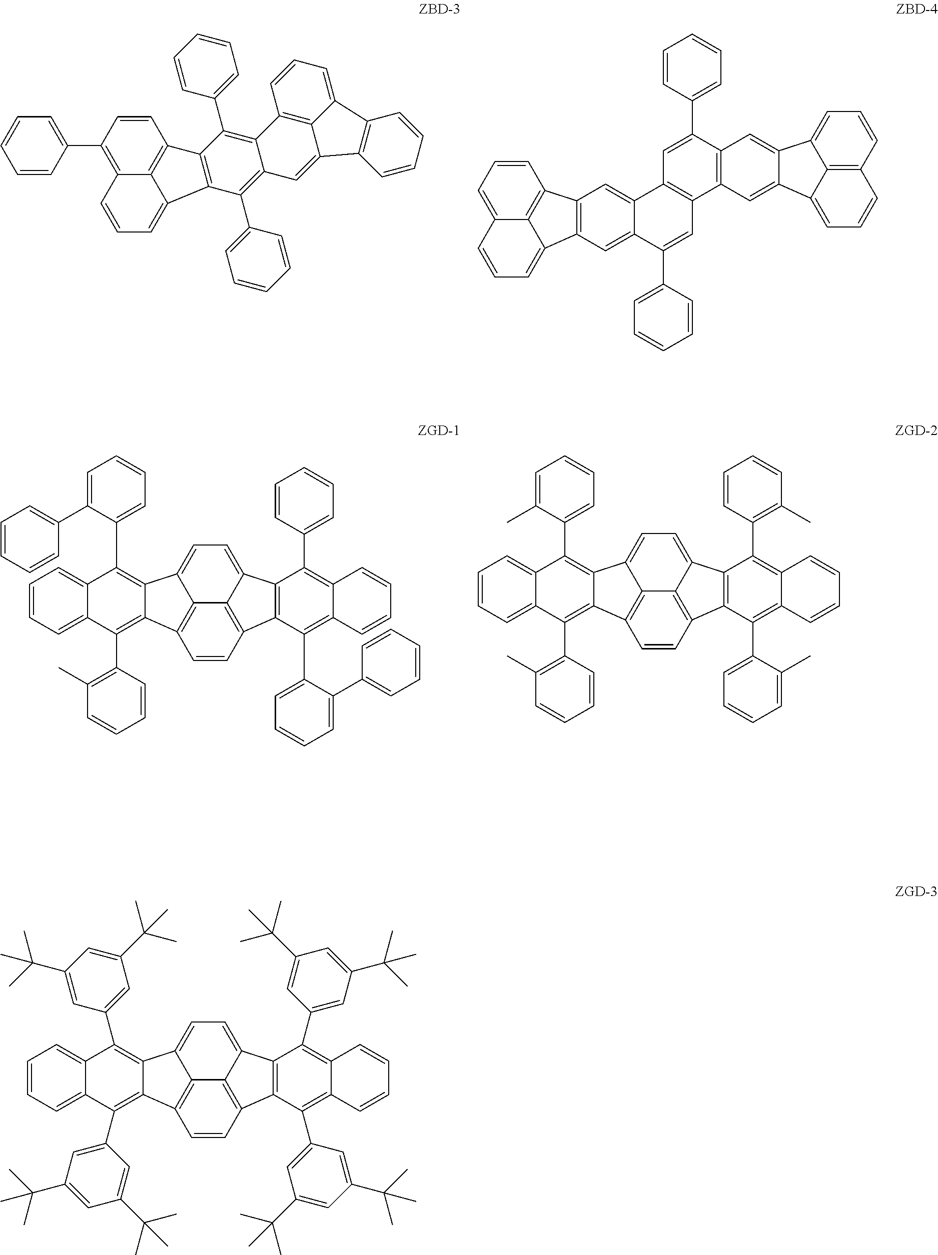

Table 7 presents the HOMO levels and LUMO levels of the organic compounds used in Examples. Each of the HOMO levels is the value of the ionization potential determined by forming a 50-nm film of a corresponding one of the organic compounds using a vacuum evaporation method and then measuring the resulting thin film with “AC-3” available from Riken Keiki Co., Ltd. Each LUMO level is a value obtained by measuring the absorption spectrum of the thin film, determining the optical absorption edge as a band gap, and then subtracting this from the value of the ionization potential.

TABLE 7

| Organic | HOMO | LUMO |
|---|---|---|
| compound | [eV] | [eV] |
| Z-1 | −6.0 | −3.0 |
| Z-2 | −5.8 | −2.8 |
| Z-3 | −6.0 | −3.1 |
| Z-4 | −6.0 | −3.1 |
| Z-5 | −6.1 | −3.1 |
| Z-6 | −5.9 | −3.0 |
| Z-7 | −5.9 | −3.0 |
| Z-8 | −5.9 | −3.0 |
| Z-9 | −6.0 | −2.9 |
| Z-10 | −5.8 | −2.7 |
| Z-11 | −6.1 | −3.0 |
| Z-12 | −5.5 | −2.6 |
| Z-13 | −6.0 | −3.1 |
| Z-14 | −5.9 | −3.1 |
| Z-15 | −6.1 | −3.4 |
| Z-16 | −6.2 | −2.7 |
| Z-17 | −5.6 | −2.6 |
| Z-18 | −5.9 | −2.9 |
| Z-19 | −5.9 | −3.0 |
| Z-20 | −5.9 | −3.1 |
| Z-21 | −5.8 | −2.9 |
| Z-22 | −5.9 | −3.0 |
| Z-23 | −5.8 | −2.9 |
| Z-24 | −5.9 | −3.0 |
| ZBD-1 | −5.5 | −2.9 |
| ZBD-2 | −6.0 | −3.2 |
| ZBD-3 | −6.1 | −3.5 |
| ZBD-4 | −6.0 | −3.2 |
| ZGD-1 | −6.1 | −3.5 |
| ZGD-2 | −6.0 | −3.6 |
| ZGD-3 | −6.0 | −3.6 |

### Example 1

In this example, an organic light-emitting device having a top-emission structure was produced in which an anode, a hole injection layer, a hole transport layer, an electron-blocking layer, a light-emitting layer, a hole-blocking layer, an electron transport layer, an electron injection layer, and a cathode were sequentially formed over a substrate.

A Ti film having a thickness of 40 nm was formed by a sputtering method on a glass substrate and patterned using a photolithography, thereby forming the anode. The electrode area of the anode was set to 3 mm². The anode was then washed.

The substrate with the electrode formed above was attached to a vacuum evaporation apparatus (manufactured by ULVAC, Inc.). After preparation for evaporation of the evaporation material was made, the apparatus was evacuated to 1.33×10⁻⁴ Pa (1×10⁻⁶ Torr). The inside of the chamber was then subjected to UV/ozone cleaning. Thereafter, each layer was formed so as to achieve a layer configuration given in Table 8. The resulting substrate was transferred to a glove box and sealed with a glass cap containing a desiccant in a nitrogen atmosphere to provide an organic light-emitting device. The symbol “%” in the table indicates “% by mass”.

TABLE 8

| | Material | | | Thickness (nm) |
|---|---|---|---|---|
| Cathode | Al | | | 100 |
| Electron | LiF | | | 1 |
| injection layer | | | | |
| (EIL) | | | | |
| Electron | ET2 | | | 15 |
| transport layer | | | | |
| (ETL) | | | | |
| Hole-blocking | ET12 | | | 15 |
| layer | | | | |
| (HBL) | | | | |
| Light-emitting | Third | First | Second | 30 |
| layer | organic | organic | organic | |
| | compound | compound | compound | |
| | Z-1 | Z-2 | ZBD-2 | |
| | 84% | 15% | 1% | |
| Electron- | HT12 | | | 15 |
| blocking layer | | | | |
| (EBL) | | | | |
| Hole transport | HT3 | | | 30 |
| layer | | | | |
| (HTL) | | | | |
| Hole injection | HT16 | | | 5 |
| layer | | | | |
| (HIL) | | | | |

The resulting organic light-emitting device was connected to a voltage application device, and its characteristics were evaluated. The current-voltage characteristics were measured with a Hewlett-Packard microammeter 4140B. The chromaticity was evaluated with a Topcon “SR-3”. The luminance was measured with a Topcon BM7. When display was performed at a luminance of 1,000 cd/m², the external quantum efficiency (E.Q.E) was 5%, indicating a good organic blue-light-emitting device.

### Examples 2 to 26 and Comparative Examples 1 to 6

Organic light-emitting devices were produced and their characteristics were evaluated in the same manner as in Example 1, except that the configurations of the organic compound layers were changed as indicated in Tables 9 to 11. The symbol “%” in the tables indicates “% by mass”.

The device durability ratio was a ratio determined by performing a continuous operation test at an initial luminance of 5,000 cd/m², measuring the luminance after 100 hours, and setting the luminance of the device in Comparative Example 1 to 1.0.

TABLE 9

| | | | | EML | | | | | | |
|---|---|---|---|---|---|---|---|---|---|---|
| | | | | Third | First | Second | | | | Device |
| | | | | organic | organic | organic | | | Emission | durability |
| | HIL | HTL | EBL | compound | compound | compound | HBL | ETL | color | ratio |
| Example 1 | HT16 | HT3 | HT12 | Z-1 | Z-2 | ZBD-2 | ET12 | ET2 | blue | 1.3 |
| | | | | 84% | 15% | 1% | | | | |
| Comparative | HT16 | HT3 | HT12 | Z-1 | Z-17 | ZBD-2 | ET12 | ET2 | blue | 1.0 |
| Example 1 | | | | 84% | 15% | 1% | | | | |
| Comparative | HT16 | HT3 | HT12 | Z-1 | ZBD-1 | Z-13 | ET12 | ET2 | blue | 0.7 |
| Example 2 | | | | 84% | 1% | 15% | | | | |
| Comparative | HT16 | HT3 | HT12 | Z-16 | ZBD-1 | Z-13 | ET12 | ET2 | blue | 0.5 |
| Example 3 | | | | 84% | 1% | 15% | | | | |
| Example 2 | HT16 | HT3 | HT10 | Z-5 | Z-6 | ZGD-1 | ET13 | ET2 | green | 2.7 |
| | | | | 83% | 15% | 2% | | | | |
| Example 3 | HT16 | HT3 | HT10 | Z-1 | Z-14 | ZGD-1 | ET13 | ET2 | green | 1.7 |
| | | | | 83% | 15% | 2% | | | | |
| Example 4 | HT16 | HT3 | HT10 | Z-1 | Z-14 | ZGD-2 | ET13 | ET2 | green | 1.5 |
| | | | | 83% | 15% | 2% | | | | |
| Comparative | HT16 | HT3 | HT10 | Z-1 | Z-14 | ZGD-3 | ET13 | ET2 | green | 1.0 |
| Example 4 | | | | 83% | 15% | 2% | | | | |
| Example 5 | HT16 | HT3 | HT12 | Z-1 | Z-6 | ZBD-2 | ET12 | ET2 | blue | 2.0 |
| | | | | 84% | 15% | 1% | | | | |
| Example 6 | HT16 | HT3 | HT12 | Z-5 | Z-6 | ZBD-2 | ET12 | ET2 | blue | 2.6 |
| | | | | 84% | 15% | 1% | | | | |
| Example 7 | HT16 | HT3 | HT12 | Z-5 | Z-2 | ZBD-2 | ET12 | ET2 | blue | 1.7 |
| | | | | 84% | 15% | 1% | | | | |
| Example 8 | HT16 | HT3 | HT12 | Z-5 | Z-7 | ZBD-2 | ET12 | ET2 | blue | 2.6 |
| | | | | 84% | 15% | 1% | | | | |
| Example 9 | HT16 | HT3 | HT12 | Z-5 | Z-3 | ZBD-2 | ET12 | ET2 | blue | 2.4 |
| | | | | 84% | 15% | 1% | | | | |

TABLE 10

| | | | | EML | | | | | | |
|---|---|---|---|---|---|---|---|---|---|---|
| | | | | Third | First | Second | | | | Device |
| | | | | organic | organic | organic | | | Emission | durability |
| | HIL | HTL | EBL | compound | compound | compound | HBL | ETL | color | ratio |
| Example 10 | HT16 | HT3 | HT12 | Z-4 | Z-7 | ZBD-2 | ET12 | ET2 | blue | 2.1 |
| | | | | 84% | 15% | 1% | | | | |
| Example 11 | HT16 | HT3 | HT12 | Z-9 | Z-7 | ZBD-2 | ET12 | ET2 | blue | 2.1 |
| | | | | 84% | 15% | 1% | | | | |
| Example 12 | HT16 | HT3 | HT12 | Z-11 | Z-7 | ZBD-2 | ET12 | ET2 | blue | 2.3 |
| | | | | 84% | 15% | 1% | | | | |
| Comparative | HT16 | HT3 | HT12 | Z-13 | Z-7 | ZBD-2 | ET12 | ET2 | blue | 1.3 |
| Example 5 | | | | 84% | 15% | 1% | | | | |
| Comparative | HT16 | HT3 | HT12 | Z-16 | Z-7 | ZBD-2 | ET12 | ET2 | blue | 1.0 |
| Example 6 | | | | 84% | 15% | 1% | | | | |
| Example 13 | HT16 | HT3 | HT12 | Z-5 | Z-6 | ZBD-3 | ET12 | ET2 | blue | 2.9 |
| | | | | 84% | 15% | 1% | | | | |
| Example 14 | HT16 | HT3 | HT10 | Z-5 | Z-6 | ZBD-3 | ET12 | ET5 | blue | 2.9 |
| | | | | 84% | 15% | 1% | | | | |
| Example 15 | HT16 | HT3 | HT12 | Z-5 | Z-6 | ZBD-4 | ET12 | ET2 | blue | 2.6 |
| | | | | 84% | 15% | 1% | | | | |
| Example 16 | HT16 | HT3 | HT12 | Z-5 | Z-12 | ZBD-3 | ET12 | ET2 | blue | 2.9 |
| | | | | 98% | 1% | 1% | | | | |
| Example 17 | HT16 | HT3 | HT12 | Z-5 | Z-8 | ZBD-2 | ET12 | ET2 | blue | 2.8 |
| | | | | 84% | 15% | 1% | | | | |
| Example 18 | HT16 | HT3 | HT10 | Z-11 | Z-7 | ZBD-2 | ET12 | ET2 | blue | 2.3 |
| | | | | 84% | 15% | 1% | | | | |
| Example 19 | HT16 | HT3 | Z-10 | Z-11 | Z-7 | ZBD-2 | ET12 | ET2 | blue | 2.4 |
| | | | | 84% | 15% | 1% | | | | |
| Example 20 | HT16 | HT3 | HT10 | Z-11 | Z-7 | ZBD-2 | ET13 | ET2 | blue | 2.3 |
| | | | | 84% | 15% | 1% | | | | |
| Example 21 | HT16 | HT3 | HT10 | Z-11 | Z-7 | ZBD-2 | ET15 | ET2 | blue | 2.0 |
| | | | | 84% | 15% | 1% | | | | |
| Example 22 | HT16 | HT3 | HT12 | Z-9 | Z-7 | ZBD-2 | ET12 | ET2 | blue | 1.9 |
| | | | | 94% | 5% | 1% | | | | |
| Example 23 | HT16 | HT3 | HT12 | Z-9 | Z-7 | ZBD-2 | ET12 | ET2 | blue | 2.3 |
| | | | | 69% | 30% | 1% | | | | |

TABLE 11

| | | | | EML | | | | | | |
|---|---|---|---|---|---|---|---|---|---|---|
| | | | | Third | First | Second | | | | Device |
| | | | | organic | organic | organic | | | Emission | durability |
| | HIL | HTL | EBL | compound | compound | compound | HBL | ETL | color | ratio |
| Example 24 | HT16 | HT3 | HT12 | Z-5 | Z-19 | ZBD2 | ET12 | ET2 | blue | 2.7 |
| | | | | 84% | 15% | 1% | | | | |
| Example 25 | HT16 | HT3 | HT12 | Z-5 | Z-19 | ZBD-3 | ET12 | ET2 | blue | 3.0 |
| | | | | 84% | 15% | 1% | | | | |
| Example 26 | HT16 | HT3 | HT12 | Z-5 | Z-20 | ZBD-3 | ET12 | ET2 | blue | 3.0 |
| | | | | 84% | 15% | 1% | | | | |

### Effect of Freely Rotatable Single Bond in Organic Compound Contained in Light-emitting Layer Being Carbon-Carbon Bond

Comparisons of Example 1 with Comparative Examples 1 to 3 revealed that the present invention, in which the organic compounds in the light-emitting layer had only carbon-carbon bonds as freely rotatable single bonds, exhibited better durability characteristics than Comparative Examples 1 to 3, in which carbon-nitrogen bonds were contained. This is because the freely rotatable single bonds in the organic compounds contained in the light-emitting layer in Example 1 are only carbon-carbon bonds, and thus the bond energy is high, so that decomposition and reaction are less likely to occur due to charges or in an excited state during the operation of the light-emitting device.

### Effect of at Least One Carbon Atom of Freely Rotatable Carbon-Carbon Bond of Second Organic Compound Being Sp² Carbon Atom

Comparisons of Examples 2 to 4 with Comparative Example 4 reveal that Example 4, in which the second organic compound contains methyl groups, exhibits better durability characteristics than Comparative Example 4, in which tert-butyl groups are contained, and Example 3, in which only aryl groups are contained, exhibits even better durability characteristics. This is because in the examples, in which the bond energies of molecules of the organic compounds in the light-emitting layer are high, decomposition and reaction are less likely to occur due to charges or in an excited state during the operation of the light-emitting device.

### Effect of Stable Structure of First Organic Compound

A comparison of Example 1 with Example 5 reveals that Example 5 exhibits better durability characteristics than Example 1. This is because the first organic compound in Example 1 contains an alkyl group, whereas the first organic compound in Example 5 is composed only of aryl groups, and thus decomposition or reaction is less likely to occur due to charges or in an excited state during the operation of the light-emitting device.

### Effect of Stable Structure of Third Organic Compound

A comparison of Example 5 with Example 6 reveals that Example 6 exhibits better durability characteristics than Example 5. This is because the third organic compound in Example 5 contains alkyl groups, whereas the third organic compound in Example 6 is composed only of aryl groups, and thus decomposition or reaction is less likely to occur due to charges or in an excited state during the operation of the light-emitting device.

### Effect of Hole-Trapping Property of First Organic Compound

A Comparison between Examples 8 and 9 reveals that Example 8 exhibits better durability characteristics than Example 9. This is because the HOMO difference between the third organic compound and the first organic compound is 0.1 eV in Example 9, whereas it is 0.2 eV in Example 8, and thus Example 8 exhibits a stronger hole-trapping property and a better carrier balance.

### Effect of Electron-trapping Property of First Organic Compound

A Comparison between Examples 6 and 13 reveals that Example 13 exhibits better durability characteristics than Example 6. This is because the LUMO difference between the third organic compound and the first organic compound is 0.1 eV in Example 6, whereas it is 0.4 eV in Example 13, and thus Example 13 exhibits a stronger electron-trapping property and a better carrier balance.

### Example 27: Synthesis of Z-18

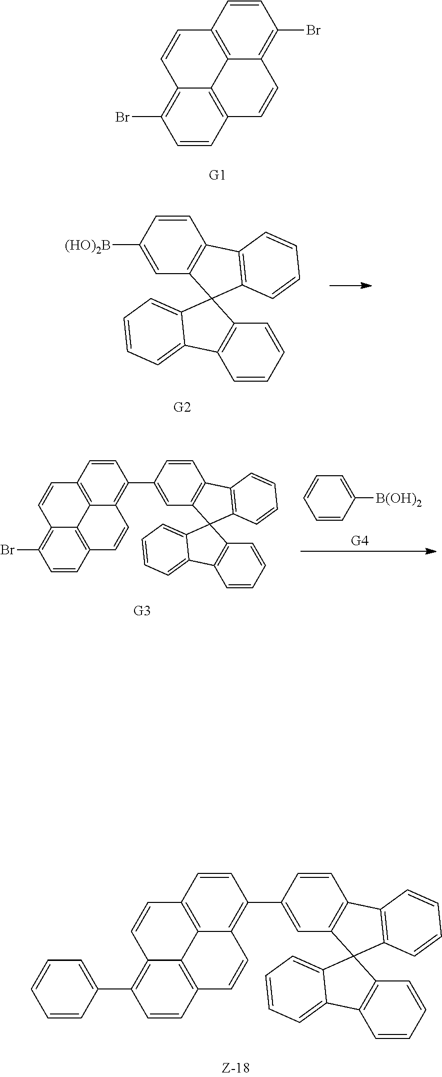

### Synthesis of Compound Z-18

The following reagents and solvents were placed in a 50-ml recovery flask.
Compound G1: 1.4 g (3.9 mmol)
Compound G2: 1.4 g (3.9 mmol)
Pd(PPh₃)₄: 0.23 g (0.2 mmol)
Potassium carbonate: 2.7 g
Toluene: 20 ml
Ethanol: 6 ml
H₂O: 8 ml

The reaction solution was heated to reflux with stirring for 6 hours under a stream of nitrogen. After the completion of the reaction, the reaction mixture was concentrated, followed by the addition of water. The mixture was stirred and then filtered. The resulting residue was collected. The residue was dissolved in chlorobenzene. The resulting solution was subjected to purification by column chromatography (chlorobenzene:heptane). Recrystallization was then performed from chlorobenzene/heptane to give 0.72 g (yield: 31%) of G3 as a white solid.

G3 was subjected to mass spectrometry with MALDI-TOF-MS (Autoflex LRF, manufactured by Bruker).
[MALDI-TOF-MS]
Measured value: m/z=596, calculated value: C₄₁H₂₃Br=596

The following reagents and solvents were placed in a 50-ml recovery flask.
Compound G3: 0.50 g (0.84 mmol)
Compound G4: 0.11 g (0.92 mmol)
Pd(PPh₃)₄: 49 mg (0.04 mmol)
Sodium carbonate: 0.6 g
Toluene: 10 ml
Ethanol: 4 ml
H₂O: 6 ml

The reaction solution was heated to reflux with stirring for 6 hours under a stream of nitrogen. After the completion of the reaction, the mixture was filtered. The resulting residue was collected. The residue was dissolved in chlorobenzene. The resulting solution was subjected to purification by column chromatography (chlorobenzene:cyclohexane). Recrystallization was then performed from toluene to give 0.25 g (yield: 51%) of Z-18 as a pale yellow solid.

Z-18 was subjected to mass spectrometry with MALDI-TOF-MS (Autoflex LRF, manufactured by Bruker).
[MALDI-TOF-MS]
Measured value: m/z=593, calculated value: C₄₇H₂₈=593

### Examples 28 to 35: Syntheses of Exemplified Compounds

Table 10 presents Examples 28 to 35. Exemplified compounds were synthesized in the same manner as in Example 27, except that the raw materials G2 and G4 in Example 27 were changed. The measured values (m/z) from the mass spectrometry results, which were measured in the same manner as in Example 27, are also presented.

TABLE 12

| | Exemplified | | | |
|---|---|---|---|---|
| Example | compound | Raw material G2 | Raw material G4 | m/z |
| 28 | Z-19 | 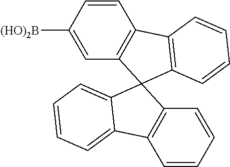 [SMILES: OB(O)c1ccc2c(c1)C1(c3ccccc3-c3ccccc31)c1ccccc1-2] | 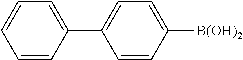 [SMILES: OB(O)c1ccc(-c2ccccc2)cc1] | 669 |
| 29 | Z-20 | 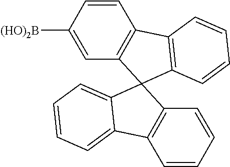 [SMILES: OB(O)c1ccc2c(c1)C1(c3ccccc3-c3ccccc31)c1ccccc1-2] | 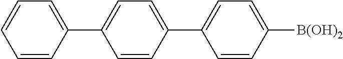 [SMILES: OB(O)c1ccc(-c2ccc(-c3ccccc3)cc2)cc1] | 745 |
| 30 | Z-21 | 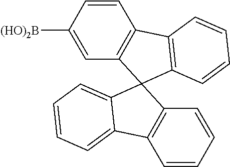 [SMILES: OB(O)c1ccc2c(c1)C1(c3ccccc3-c3ccccc31)c1ccccc1-2] | 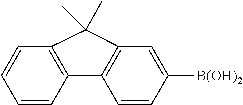 [SMILES: CC1(C)c2ccccc2-c2ccc(B(O)O)cc21] | 709 |
| 31 | Z-22 | 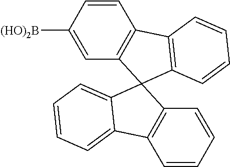 [SMILES: OB(O)c1ccc2c(c1)C1(c3ccccc3-c3ccccc31)c1ccccc1-2] | 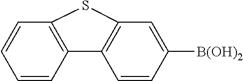 [SMILES: OB(O)c1ccc2c(c1)sc1ccccc12] | 699 |
| 32 | Z-23 | 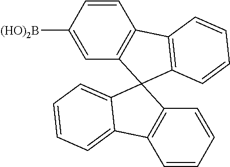 [SMILES: OB(O)c1ccc2c(c1)C1(c3ccccc3-c3ccccc31)c1ccccc1-2] | 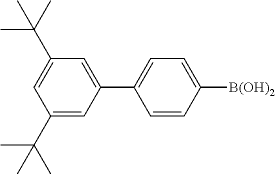 [SMILES: CC(C)(C)c1cc(-c2ccc(B(O)O)cc2)cc(C(C)(C)C)c1] | 781 |
| 33 | Z-24 | 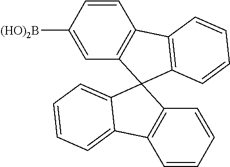 [SMILES: OB(O)c1ccc2c(c1)C1(c3ccccc3-c3ccccc31)c1ccccc1-2] | 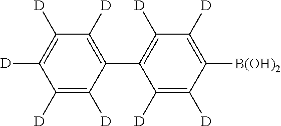 [SMILES: [2H]c1c([2H])c([2H])c(-c2c([2H])c([2H])c(B(O)O)c([2H])c2[2H])c([2H])c1[2H]] | 678 |
| 34 | Z-3 | 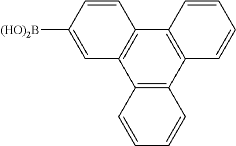 [SMILES: OB(O)c1ccc2c3ccccc3c3ccccc3c2c1] | 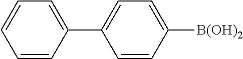 [SMILES: OB(O)c1ccc(-c2ccccc2)cc1] | 581 |
| 35 | Z-4 | 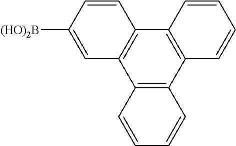 [SMILES: OB(O)c1ccc2c3ccccc3c3ccccc3c2c1] | 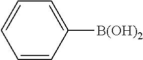 | 505 |

As described above, in Examples, three types of organic compounds, in which the freely rotatable single bonds consist of carbon-carbon bonds, were used as materials contained in each light-emitting layer, and the HOMO-LUMO relationships were adjusted. Thereby, charges were confined within the light-emitting layer, the concentration of recombination zones was inhibited, and the molecular structure of the compound constituting the light-emitting layer was stabilized, resulting in a highly durable organic light-emitting device.

According to the present invention, the organic light-emitting device having excellent driving durability characteristics can be provided, and excellent devices and apparatuses each including the organic light-emitting device can be provided.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

1. An organic light-emitting device, comprising:
a first electrode, a light-emitting layer, and a second electrode,
wherein the light-emitting layer contains a first organic compound, a second organic compound, and a third organic compound,
a freely rotatable single bond in each of the first organic compound, the second organic compound, and the third organic compound is a carbon-carbon bond,
at least one carbon atom of a freely rotatable carbon-carbon bond contained in the second organic compound is an sp² carbon atom, and
letting a HOMO energy level of the first organic compound be HOMO1, letting a LUMO energy level of the second organic compound be LUMO2, and letting HOMO and LUMO energy levels of the third organic compound be HOMO3 and LUMO3, [I] and [II] are satisfied:

|LUMO2|>|LUMO3| [I]

|HOMO3|>|HOMO1| [II].

2. The organic light-emitting device according to claim 1, wherein carbon atoms of the freely rotatable carbon-carbon bond in the second organic compound are each an sp² carbon atom.

3. The organic light-emitting device according to claim 1, wherein carbon atoms of freely rotatable carbon-carbon bonds contained in the first organic compound and the third organic compound are each an sp² carbon atom.

4. The organic light-emitting device according to claim 1, wherein amounts of the first to third organic compounds contained in the light-emitting layer satisfy the following relationship: the third organic compound>the first organic compound≥the second organic compound.

5. The organic light-emitting device according to claim 1, wherein the first organic compound, the second organic compound, and the third organic compound each contain no alkyl group.

6. The organic light-emitting device according to claim 1, wherein the second organic compound is a blue-light-emitting material.

7. The organic light-emitting device according to claim 1, wherein the second organic compound contains a fluoranthene skeleton.

8. The organic light-emitting device according to claim 1, wherein the third organic compound contains any one of an anthracene skeleton, a phenanthroline skeleton, a pyrene skeleton, a chrysene skeleton, a benzophenanthrene skeleton, a triphenylene skeleton, a fluoranthene skeleton, a benzochrysene skeleton, and a benzofluoran skeleton.

9. The organic light-emitting device according to claim 1, wherein the third organic compound contains a pyrene skeleton.

10. The organic light-emitting device according to claim 1, wherein the first organic compound is a compound represented by the following general formula [1]: 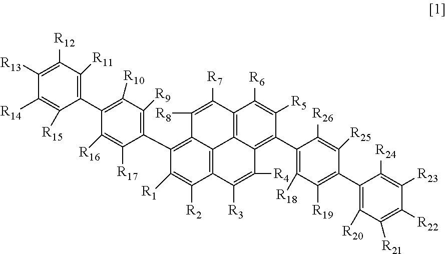
where in general formula [1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, R₁₀ and R₁₁, R₁₅ and R₁₆, R₁₉ and R₂₀, and R₂₄ and R₂₅ optionally form respective rings, each ring containing a carbon atom, an oxygen atom, a sulfur atom, a selenium atom, or a tellurium atom serving as a spacer, and R₁₁ to R₁₅ and R₂₀ to R₂₄ each optionally form a ring with an adjacent substituent.

11. The organic light-emitting device according to claim 1, wherein the first organic compound is a compound represented by the following general formula [2]: 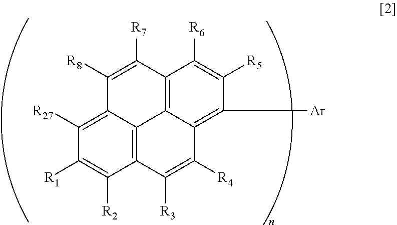
where in general formula [2], R₁ to R₈ and R₂₇ are each independently selected from a hydrogen atom, a deuterium atom, a substituted or unsubstituted alkyl group, a substituted or unsubstituted aryl group, and a substituted or unsubstituted heterocyclic group, and n is a natural number selected from 1 to 3.

12. The organic light-emitting device according to claim 11, wherein the first organic compound is a compound represented by any one of the following general formulae [1-1] to [1-3]: 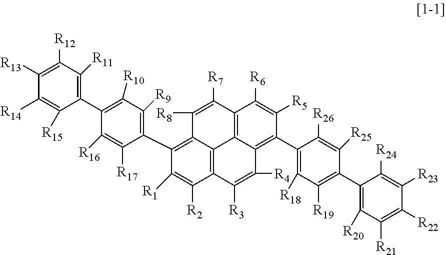
where in general formula [1-1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₀ and R₁₁, R₁₅ and R₁₆, R₁₉ and R₂₀, and R₂₄ and R₂₅ forms a ring with a carbon atom, an oxygen atom, a sulfur atom, a selenium atom, or a tellurium atom serving as a spacer, and R₁₁ to R₁₅ and R₂₀ to R₂₄ each optionally form a ring with an adjacent substituent, 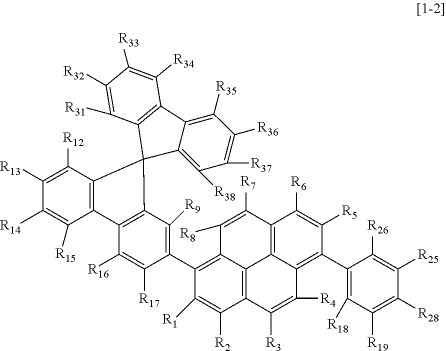
where in general formula [1-2], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, and R₁₈ and R₁₉, R₂₅ and R₂₆, and R₂₈ each optionally form a ring with an adjacent substituent, and 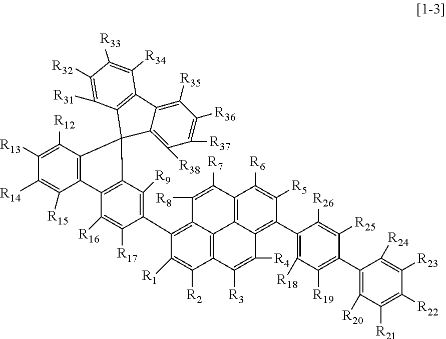
where in general formula [1-3], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₉ and R₂₀, and R₂₄ and R₂₅ forms a ring with a carbon atom, an oxygen atom, a sulfur atom, a selenium atom, or a tellurium atom serving as a spacer, and R₂₀ to R₂₄ each optionally form a ring with an adjacent substituent.

13. The organic light-emitting device according to claim 1, wherein the first organic compound is a compound with a skeleton represented by any one of the following general formulae [3-1] to [3-4]: 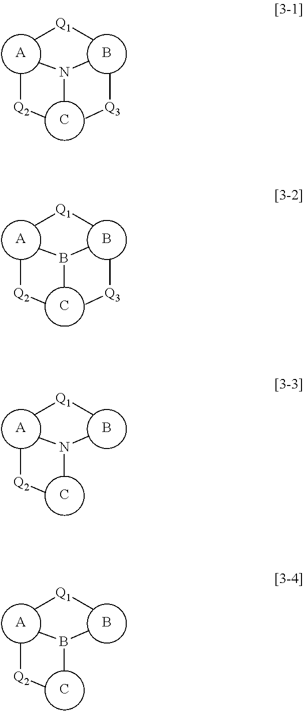
where in general formulae [3-1] to [3-4], cyclic units A to C are each independently selected from an aromatic hydrocarbon group and a heterocyclic group and are each optionally a fused ring, Q₁ to Q₃ are each independently selected from a direct bond, CR₁R₂, NR₃, an oxygen atom, a sulfur atom, a selenium atom, and a tellurium atom, substituents R₁ to R₃ are each independently selected from a substituted or unsubstituted alkyl group, a substituted or unsubstituted alkoxy group, a substituted or unsubstituted aryl group, and a substituted or unsubstituted heteroaryl group, and R₃ optionally forms a ring together with any one of adjacent cyclic units A to C.

14. The organic light-emitting device according to claim 1, further comprising a hole-blocking layer in contact with the light-emitting layer, wherein the hole-blocking layer contains a compound with a fused-ring skeleton composed of a hydrocarbon.

15. The organic light-emitting device according to claim 1, further comprising an electron-blocking layer in contact with the light-emitting layer, wherein a freely rotatable single bond in a compound contained in the electron-blocking layer is a carbon-carbon bond.

16. The organic light-emitting device according to claim 1, further comprising a second light-emitting layer disposed in contact with the first light-emitting layer, wherein the second light-emitting layer has an emission color different from an emission color of the first light-emitting layer.

17. A display apparatus, comprising multiple pixels, at least one of the multiple pixels including the organic light-emitting device according to claim 1 and a transistor coupled to the organic light-emitting device.

18. A photoelectric conversion apparatus, comprising an optical unit including multiple lenses, an image pickup device configured to receive light passing through the optical unit, and a display unit configured to display an image captured by the image pickup device,
wherein the display unit includes the organic light-emitting device according to claim 1.

19. An electronic apparatus, comprising a display unit including the organic light-emitting device according to claim 1, a housing provided with the display unit, and a communication unit disposed in the housing and configured to communicate with an outside.

20. A lighting apparatus, comprising a light source including the organic light-emitting device according to claim 1, and a light diffusion unit or an optical filter configured to transmit light emitted from the light source.

21. A moving object, comprising a lighting unit including the organic light-emitting device according to claim 1, and a body provided with the lighting unit.

22. An exposure light source for an electrophotographic image-forming apparatus, comprising the organic light-emitting device according to claim 1.

23. An organic compound represented by the following general formula [1-1]: 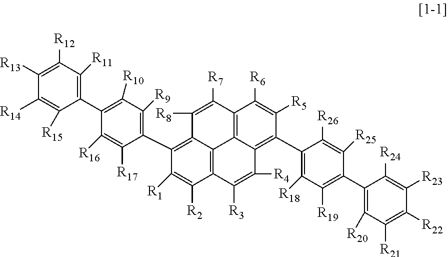
where in general formula [1-1], R₁ to R₂₆ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₀ and R₁₁, R₁₅ and R₁₆, R₁₉ and R₂₀, and R₂₄ and R₂₅ forms a ring with a carbon atom, an oxygen atom, a sulfur atom, a selenium atom, or a tellurium atom serving as a spacer, and R₁₁ to R₁₅ and R₂₀ to R₂₄ each optionally form a ring with an adjacent substituent.

24. An organic compound represented by the following general formula [1-2]: 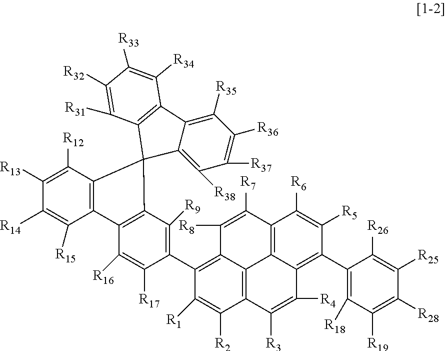
where in general formula [1-2], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, and R₁₈ and R₁₉, R₂₅ and R₂₆, and R₂₈ each optionally form a ring with an adjacent substituent.

25. The organic compound according to claim 23, wherein the organic compound is a compound represented by the following general formula [1-3]: 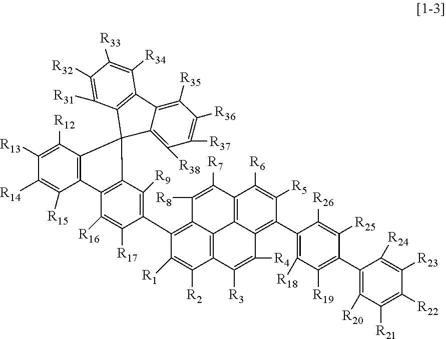
where in general formula [1-3], R₁ to R₃₈ are each independently selected from a hydrogen atom, a deuterium atom, a substituted or unsubstituted alkyl group, a substituted or unsubstituted alkenyl group, a substituted or unsubstituted aryl group, and a substituted or unsubstituted heterocyclic group, at least one pair of R₁₉ and R₂₀, and R₂₄ and R₂₅ forms a ring with a carbon atom, an oxygen atom, a sulfur atom, a selenium atom, or a tellurium atom serving as a spacer, and R₂₀ to R₂₄ each optionally form a ring with an adjacent substituent.
